(19) United States
(12) Patent Application Publication
(10) Pub. No.: US 2019/0008835 A1
(43) Pub. Date: Jan. 10, 2019

(54) COMPOSITIONS AND COMBINATIONS OF AUTOTAXIN INHIBITORS

(71) Applicants: X-Rx, Inc., Waltham, MA (US); Gilead Science, Inc., Foster City, CA (US)

(72) Inventor: John SUNDY, Foster City, CA (US)

(21) Appl. No.: 16/118,120

(22) Filed: Aug. 30, 2018

Related U.S. Application Data
(63) Continuation of application No. PCT/US2017/020678, filed on Mar. 3, 2017.
(60) Provisional application No. 62/303,547, filed on Mar. 4, 2016.

(51) Int. Cl. A61K 31/435; A61P 11/00; A61P 19/02

(52) U.S. Cl. CPC A61K 31/435; A61P 19/02; A61P 11/00

(57) ABSTRACT

Pharmaceutical compositions are provided having an autotaxin inhibitor compound and an additional therapeutic agent such as anti-fibrotics, anti-inflamatory agents, anti-cancer agents, and cardiovascular agents.

### CROSS-REFERENCES TO RELATED APPLICATIONS

This application claims priority to U.S. Provisional Application No. 62/303,547, filed Mar. 4, 2016, which is incorporated herein in its entirety for all purposes.

### BACKGROUND

Autotaxin (ATX) is a secreted enzyme of the ectonucleotide pyrophosphatase/phosphodiesterase family, and is also known as Ectonucleotide Pyrophosphatase/Phosphodiesterase 2 (ENPP-2 or NPP2). ATX plays a role in driving pathological conditions, including fibrosis, arthritic inflammation, neurodegeneration, neuropathic pain, and cancer. ATX is the fundamental regulator of the conversion of Lysophosphatidylcholine (LPC) to Lysophosphatidic Acid (LPA). LPA is a bioactive lipid that affects migration, proliferation, and survival of various cell types.

Inhibition of ATX has been shown to reduce LPA levels in pathological settings. Reduction of LPA may provide therapeutic benefits in diseases with unmet medical need, including cancer, lymphocyte homing, chronic inflammation, neuropathic pain, fibrotic diseases such as Idiopathic Pulmonary Fibrosis (IPF), thrombosis, and cholestatic pruritus which are caused, mediated and/or propagated by increased LPA levels and/or activation of ATX.

Fibrotic diseases are chronic, debilitating and often lethal pathologies driven by a dysregulated response to tissue or organ injury. Fibrosis can develop in the liver, kidney, lung, dermis, vasculature, gut and other sites. Fibrosis develops due to action of pathways including growth factors, cytokines, integrin and lipids.

ATX, LPA, and LPA Receptor (LPAR) pathways have been implicated in fibrotic disease. For example, profiling studies show increased levels of ATX, LPA and LPARs in various rodent models of fibrosis and in human patient fluids and biopsy tissue. LPA can induce proliferative, survival, and chemotactic responses in transformed cell lines, indicating that LPA may exert pro-inflammatory and profibrotic responses in cells known to be critical in fibrotic disease, including: fibroblasts, smooth muscle cells, macrophages, epithelial and endothelial cells, and leukocytes. Gene-targeted mouse models have implicated LPARs in fibrosis pathogenesis. Inhibitors of LPARs indicate that antagonism of receptors within this pathway blocked or reversed fibrosis in the lung, liver, kidney and skin in rodents. Cell type-specific gene targeting studies have showed that ATX plays a role in the development of lung fibrosis and inflammatory arthritis.

ATX and LPA have also been implicated in tumor progression and metastasis. ATX may be responsible for increased LPA levels in ascites and plasma of ovarian cancer patients since ATX converts LPC to LPA. Increased levels of LPA, altered receptor expression and altered responses to LPA may contribute to initiation, progression or outcome of ovarian cancer. LPA has also been linked to prostate, breast, melanoma, head and neck, bowel, brain and thyroid cancers.

LPA has been shown to promote tumor cell survival, proliferation, invasion and migration into neighboring tissues, which can result in the formation of metastases. Additionally, LPA promotes cytoskeletal remodeling that may enhance migratory and invasive properties of cells, which may contribute to cancer metastasis. These biological and pathobiological processes of LPA are initiated through the activation of G-protein coupled receptors.

Transcriptome analyses of more than 350 normal tissues and more than 1700 malignant tissues demonstrate that ATX is expressed in a variety of carcinomas and sarcomas, underscoring the potential contribution of LPA to metastatic disease.

Accordingly, when treating patients with diseases, such as cancer, lymphocyte homing, chronic inflammation, neuropathic pain, fibrotic diseases, thrombosis, and cholestatic pruritus it is desirable to lower LPA levels. This can be accomplished through inhibition of enzymes involved in LPA biosynthesis, such as ATX.

Since ATX is expressed in tumors and affects tumor cell proliferation and invasion into neighboring tissues both of which can lead to the formation of metastases, ATX is a target for anti-tumor therapy. Moreover, in angiogenesis, ATX, taken with other anti-angiogenetic factors, brings about blood vessel formation. Angiogenesis supplies tumors with nutrients during tumor growth. Therefore, inhibition of angiogenesis is a target for anti-tumor therapy, leading to starvation of a tumor.

ATX has also been implicated in nerve injury-induced neuropathic pain. LPA biosynthesis, through ATX, is the source of LPA for LPA1 receptor-mediated neuropathic pain. Therefore, targeted inhibition of ATX-mediated LPA biosynthesis may represent a novel treatment to prevent nerve injury-induced neuropathic pain.

Accordingly, there remains a need for ATX inhibitors having the potential to reach the clinic and obtain regulatory approval for use in the treatment and/or prophylaxis of physiological and/or pathophysiological conditions, such as cancer, lymphocyte homing, chronic inflammation, neuropathic pain, fibrotic diseases, thrombosis, and cholestatic pruritus which are caused, mediated and/or propagated by increased LPA levels and/or the activation of ATX.

### BRIEF SUMMARY

In one embodiment, the present invention provides a pharmaceutical composition including a therapeutically effective amount of an autotaxin inhibitor compound of Formula I:

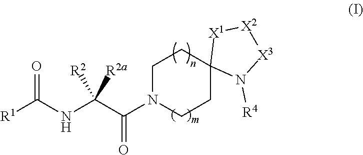

or a pharmaceutically acceptable salt thereof, an additional therapeutic agent, and a pharmaceutically acceptable carrier or excipient, wherein:
X¹ and X² are each independently selected from one or more of C₁₋₂ alkyl, C═O, NR³, or O;
X³ is independently selected from one or more of C₁₋₂ alkyl, C═O, NR³, O, or CR¹⁰R¹¹;
m and n are each independently selected from 0, 1 or 2;
R¹ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G¹ substituents;
R² is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G² substituents;
R^{2a} is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G^{2a} substituents;
R² and R^{2a} are each independently a linear structure, or, R² and R^{2a} are taken together with the carbon atom to which they are attached to form a 3-12 membered saturated or unsaturated ring, wherein said ring optionally includes one or more additional heteroatoms selected from O, N, or S(O)ₘ₁;
R³ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G³ substituents;
R⁴ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, heteroaryl-C₃₋₁₂heterocycloalkyl-, or pyridine-N-oxide, any of which is optionally substituted with one or more independent G⁴ substituents;
G¹, G², G^{2a}, G³, and G⁴ are each independently selected from one or more of H, D, halo, —CN, —CD₃, —OCD₃, -oxo-, —CF₃, —OCF₃, —OCHF₂, —NR⁵R⁶, —NO₂, —B(OH)₂, —PO(OR¹²)₂, —PO(OR¹²)R¹³, —CONR¹²OH, —C₀₋₁₂alkyl, —C₂₋₁₂alkenyl, —C₂₋₁₂alkynyl, C₃₋₁₂cycloalkyl-C₀₋₁₂alkyl-, C₃₋₁₂heterocycloalkyl-C₀₋₁₂alkyl-, aryl-C₀₋₁₂alkyl-, heteroaryl-C₀₋₁₂alkyl-, —OC₀₋₁₂alkyl, —S(O)ₙ₁R¹², —C(O)R¹², —C(O)NR¹²R¹³, —C(O)—C(O)NR¹²R¹³, —C(O)OR¹², —C(O)—C(O)OR¹², —OC(O)R¹², —NR¹²C(O)R¹³, —NR¹²C(O)OR¹³, —NR¹²S(O)₂R¹³, —(CR¹⁴R¹⁵)C(O)R¹³, —(CR¹⁴R¹⁵)C(O)OR¹², —(CR¹⁴R¹⁵)C(O)NR¹²R¹³, —(CR¹⁴R¹⁵)ₙ₁ S(O)₂NR¹²R¹³, —(CR¹⁴R¹⁵)ₙ₁NR¹²R¹³, —(CR¹⁴R¹⁵)ₙ₁OR¹², —(CR¹⁴R¹⁵)ₙ₁S(O)ₙ₂R¹², —NR¹⁶C(O)NR¹²R¹³, —NR¹⁶S(O)₂NR¹²R¹³ or —NR¹⁶S(O)NR¹²R¹³, any of which is optionally substituted with one or more independent Q¹ substituents;
Q¹ is selected from H, D, halo, —CN, —CD₃, —OCD₃, -oxo-, —CF₃, —OCF₃, —OCHF₂, —NO₂, —B(OH)₂, —PO(OR¹⁷)₂, —PO(OR¹⁷)R¹⁸, NR¹⁷R¹⁸, —CONR¹⁷OH, C₀₋₁₂alkyl-, —C₂₋₁₂ alkenyl, —C₂₋₁₂alkynyl, aryl-C₀₋₁₂alkyl-, heteroaryl-C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂cycloalkyl-, heteroaryl-C₃₋₁₂cycloalkyl-, C₃₋₁₂heterocycloalkyl-C₃₋₁₂cycloalkyl-, C₃₋₁₂cycloalkyl-C₃₋₁₂cycloalkyl-, C₁₋₁₂alkyl-C₃₋₁₂heterocycloalkyl-, C₃₋₁₂heterocycloalkyl-C₃₋₁₂heterocycloalkyl-, aryl-C₃₋₁₂heterocycloalkyl-, heteroaryl-C₃₋₁₂heterocycloalkyl-, —OC₀₋₁₂alkyl, —C(O)—C(O)NR¹⁷R¹⁸, —C(O)—C(O)OR¹⁷, —OC(O)R¹⁷, —NR¹⁷C(O)R¹⁸, —NR¹⁷S(O)₂R¹⁸, —(CR¹⁹R²⁰)ₙ₃C(O)R¹⁷, —(CR¹⁹R²⁰)ₙ₃C(O)OR¹⁷, —(CR¹⁹R²⁰)ₙ₃C(O)NR¹⁷R¹⁸, —(CR¹⁹R²⁰)ₙ₃S(O)₂NR¹⁷R¹⁸, —(CR¹⁹R²⁰)ₙ₃NR¹⁷R¹⁸, —(CR¹⁹R²⁰)ₙ₃OR¹⁷, —(CR¹⁹R²⁰)ₙ₃S(O)ₙ₄R¹⁷, —NR²¹C(O)NR¹⁷R¹⁸, —NR²¹S(O)₂NR¹⁷R¹⁸ or —NR²¹S(O)NR¹⁷R¹⁸, any of which is optionally substituted with one or more independent Q² substituents;
Q² is selected from one or more of H, D, halo, —CN, -oxo-, —CD₃, —OCD₃, —CF₃, —OCF₃, —OCHF₂, —NO₂, —B(OH)₂, —PO(OR²⁷)₂, —PO(OR²⁷)R²⁸—CONR²⁷OH, —CONR²⁷R²⁸C₀₋₁₂alkyl-, —C₂₋₁₂ alkenyl, —C₂₋₁₂ alkynyl, —OC₀₋₁₂alkyl, aryl-C₀₋₁₂alkyl-, heteroaryl-C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂cycloalkyl-, heteroaryl-C₃₋₁₂cycloalkyl-, C₃₋₁₂heterocycloalkyl-C₃₋₁₂cycloalkyl-, C₃₋₁₂cycloalkyl-C₃₋₁₂cycloalkyl-, C₁₋₁₂alkyl-C₃₋₁₂heterocycloalkyl-, C₃₋₁₂heterocycloalkyl-C₃₋₁₂heterocycloalkyl-, aryl-C₃₋₁₂heterocycloalkyl-, heteroaryl-C₃₋₁₂heterocycloalkyl-, C(O)—C(O)NR²⁷R²⁸, —C₀₋₁₂alkylC(O)OR²⁷, —C(O)—C(O)OR²⁷, —OC(O)R²⁷, —NR²⁷C(O)R²⁸, —NR²⁷C(O)OR²⁸—NR²⁷S(O)₂R²⁸, (CR²⁹R³⁰)ₙ₅C(O)R²⁷, —(CR²⁹R³⁰)ₙ₅C(O)OR²⁷, —(CR²⁹R³⁰)ₙ₅C(O)NR²⁷R²⁸, (CR²⁹R³⁰)ₙ₅S(O)₂NR²⁷R²⁸, —(CR²⁹R³⁰)ₙ₅NR²⁷R²⁸, —(CR²⁹R³⁰)ₙ₅OR²⁷, —(CR²⁹R³⁰)ₙ₅S(O)ₙ₆R²⁷, —NR³⁰C(O)NR²⁷R²⁸, —NR³⁰S(O)₂NR²⁷R²⁸ or —NR³⁰S(O)NR²⁷R²⁸ substituents, any of which may be optionally substituted;
R⁵, R⁶, R¹⁰, R¹¹, R¹², R¹³, R¹⁴, R¹⁵, and R¹⁶ are each independently selected from one or more of H, C₁₋₆alkyl-, C₃₋₈cycloalkyl-C₀₋₆alkyl-, C₃₋₈heterocycloalkyl-C₀₋₆alkyl-, aryl-C₀₋₆alkyl-, aryl-C₃₋₈cycloalkyl-, aryl-C₃₋₈heterocycloalkyl-, heteroaryl-C₁₋₆alkyl-, heteroaryl-C₃₋₈cycloalkyl- or heteroaryl-C₃₋₈heterocycloalkyl-, any of which may be optionally substituted;
R¹⁷, R¹⁸, R¹⁹, R²⁰, R²¹, R²⁷, R²⁸, R²⁹, and R³⁰ are each independently selected from H, C₁₋₆alkyl-, C₃₋₈cycloalkyl-C₀₋₆alkyl-, C₃₋₈heterocycloalkyl-C₀₋₆alkyl-, aryl-C₀₋₆alkyl-, aryl-C₃₋₈cycloalkyl-, aryl-C₃₋₈heterocycloalkyl-, heteroaryl-C₁₋₆alkyl-, heteroaryl-C₃₋₈cycloalkyl- or heteroaryl-C₃₋₈heterocycloalkyl-, any of which may be optionally substituted;
—NR⁵R⁶ and —NR¹²R¹³ are each independently a linear structure, or, R⁵ and R⁶, or R¹² and R¹³, respectively, are taken together with the nitrogen atom to which they are attached to form a 3-12 membered saturated or unsaturated ring, wherein said ring optionally includes one or more additional heteroatoms selected from O, N, or S(O)ₘ₂;
—CR¹⁰R¹¹ and —CR¹⁴R¹⁵ are each independently a linear structure, or, R¹⁰ and R¹¹, or R¹⁴ and R¹⁵ respectively, are taken together with the carbon atom to which they are attached to form a 3-12 membered saturated or unsaturated ring, wherein said ring optionally includes one or more heteroatoms selected from O, N, or S(O)ₘ₃;
—CR¹⁹R²⁰ is a linear structure, or, R¹⁹ and R²⁰ are taken together with the carbon atom to which they are attached to form a 3-12 membered saturated or unsaturated ring, wherein said ring optionally includes one or more additional heteroatoms selected from O, N, or S(O)ₘ₄;
—NR¹⁷R¹⁸ is a linear structure, or, R¹⁷ and R¹⁸ are taken together with the nitrogen atom to which they are attached to form a 3-12 membered saturated or unsaturated ring, wherein said ring optionally includes one or more additional heteroatoms selected from O, N, or S(O)ₘ₅;
—CR²⁹R³⁰ is a linear structure, or, R²⁹ and R³⁰ are taken together with the carbon atom to which they are attached to form a 3-12 membered saturated or unsaturated ring, wherein said ring optionally includes one or more additional heteroatoms selected from O, N, or S(O)ₘ₆; and
—NR²⁷R²⁸ is a linear structure, or, R²⁷ and R²⁸ are taken together with the nitrogen atom to which they are attached to form a 3-12 membered saturated or unsaturated ring, wherein said ring optionally includes one or more additional heteroatoms selected from O, N, or S(O)ₘ₇;
wherein m1, m2, m3, m4, m5, m6, m7, n1, n2, n3, n4, n5 and n6 are each independently selected from 0, 1 or 2.

In another embodiment, the present invention provides a pharmaceutical composition including a therapeutically effective amount of an autotaxin inhibitor compound of Formula I:

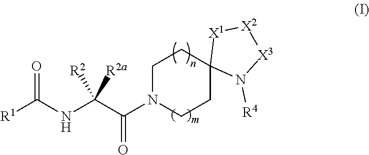

or a pharmaceutically acceptable salt thereof, an additional therapeutic agent, and a pharmaceutically acceptable carrier or excipient, wherein:
X¹ is selected from C₁₋₂ alkyl and C═O;
X² is selected from NR³ and O;
X³ is independently selected from one or more of C₁₋₂ alkyl or C═O;
m and n are each independently selected from 0, 1 or 2;
R¹ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G¹ substituents selected from H, halo, —CN, —CF₃, —OCF₃, —OCHF₂, —NR⁵R⁶, —NO₂, —C₀₋₁₂alkyl, —C₂₋₁₂alkenyl, —C₂₋₁₂alkynyl, C₃₋₁₂cycloalkyl-C₀₋₁₂alkyl-, C₃₋₁₂heterocycloalkyl-C₀₋₁₂alkyl-, aryl-C₀₋₁₂alkyl-, heteroaryl-C₀₋₁₂alkyl-, —OC₀₋₁₂alkyl, —S(O)ₙ₁R¹², —C(O)R¹², —C(O)NR¹²R¹³, —C(O)OR¹², —OC(O)R¹², —NR¹²C(O)R¹³, —NR¹²C(O)OR¹³, —NR¹²S(O)₂R¹³, or —(CR¹⁴R¹⁵)ₙ₁S(O)₂NR¹²R¹³;
R² is selected from C₀₋₁₂alkyl-, C₃₋₁₂cycloalkyl-C₀₋₁₂alkyl-,C₃₋₁₂heterocycloalkyl-C₀₋₁₂alkyl-, aryl-C₀₋₁₂alkyl-, or heteroaryl-C₀₋₁₂alkyl-, any of which is optionally substituted with one or more independent G² substituents selected from H, halo, —CN, —CF₃, —OCF₃, —OCHF₂, —NR⁵R⁶, —NO₂, —C₀₋₁₂alkyl, —C₂₋₁₂alkenyl, —C₂₋₁₂alkynyl, C₃₋₁₂cycloalkyl-C₀₋₁₂alkyl-, C₃₋₁₂heterocycloalkyl-C₀₋₁₂alkyl-, aryl-C₀₋₁₂alkyl-, heteroaryl-C₀₋₁₂alkyl-, —OC₀₋₁₂alkyl, —S(O)ₙ₁R¹², —C(O)R¹², —C(O)NR¹²R¹³, —C(O)OR¹², —OC(O)R¹², —NR¹²C(O)R¹³, —NR¹²C(O)OR¹³, NR¹²S(O)₂R¹³, or —(CR¹⁴R¹⁵)ₙ₁S(O)₂NR¹²R¹³;
R^{2a} is selected from C₀₋₁₂alkyl-, or C₃₋₁₂heterocycloalkyl-C₀₋₁₂alkyl-;
R² and R^{2a} are each independently a linear structure, or, R² and R^{2a} are taken together with the carbon atom to which they are attached to form a 3-12 membered saturated or unsaturated ring, wherein said ring optionally includes one or more additional heteroatoms selected from O, N, or S(O)ₘ₁;
R³ is selected from C₀₋₁₂alkyl-, or C₃₋₁₂cycloalkyl-C₀₋₁₂alkyl-, optionally substituted with one or more independent G³ substituents selected from H, halo, —CN, —CF₃, —OCF₃, —OCHF₂, —NR⁵R⁶, C₃₋₁₂cycloalkyl-C₀₋₁₂alkyl-, —S(O)ₙ₁R¹², —C(O)R¹², —C(O)NR¹²R¹³, —C(O)OR¹², —OC(O)R¹², —NR¹²C(O)R¹³, —NR¹²C(O)OR¹³, NR¹²S(O)₂R¹³, or —(CR¹⁴R¹⁵)ₙ₁S(O)₂NR¹²R¹³;
R⁴ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, heteroaryl-C₃₋₁₂heterocycloalkyl-, or pyridine-N-oxide, any of which is optionally substituted with one or more independent G⁴ substituents selected from H, D, halo, —CN, —CD₃, —OCD₃, -oxo-, —CF₃, —OCF₃, —OCHF₂, —NR⁵R⁶, —NO₂, —B(OH)₂, —PO(OR¹²)₂, —PO(OR¹²)R¹³, —CONR¹²OH, —C₀₋₁₂alkyl, —C₂₋₁₂alkenyl, —C₂₋₁₂alkynyl, C₃₋₁₂cycloalkyl-C₀₋₁₂alkyl-, C₃₋₁₂heterocycloalkyl-C₀₋₁₂alkyl-, aryl-C₀₋₁₂alkyl-, heteroaryl-C₀₋₁₂alkyl-, —OC₀₋₁₂alkyl, —S(O)ₙ₁R¹², C(O)R¹², —C(O)NR¹²R¹³, —C(O)OR¹², —OC(O)R¹², —NR¹²C(O)R¹³, —NR¹²C(O)OR¹³, —NR¹²S(O)₂R¹³, or —(CR¹⁴R¹⁵)ₙ₁S(O)₂NR¹²R¹³, wherein —OC₀₋₁₂alkyl is optionally substituted with one or more independent Q¹ substituents selected from halo, —CN, —CF₃, —OCF₃, —OCHF₂, NR¹⁷R¹⁸, —C₀₋₁₂alkyl, aryl-C₀₋₁₂alkyl-, heteroaryl-C₀₋₁₂alkyl-, C₃₋₁₂cycloalkyl-C₀₋₁₂alkyl-, C₃₋₁₂heterocycloalkyl-C₀₋₁₂alkyl-, —OC₀₋₁₂alkyl, —OC(O)R¹⁷, —NR¹⁷C(O)R¹⁸, —NR¹⁷S(O)₂R¹⁸, —(CR¹⁹R²⁰)ₙ₃C(O)R¹⁷, —(CR¹⁹R²⁰)ₙ₃C(O)OR¹⁷, —(R¹⁹R²⁰)ₙ₃C(O)NR¹⁷R¹⁸, —(CR¹⁹R²⁰)ₙ₃S(O)₂NR¹⁷R¹⁸, —(CR¹⁹R²⁰)ₙ₃NR¹⁷R¹⁸, or —(CR¹⁹R²⁰)ₙ₃OR¹⁷;
R⁵, R⁶, R¹², R¹³, R¹⁴ and R¹⁵ are each independently selected from one or more of H, C₁₋₆alkyl-, C₃₋₈cycloalkyl-C₀₋₆alkyl-,C₃₋₈heterocycloalkyl-C₀₋₆alkyl-, aryl-C₀₋₆alkyl-, or heteroaryl-C₀₋₆alkyl-;
R¹⁷, R¹⁸, R¹⁹, and R²⁰ are each independently selected from H, C₁₋₆alkyl-, C₃₋₈cycloalkyl-C₀₋₆alkyl-, C₃₋₈heterocycloalkyl-C₀₋₆alkyl-, aryl-C₀₋₆alkyl-, or heteroaryl-C₁₋₆alkyl-;
—NR⁵R⁶ and —NR¹²R¹³ are each independently a linear structure;
—CR¹⁴R¹⁵ is a linear structure;
—CR¹⁹R²⁰ is a linear structure; and
—NR¹⁷R¹⁸ is a linear structure;
wherein m1, n1 and n3 are each independently selected from 0, 1 or 2.

### DETAILED DESCRIPTION OF THE INVENTION

### I. General

The present invention includes pharmaceutical compositions of autotaxin inhibitors and at least one additional therapeutic agent, such as anti-inflammatory agents, anti-fibrotic agents, oncology agents, cardiovascular agents, and others.

### II. Definitions

Except where otherwise indicated, the following general conventions and definitions apply. Unless otherwise indicated herein, language and terms are to be given their broadest reasonable interpretation as understood by the skilled artisan. Any examples given are non-limiting.

Any section headings or subheadings herein are for the reader's convenience and/or formal compliance and are non-limiting.

A recitation of a compound herein is open to and embraces any material or composition containing the recited compound (e.g., a composition containing a racemic mixture, tautomers, epimers, stereoisomers, impure mixtures, etc.). In that a salt, solvate, or hydrate, polymorph, or other complex of a compound includes the compound itself, a recitation of a compound embraces materials containing such forms. Isotopically labeled compounds are also encompassed except where specifically excluded. For example, hydrogen is not limited to hydrogen containing zero neutrons. For example, deuterium is referred to herein as “D” and means a hydrogen atom having one neutron.

The term “active agent” of the present invention means a compound of the invention in any salt, polymorph, crystal, solvate, or hydrated form.

The term “pharmaceutically acceptable salt(s)” is known in the art and includes salts of acidic or basic groups which can be present in the compounds and prepared or resulting from pharmaceutically acceptable bases or acids.

The term “substituted” and substitutions contained in formulas herein refer to the replacement of one or more hydrogen radicals in a given structure with a specified radical, or, if not specified, to the replacement with any chemically feasible radical. When more than one position in a given structure can be substituted with more than one substituent selected from specified groups, the substituents can be either the same or different at every position (independently selected) unless otherwise indicated. In some cases, two positions in a given structure can be substituted with one shared substituent. It is understood that chemically impossible or highly unstable configurations are not desired or intended, as the skilled artisan would appreciate.

In descriptions and claims where subject matter (e.g., substitution at a given molecular position) is recited as being selected from a group of possibilities, the recitation is specifically intended to include any subset of the recited group. In the case of multiple variable positions or substituents, any combination of group or variable subsets is also contemplated. Unless indicated otherwise, a substituent, diradical or other group referred to herein can be bonded through any suitable position to a referenced subject molecule. For example, the term “indolyl” includes 1-indolyl, 2-indolyl, 3-indolyl, etc.

The convention for describing the carbon content of certain moieties is “(C_{a-b})” or “Cₐ-C_{b}” meaning that the moiety can contain any number of from “a” to “b” carbon atoms. C₀alkyl means a single covalent chemical bond when it is a connecting moiety and a hydrogen when it is a terminal moiety. Similarly, “x-y” can indicate a moiety containing from x to y atoms, e.g., 5-6 heterocycloalkyl means a heterocycloalkyl having either five or six ring members. “C_{x-y}” may be used to define number of carbons in a group. For example, “C₀₋₁₂alkyl” means alkyl having 0-12 carbons, wherein C₀alkyl means a single covalent chemical bond when a linking group and means hydrogen when a terminal group.

The term “absent,” as used herein to describe a structural variable (e.g., “—R— is absent”) means that diradical R has no atoms, and merely represents a bond between other adjoining atoms, unless otherwise indicated.

Unless otherwise indicated (such as by a connecting“—”), the connections of compound name moieties are at the rightmost recited moiety. That is, the substituent name starts with a terminal moiety, continues with any bridging moieties, and ends with the connecting moiety. For example, “heteroarylthioC₁₋₄alkyl is a heteroaryl group connected through a thio sulfur to a C₁₋₄ alkyl, which alkyl connects to the chemical species bearing the substituent.

The term “aliphatic” means any hydrocarbon moiety, and can contain linear, branched, and cyclic parts, and can be saturated or unsaturated.

The term “alkyl” means any saturated hydrocarbon group that is straight-chain or branched. Examples of alkyl groups include methyl, ethyl, propyl, 2-propyl, n-butyl, iso-butyl, tert-butyl, pentyl, and the like.

The term “alkenyl” means any ethylenically unsaturated straight-chain or branched hydrocarbon group. Representative examples include, but are not limited to, ethenyl, 1-propenyl, 2-propenyl, 1-, 2-, 3-butenyl, and the like.

The term “alkynyl” means any acetylenically unsaturated straight-chain or branched hydrocarbon group. Representative examples include, but are not limited to, ethynyl, 1-propynyl, 2-propynyl, 1-, 2-, or 3-butynyl, and the like.

The term “alkoxy” means —O-alkyl, —O-alkenyl, or —O-alkynyl. “Haloalkoxy” means an —O-(haloalkyl) group. Representative examples include, but are not limited to, trifluoromethoxy, tribromomethoxy, and the like.

“Haloalkyl” means an alkyl, preferably lower alkyl, that is substituted with one or more same or different halo atoms.

“Hydroxyalkyl” means an alkyl, preferably lower alkyl, that is substituted with one, two, or three hydroxy groups; e.g., hydroxymethyl, 1 or 2-hydroxyethyl, 1,2-, 1,3-, or 2,3-dihydroxypropyl, and the like.

The term “alkanoyl” means —C(O)-alkyl, —C(O)-alkenyl, or —C(O)-alkynyl.

“Alkylthio” means an —S-(alkyl) or an —S-(unsubstituted cycloalkyl) group.

Representative examples include, but are not limited to, methylthio, ethylthio, propylthio, butylthio, cyclopropylthio, cyclobutylthio, cyclopentylthio, cyclohexylthio, and the like.

The term “cyclic” means any ring system with or without heteroatoms (N, O, or S(O)₀₋₂), and which can be saturated, partially saturated, or unsaturated. Ring systems can be bridged and can include fused rings. The size of ring systems may be described using terminology such as “_{x-y}cyclic,” which means a cyclic ring system that can have from x to y ring atoms. For example, the term “₉₋₁₀carbocyclic” means a 5,6 or 6,6 fused bicyclic carbocyclic ring system which can be saturated, unsaturated or aromatic. It also means a phenyl fused to one 5 or 6 membered saturated or unsaturated carbocyclic group. Non limiting examples of such groups include naphthyl, 1,2,3,4 tetrahydronaphthyl, indenyl, indanyl, and the like.

The term “carbocyclic” means a cyclic ring moiety containing only carbon atoms in the ring(s) without regard to aromaticity. A 3-10 membered carbocyclic means chemically feasible monocyclic and fused bicyclic carbocyclics having from 3 to 10 ring atoms. Similarly, a 4-6 membered carbocyclic means monocyclic carbocyclic ring moieties having 4 to 6 ring carbons, and a 9-10 membered carbocyclic means fused bicyclic carbocyclic ring moieties having 9 to 10 ring carbons.

The term “cycloalkyl” means a non-aromatic 3-12 carbon mono-cyclic, bicyclic, or polycyclic aliphatic ring moiety. Cycloalkyl can be bicycloalkyl, polycycloalkyl, bridged, or spiroalkyl. One or more of the rings may contain one or more double bonds but none of the rings has a completely conjugated pielectron system. Examples, without limitation, of cycloalkyl groups are cyclopropane, cyclobutane, cyclopentane, cyclopentene, cyclohexane, cyclohexadiene, adamantane, cycloheptane, cycloheptatriene, and the like.

The term “unsaturated carbocyclic” means any cycloalkyl containing at least one double or triple bond. The term “cycloalkenyl” means a cycloalkyl having at least one double bond in the ring moiety.

The terms “bicycloalkyl” and “polycycloalkyl” mean a structure consisting of two or more cycloalkyl moieties that have two or more atoms in common. If the cycloalkyl moieties have exactly two atoms in common they are said to be “fused”. Examples include, but are not limited to, bicyclo[3.1.0]hexyl, perhydronaphthyl, and the like. If the cycloalkyl moieties have more than two atoms in common they are said to be “bridged”. Examples include, but are not limited to, bicyclo[2.2.1)heptyl (“norbornyl”), bicyclo[2.2.2)octyl, and the like.

The term “spiroalkyl” means a structure consisting of two cycloalkyl moieties that have exactly one atom in common. Examples include, but are not limited to, spiro[4.5]decyl, spiro[2.3]hexyl, and the like.

The term “aromatic” means a planar ring moieties containing 4n+2 pi electrons, wherein n is an integer.

The term “aryl” means aromatic moieties containing only carbon atoms in its ring system. Nonlimiting examples include phenyl, naphthyl, and anthracenyl. The terms “aryl-alkyl” or “arylalkyl” or 10 “aralkyl” refer to any alkyl that forms a bridging portion with a terminal aryl.

“Aralkyl” means alkyl that is substituted with an aryl group as defined above; e.g., —CH₂ phenyl, —(CH₂)₂phenyl, —(CH₂)₃phenyl, CH₃CH(CH₃)CH₂phenyl, and the like and derivatives thereof.

The term “heterocyclic” means a cyclic ring moiety containing at least one heteroatom (N, O, or S(O)₀₋₂), including heteroaryl, heterocycloalkyl, including unsaturated heterocyclic rings.

The term “heterocycloalkyl” means a non-aromatic monocyclic, bicyclic, or polycyclic heterocyclic ring moiety of 3 to 12 ring atoms containing at least one ring having one or more heteroatoms. The rings may also have one or more double bonds. However, the rings do not have a completely conjugated pielectron system. Examples, without limitation, of heterocycloalkyl rings include azetidine, oxetane, tetrahydrofuran, tetrahydropyran, oxepane, oxocane, thietane, thiazolidine, oxazolidine, oxazetidine, pyrazolidine, isoxazolidine, isothiazolidine, tetrahydrothiophene, tetrahydrothiopyran, thiepane, thiocane, azetidine, pyrrolidine, piperidine, N-methylpiperidine, azepane, 1,4-diazapane, azocane, [1,3]dioxane, oxazolidine, piperazine, homopiperazine, morpholine, thiomorpholine, 1,2,3,6-tetrahydropyridine and the like. Other examples of heterocycloalkyl rings include the oxidized forms of the sulfur-containing rings. Thus, tetrahydrothiophene-1-oxide, tetrahydrothiphene-1,1-dioxide, thiomorpholine-1-oxide, thiomorpholine-1,1-dioxide, tetrahydrothiopyran-1-oxide, tetrahydrothiopyran-1,1-dioxide, thiazolidine-1-oxide, and thiazolidine-1,1-dioxide are also considered to be heterocycloalkyl rings. The term “heterocycloalkyl” also includes fused ring systems and can include a carbocyclic ring that is partially or fully unsaturated, such as a benzene ring, to form benzofused heterocycloalkyl rings. For example, 3,4-dihydro-1,4-benzodioxine, tetrahydroquinoline, tetrahydroisoquinoline and the like. The term “heterocycloalkyl” also includes heterobicycloalkyl, heteropolycycloalkyl, or heterospiroalkyl, which are bicycloalkyl, polycycloalkyl, or spiroalkyl, in which one or more carbon atom(s) are replaced by one or more heteroatoms selected from O, N, and S. For example, 2-oxa-spiro[3.3]heptane, 2,7-diazaspiro[4.5]decane, 6-oxa-2-thia-spiro[3.4]octane, octahydropyrrolo[1,2-a]pyrazine, 7-azabicyclo[2.2.1]heptane, 2-oxa-bicyclo[2.2.2]octane, and the like, are such heterocycloalkyls.

Examples of saturated heterocyclic groups include, but are not limited to oxiranyl, thiaranyl, aziridinyl, oxetanyl, thiatanyl, azetidinyl, tetrahydrofuranyl, tetrahydrothiophenyl, pyrrolidinyl, tetrahydropyranyl, tetrahydrothiopyranyl, piperidinyl, 1,4-dioxanyl, 1,4-oxathianyl, morpholinyl, 1,4-dithianyl, piperazinyl, 1,4-azathianyl, oxepanyl, thiepanyl, azepanyl, 1,4-dioxepanyl, 1,4-oxathiepanyl, 1,4-oxaazepanyl, 1,4-dithiepanyl, 1,4-thieazepanyl, 1,4-diazepanyl.

Non-aryl heterocyclic groups include saturated and unsaturated systems and can include groups having only 4 atoms in their ring system. The heterocyclic groups include benzofused ring systems and ring systems substituted with one or more oxo moieties. Recitation of ring sulfur is understood to include the sulfide, sulfoxide or sulfone where feasible. The heterocyclic groups also include partially unsaturated or fully saturated 4-10 membered ring systems, e.g., single rings of 4 to 8 atoms in size and bicyclic ring systems, including aromatic 6-membered aryl or heteroaryl rings fused to a non-aromatic ring. Also included are 4-6 membered ring systems (“4-6 membered heterocyclic”), which include 5-6 membered heteroaryls, and include groups such as azetidinyl and piperidinyl. Heterocyclics can be heteroatom attached where such is possible. For instance, a group derived from pyrrole can be pyrrol-1-yl (N-attached) or pyrrol-3-yl (C-attached). Other heterocyclics include imidazo(4,5-b)pyridin-3-yl and benzoimidazol-1-yl.

Examples of heterocyclic groups include pyrrolidinyl, tetrahydrofuranyl, tetrahydrothienyl, tetrahydropyranyl, tetrahydrothiopyranyl, piperidine, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl. 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, and the like.

The term “unsaturated heterocyclic” means a heterocycloalkyl containing at least one unsaturated bond. The term “heterobicycloalkyl” means a bicycloalkyl structure in which at least one carbon atom is replaced with a heteroatom. The term “heterospiroalkyl” means a spiroalkyl structure in which at least one carbon atom is replaced with a heteroatom.

Examples of partially unsaturated heteroalicyclic groups include, but are not limited to: 3,4-dihydro-2H-pyranyl, 5,6-dihydro-2H-pyranyl 2H-pyranyl, 1,2,3,4-tetrahydropyridinyl, and 1,2,5,6-tetrahydropyrdinyl.

The terms “heteroaryl” or “hetaryl” mean a monocyclic, bicyclic, or polycyclic aromatic heterocyclic ring moiety containing 5-12 atoms. Examples of such heteroaryl rings include, but are not limited to, furyl, thienyl, pyrrolyl, pyrazolyl, imidazolyl, oxazolyl, isoxazolyl, thiazolyl, isothiazolyl, triazolyl, oxadiazolyl, thiadiazolyl, tetrazolyl, pyridyl, pyridazinyl, pyrimidinyl, pyrazinyl, and triazinyl. The terms “heteroaryl” also include heteroaryl rings with fused carbocyclic ring systems that are partially or fully unsaturated, such as a benzene ring, to form a benzofused heteroaryl. For example, benzimidazole, benzoxazole, benzothiazole, benzofuran, quinoline, isoquinoline, quinoxaline, indazole, imidazo[1,2-a]pyridine, 3-methyl-2-oxo-2,3-dihydrobenzo[d]oxazol-5-yl, 2-methyl-2H-indazol-5-yl, 3-methylimidazo[1,5-a]pyridine, 2-methyl-1H-benzo[d]imidazole, 1H-pyrrolo[2,3-b]pyridine, 3,4-Dihydro-2H-benzo[b][1,4]oxazine, 2-oxo-2,3-dihydrobenzo[d]oxazole, 3-oxo-3,4-dihydro-2H-benzo[b][1,4]oxazine, 2,3-Dihydrobenzo[b][1,4]dioxine, 2-methyl-[1,2,4]triazolo[1,5-a]pyridine, and the like. Furthermore, the terms “heteroaryl” include fused 5-6, 5-5, 6-6 ring systems, optionally possessing one nitrogen atom at a ring junction. Examples of such hetaryl rings include, but are not limited to, pyrrolopyrimidinyl, imidazo[1,2-a]pyridinyl, imidazo[2,1-b]thiazolyl, imidazo[4,5-b]pyridine, pyrrolo[2,1-f][1,2,4]triazinyl, and the like. Heteroaryl groups may be attached to other groups through their carbon atoms or the heteroatom(s), if applicable. For example, pyrrole may be connected at the nitrogen atom or at any of the carbon atoms.

Heteroaryls include, e.g., 5 and 6 membered monocyclics such as pyrazinyl and pyridinyl, and 9 and 10 membered fused bicyclic ring moieties, such as quinolinyl. Other examples of heteroaryl include quinolin-4-yl, 7-methoxy-quinolin-4-yl, pyridin-4-yl, pyridin-3-yl, and pyridin-2-yl. Other examples of heteroaryl include pyridinyl, imidazolyl, pyrimidinyl, pyrazolyl triazolyl, pyrazinyl, tetrazolyl, furanyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, furopyridinyl, and the like. Examples of 5-6 membered heteroaryls include, thiophenyl, isoxazolyl, 1,2,3-triazolyl, 1,2,3-oxadiazolyl, 1,2,3-thiadiazolyl, 1,2,4-triazolyl, 1,3,4-oxadiazolyl, 1,3,4-thiadiazolyl, 1,2,5-oxadiazolyl, 1,2,5-thiadiazolyl, pyridyl, pyridazinyl, pyrimidinyl, pyrazinyl, 1,2,4 oxadiazolyl, 1,2,5-triazinyl, 1,3,5-triazinyl, 6-oxo-1,6-dihydropyridine, and the like.

“Heteroaralkyl” group means alkyl, preferably lower alkyl, that is substituted with a heteroaryl group; e.g., —CH₂ pyridinyl, —(CH₂)₂pyrimidinyl, —(CH₂)₃imidazolyl, and the like, and derivatives thereof.

A pharmaceutically acceptable heteroaryl is one that is sufficiently stable to be attached to a compound of the invention, formulated into a pharmaceutical composition and subsequently administered to a patient in need thereof.

Examples of monocyclic heteroaryl groups include, but are not limited to: pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl tetrazolyl, pyridinyl, pyridazinyl, pyrimidinyl, pyrazinyl.

Examples of fused ring heteroaryl groups include, but are not limited to: benzoduranyl, benzothiophenyl, indolyl, benzimidazolyl, indazolyl, benzotriazolyl, pyrrolo[2,3-b]pyridinyl, pyrrolo[2,3-c]pyridinyl, pyrrolo[3,2-c]pyridinyl, pyrrolo[3,2-b]pyridinyl, imidazo[4,5-b]pyridinyl, imidazo[4,5-c]pyridinyl, pyrazolo[4,3-d]pyridinyl, pyrazolo[4,3-c]pyridinyl, pyrazolo]3,4-c]pyridinyl, pyrazolo[3,4-b]pyridinyl, isoindolyl, indazolyl, purinyl, indolinyl, imidazo[1,2-a]pyridinyl, imidazo[1,5-a]pyridinyl, pyrazolo[1,5-a]pyridinyl, pyrrolo[1,2-b]pyridazinyl, imidazo[1,2-c]pyrimidinyl, quinolinyl, isoquinolinyl, cinnolinyl, azaquinazoline, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-b]pyrazinyl, pyrido[3,4-b]pyrazinyl, pyrimido[5,4-d]pyrimidinyl, pyrimido[2,3-b]pyrazinyl, pyrimido[4,5-d]pyrimidinyl.

“Arylthio” means an —S-aryl or an —S-heteroaryl group, as defined herein. Representative examples include, but are not limited to, phenylthio, pyridinylthio, furanylthio, thienylthio, pyrimidinylthio, and the like and derivatives thereof.

The term “9-10 membered heterocyclic” means a fused 5,6 or 6,6 bicyclic heterocyclic ring moiety, which can be saturated, unsaturated or aromatic. The term “9-10 membered fused bicyclic heterocyclic” also means a phenyl fused to one 5 or 6 membered heterocyclic group. Examples include benzofuranyl, benzothiophenyl, indolyl, benzoxazolyl, 3H-imidazo[4,5-c]pyridin-yl, dihydrophthazinyl, 1H-3,5-imidazo[4,5-c]pyridin-1-yl, imidazo[4,5-b]pyridyl, 1,3 benzo[1,3]dioxolyl, 2H-chromanyl, isochromanyl, 5-oxo-2,3-dihydro-5H-[1,3]thiazolo[3,2-a]pyrimidyl, 1,3-benzothiazolyl, 1,4,5,6 tetrahydropyridazyl, 1,2,3,4,7,8-hexahydropteridinyl, 2-thioxo-2,3,6,9-tetrahydro-1H-purin-8-yl, 3,7-dihydro-1H-purin-8-yl, 3,4-dihydropyrimidin-1-yl, 2,3-dihydro-1,4-benzodioxinyl, benzo[1,3]dioxolyl, 2H-chromenyl, chromanyl, 3,4-dihydrophthalazinyl, 2,3-ihydro-1H-indolyl, 1,3-dihydro-2H-isoindol-2-yl, 2,4,7-trioxo-1,2,3,4,7,8-hexahydropteridin-yl, thieno[3,2-d]pyrimidinyl, 4-oxo-4,7-dihydro-3H-pyrrolo[2,3-d]pyrimidin-yl, 1,3-dimethyl-6-oxo-2-thioxo-2,3,6,9-tetrahydro-1H-purinyl, 1,2-dihydroisoquinolinyl, 2-oxo-1,3-benzoxazolyl, 2,3-dihydro-5H-1,3-thiazolo-[3,2-a]pyrimidinyl, 5,6,7,8-tetrahydro-quinazolinyl, 4-oxochromanyl, 1,3-benzothiazolyl, benzimidazolyl, benzotriazolyl, purinyl, furylpyridyl, thiophenylpyrimidyl, thiophenylpyridyl, pyrrolylpiridyl, oxazolylpyridyl, thiazolylpiridyl, 3,4-dihydropyrimidin-1-yl imidazolylpyridyl, quinoliyl, isoquinolinyl, quinazolinyl, quinoxalinyl, naphthyridinyl, pyrazolyl[3,4]pyridine, 1,2-dihydroisoquinolinyl, cinnolinyl, 2,3-dihydro-benzo[1,4]dioxin4-yl, 4,5,6,7-tetrahydro-benzo[b]-thiophenyl-2-yl, 1,8-naphthyridinyl, 1,5-napthyridinyl, 1,6-naphthyridinyl, 1,7-napthyridinyl, 3,4-dihydro-2H-1,4-benzothiazine, 4,8-dihydroxy-quinolinyl, 1-oxo-1,2-dihydroisoquinolinyl, 4-phenyl-[1,2,3]thiadiazolyl, and the like.

The term “aryloxy” means an —O-aryl or an —O-heteroaryl group, as defined herein. Representative examples include, but are not limited to, phenoxy, pyridinyloxy, furanyloxy, thienyloxy, pyrimidinyloxy, pyrazinyloxy, and the like, and derivatives thereof.

The term “oxo” means a compound containing a carbonyl group. One in the art understands that an “oxo” requires a second bond from the atom to which the oxo is attached.

The term “halo” or “halogen” means fluoro, chloro, bromo, or iodo.

“Acyl” means a —C(O)R group, where R can be selected from the nonlimiting group of hydrogen or optionally substituted lower alkyl, trihalomethyl, unsubstituted cycloalkyl, aryl, or other suitable substituent.

“Thioacyl” or “thiocarbonyl” means a-C(S)R″ group, with R as defined above.

The term “protecting group” means a suitable chemical group that can be attached to a functional group and removed at a later stage to reveal the intact functional group. Examples of suitable protecting groups for various functional groups are described in. T.˜. Greene and P. G. M. Wuts, Protective Groups in Organic Synthesis, 2d Ed., John Wiley and Sons (1991 and later editions); L Fieser and M. Fieser, Fieser and Fieser's Reagents for Organic Synthesis, John Wiley and Sons (1994); and L Paquette, ed. Encyclopedia of Reagents for Organic Synthesis, John Wiley and Sons (1995). The term “hydroxy protecting group”, as used herein, unless otherwise indicated, includes Ac, CBZ, and various hydroxy protecting groups familiar to those skilled in the art, including the groups referred to in Greene.

The term “linear structure” mean a moiety having substituents that do not cyclize to form a ring system. A representative example includes, but is not limited to, a compound including —NR⁵R⁶ where any atoms of “R⁵” and any atoms of “R⁶” do not connect to form a ring.

As used herein, the term “pharmaceutically acceptable salt” means those salts which retain the biological effectiveness and properties of the parent compound and do not present insurmountable safety or toxicity issues.

The term “pharmaceutical composition” means an active compound in any form suitable for effective administration to a subject, e.g., a mixture of the compound and at least one pharmaceutically acceptable carrier.

As used herein, a “physiologically/pharmaceutically acceptable carrier” means a carrier or diluent that does not cause significant irritation to an organism and does not abrogate the biological activity and properties of the administered compound.

A “pharmaceutically acceptable excipient” means an inert substance added to a pharmaceutical composition to further facilitate administration of a compound. Examples, without limitation, of excipients include calcium carbonate, calcium phosphate, various sugars and types of starch, cellulose derivatives, gelatin. vegetable oils and polyethylene glycols.

The term “substituted” and substitutions contained in formulas herein refer to the replacement of one or more hydrogen radicals in a given structure with a specified radical, or, if not specified, to the replacement with any chemically feasible radical. When more than one position in a given structure can be substituted with more than one substituent selected from specified groups, the substituents can be either the same or different at every position (independently selected) unless otherwise indicated. In some cases, two positions in a given structure can be substituted with one shared substituent. It is understood that chemically impossible or highly unstable configurations are not desired or intended, as the skilled artisan would appreciate.

The terms “treat,” “treatment,” and “treating” means reversing, alleviating, or inhibiting the progress of the disorder or condition to which such term applies, or one or more symptoms of such disorder or condition. “Preventing” means partially or completely treating before the disorder or condition occurs.

“Therapeutically effective amount” means that amount of the compound being administered which will relieve to some extent one or more of the symptoms of the disorder being treated, or result in inhibition of the progress or at least partial reversal of the condition.

### III. Compounds

The compounds of Formula I may be prepared according to PCT Publication No. WO 2015/154023, incorporated herein in its entirety. In some embodiments, the compounds useful in the pharmaceutical compositions of the present invention include compounds of Formula I:

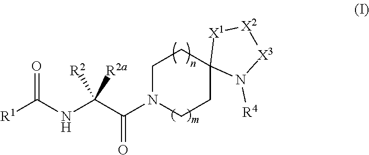

or a pharmaceutically acceptable salt thereof,

wherein
X¹ and X² are each independently selected from one or more of C₁₋₂ alkyl, C═O, NR³, or O;
X³ is independently selected from one or more of C₁₋₂ alkyl, C═O, NR³, O, or CR¹⁰R¹¹;
m and n are each independently selected from 0, 1 or 2;
R¹ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G¹ substituents;
R² is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G² substituents;
R^{2a} is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G^{2a} substituents;
R² and R^{2a} are each independently a linear structure, or, R² and R^{2a} are taken together with the carbon atom to which they are attached to form a 3-12 membered saturated or unsaturated ring, wherein said ring optionally includes one or more additional heteroatoms selected from O, N, or S(O)ₘ₁;
R³ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G³ substituents;
R⁴ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, heteroaryl-C₃₋₁₂heterocycloalkyl-, or pyridine-N-oxide, any of which is optionally substituted with one or more independent G⁴ substituents;
G¹, G², G^{2a}, G³, and G⁴ are each independently selected from one or more of H, D, halo, —CN, —CD₃, —OCD₃, -oxo-, —CF₃, —OCF₃, —OCHF₂, —NR⁵R⁶, —NO₂, —B(OH)₂, —PO(OR¹²)₂, —PO(OR¹²)R¹³, —CONR¹²OH, —C₀₋₁₂alkyl, —C₂₋₁₂alkenyl, —C₂₋₁₂alkynyl, C₃₋₁₂cycloalkyl-C₀₋₁₂alkyl-, C₃₋₁₂heterocycloalkyl-C₀₋₁₂alkyl-, aryl-C₀₋₁₂alkyl-, heteroaryl-C₀₋₁₂alkyl-, —OC₀₋₁₂alkyl, —S(O)ₙ₁R¹², —C(O)R¹², —C(O)NR¹²R¹³, —C(O)—C(O)NR¹²R¹³, —C(O)OR¹², —C(O)—C(O)OR¹², —OC(O)R¹², —NR¹²C(O)R¹³, —NR¹²C(O)OR¹³, —NR¹²S(O)₂R¹³, —(CR¹⁴R¹⁵)C(O)R¹³, —(CR¹⁴R¹⁵)C(O)OR¹², —(CR¹⁴R¹⁵)C(O)NR¹²R¹³, —(CR¹⁴R¹⁵)ₙ₁ S(O)₂NR¹²R¹³, —(CR¹⁴R¹⁵)ₙ₁NR¹²R¹³, —(CR¹⁴R¹⁵)ₙ₁OR¹², —(CR¹⁴R¹⁵)ₙ₁S(O)ₙ₂R¹², —NR¹⁶C(O)NR¹²R¹³, —NR¹⁶S(O)₂NR¹²R¹³ or —NR¹⁶S(O)NR¹²R¹³, any of which is optionally substituted with one or more independent Q¹ substituents;
Q¹ is selected from H, D, halo, —CN, —CD₃, —OCD₃, -oxo-, —CF₃, —OCF₃, —OCHF₂, —NO₂, —B(OH)₂, —PO(OR¹⁷)₂, —PO(OR¹⁷)R¹⁸, NR¹⁷R¹⁸, —CONR¹⁷OH, C₀₋₁₂alkyl-, —C₂₋₁₂ alkenyl, —C₂₋₁₂alkynyl, aryl-C₀₋₁₂alkyl-, heteroaryl-C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂cycloalkyl-, heteroaryl-C₃₋₁₂cycloalkyl-, C₃₋₁₂heterocycloalkyl-C₃₋₁₂cycloalkyl-, C₃₋₁₂cycloalkyl-C₃₋₁₂cycloalkyl-, C₁₋₁₂alkyl-C₃₋₁₂heterocycloalkyl-, C₃₋₁₂heterocycloalkyl-C₃₋₁₂heterocycloalkyl-, aryl-C₃₋₁₂heterocycloalkyl-, heteroaryl-C₃₋₁₂heterocycloalkyl-, —OC₀₋₁₂alkyl, —C(O)—C(O)NR¹⁷R¹⁸, —C(O)—C(O)OR¹⁷, —OC(O)R¹⁷, —NR¹⁷C(O)R¹⁸, —NR¹⁷S(O)₂R¹⁸, —(CR¹⁹R²⁰)ₙ₃C(O)R¹⁷, —(CR¹⁹R²⁰)ₙ₃C(O)OR¹⁷, —(CR¹⁹R²⁰)ₙ₃C(O)NR¹⁷R¹⁸, —(CR¹⁹R²⁰)ₙ₃S(O)₂NR¹⁷R¹⁸, —(CR¹⁹R²⁰)ₙ₃NR¹⁷R¹⁸, —(CR¹⁹R²⁰)ₙ₃OR¹⁷, —(CR¹⁹R²⁰)ₙ₃S(O)ₙ₄R¹⁷, —NR²¹C(O)NR¹⁷R¹⁸, —NR²¹S(O)₂NR¹⁷R¹⁸ or —NR²¹S(O)NR¹⁷R¹⁸, any of which is optionally substituted with one or more independent Q² substituents;
Q² is selected from one or more of H, D, halo, —CN, -oxo-, —CD₃, —OCD₃, —CF₃, —OCF₃, —OCHF₂, —NO₂, —B(OH)₂, —PO(OR²⁷)₂, —PO(OR²⁷)R²⁸—CONR²⁷OH, —CONR²⁷R²⁸C₀₋₁₂alkyl-, —C₂₋₁₂ alkenyl, —C₂₋₁₂ alkynyl, —OC₀₋₁₂alkyl, aryl-C₀₋₁₂alkyl-, heteroaryl-C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂cycloalkyl-, heteroaryl-C₃₋₁₂cycloalkyl-, C₃₋₁₂heterocycloalkyl-C₃₋₁₂cycloalkyl-, C₃₋₁₂cycloalkyl-C₃₋₁₂cycloalkyl-, C₁₋₁₂alkyl-C₃₋₁₂heterocycloalkyl-, C₃₋₁₂heterocycloalkyl-C₃₋₁₂heterocycloalkyl-, aryl-C₃₋₁₂heterocycloalkyl-, heteroaryl-C₃₋₁₂heterocycloalkyl-, C(O)—C(O)NR²⁷R²⁸, —C₀₋₁₂alkylC(O)OR²⁷, —C(O)—C(O)OR²⁷, —OC(O)R²⁷, —NR²⁷C(O)R²⁸, —NR²⁷C(O)OR²⁸—NR²⁷S(O)₂R²⁸, (CR²⁹R³⁰)ₙ₅C(O)R²⁷, —(CR²⁹R³⁰)ₙ₅C(O)OR²⁷, —(CR²⁹R³⁰)ₙ₅C(O)NR²⁷R²⁸, (CR²⁹R³⁰)ₙ₅S(O)₂NR²⁷R²⁸, —(CR²⁹R³⁰)ₙ₅NR²⁷R²⁸, —(CR²⁹R³⁰)ₙ₅OR²⁷, —(CR²⁹R³⁰)ₙ₅S(O)ₙ₆R²⁷, —NR³⁰C(O)NR²⁷R²⁸, —NR³⁰S(O)₂NR²⁷R²⁸ or —NR³⁰S(O)NR²⁷R²⁸ substituents, any of which may be optionally substituted;
R⁵, R⁶, R¹⁰, R¹¹, R¹², R¹³, R¹⁴, R¹⁵, and R¹⁶ are each independently selected from one or more of H, C₁₋₆alkyl-, C₃₋₈cycloalkyl-C₀₋₆alkyl-, C₃₋₈heterocycloalkyl-C₀₋₆alkyl-, aryl-C₀₋₆alkyl-, aryl-C₃₋₈cycloalkyl-, aryl-C₃₋₈heterocycloalkyl-, heteroaryl-C₁₋₆alkyl-, heteroaryl-C₃₋₈cycloalkyl- or heteroaryl-C₃₋₈heterocycloalkyl-, any of which may be optionally substituted;
R¹⁷, R¹⁸, R¹⁹, R²⁰, R²¹, R²⁷, R²⁸, R²⁹, and R³⁰ are each independently selected from H, C₁₋₆alkyl-, C₃₋₈cycloalkyl-C₀₋₆alkyl-, C₃₋₈heterocycloalkyl-C₀₋₆alkyl-, aryl-C₀₋₆alkyl-, aryl-C₃₋₈cycloalkyl-, aryl-C₃₋₈heterocycloalkyl-, heteroaryl-C₁₋₆alkyl-, heteroaryl-C₃₋₈cycloalkyl- or heteroaryl-C₃₋₈heterocycloalkyl-, any of which may be optionally substituted;
—NR⁵R⁶ and —NR¹²R¹³ are each independently a linear structure, or, R⁵ and R⁶, or R¹² and R¹³, respectively, are taken together with the nitrogen atom to which they are attached to form a 3-12 membered saturated or unsaturated ring, wherein said ring optionally includes one or more additional heteroatoms selected from O, N, or S(O)ₘ₂;
—CR¹⁰R¹¹ and —CR¹⁴R¹⁵ are each independently a linear structure, or, R¹⁰ and R¹¹, or R¹⁴ and R¹⁵ respectively, are taken together with the carbon atom to which they are attached to form a 3-12 membered saturated or unsaturated ring, wherein said ring optionally includes one or more heteroatoms selected from O, N, or S(O)ₘ₃;
—CR¹⁹R²⁰ is a linear structure, or, R¹⁹ and R²⁰ are taken together with the carbon atom to which they are attached to form a 3-12 membered saturated or unsaturated ring, wherein said ring optionally includes one or more additional heteroatoms selected from O, N, or S(O)ₘ₄;
—NR¹⁷R¹⁸ is a linear structure, or, R¹⁷ and R¹⁸ are taken together with the nitrogen atom to which they are attached to form a 3-12 membered saturated or unsaturated ring, wherein said ring optionally includes one or more additional heteroatoms selected from O, N, or S(O)ₘ₅;
—CR²⁹R³⁰ is a linear structure, or, R²⁹ and R³⁰ are taken together with the carbon atom to which they are attached to form a 3-12 membered saturated or unsaturated ring, wherein said ring optionally includes one or more additional heteroatoms selected from O, N, or S(O)ₘ₆; and
—NR²⁷R²⁸ is a linear structure, or, R²⁷ and R²⁸ are taken together with the nitrogen atom to which they are attached to form a 3-12 membered saturated or unsaturated ring, wherein said ring optionally includes one or more additional heteroatoms selected from O, N, or S(O)ₘ₇;
wherein m1, m2, m3, m4, m5, m6, m7, n1, n2, n3, n4, n5 and n6 are each independently selected from 0, 1 or 2.

X¹ can be one or more of C₁₋₂ alkyl, C═O, NR³, or O. In some embodiments, X¹ can be C₁₋₂ alkyl or C═O. In some embodiments, X¹ can be C═O.

X² can be one or more of C₁₋₂ alkyl, C═O, NR³, or O. In some embodiments, X² can be NR³, or O. In some embodiments, X² can be NR³.

In some embodiments, R³ can be C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G³ substituents. In some embodiments, R³ is selected from C₀₋₁₂alkyl-, or C₃₋₁₂cycloalkyl-C₀₋₁₂alkyl-, any of which is optionally substituted with one or more independent G³ substituents. In some embodiments, R³ is selected from C₀₋₁₂alkyl-, or C₃₋₁₂cycloalkyl-C₀₋₁₂alkyl-, any of which is optionally substituted with one or more independent G³ substituents. In some embodiments, R³ is methyl, ethyl, propyl, butyl, pentyl, hexyl, cyclopropyl, cyclobutyl, cyclopentyl, cyclohexyl, or cyclopropylmethyl, any of which is optionally substituted with one or more independent G³ substituents. In some embodiments, R³ is methyl, ethyl, propyl, or cyclopropylmethyl, any of which is optionally substituted with one or more independent G³ substituents.

In some embodiments, R³ can be C₁₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G³ substituents. In some embodiments, R³ is selected from C₁₋₁₂alkyl-, or C₃₋₁₂cycloalkyl-C₀₋₁₂alkyl-, any of which is optionally substituted with one or more independent G³ substituents. In some embodiments, R³ is selected from C₁₋₁₂alkyl-, or C₃₋₁₂cycloalkyl-C₀₋₁₂alkyl-, any of which is optionally substituted with one or more independent G³ substituents.

In some embodiments, R³ is optionally substituted with one or more G³ substituents selected from H, halo, —CN, —CF₃, —OCF₃, —OCHF₂, —NR⁵R⁶, C₃₋₁₂cycloalkyl-C₀₋₁₂alkyl-, —S(O)ₙ₁R¹², —C(O)R¹², —C(O)NR¹²R¹³, —C(O)OR¹², —OC(O)R¹², —NR¹²C(O)R¹³, —NR¹²C(O)OR¹³, —NR¹²S(O)₂R¹³, or —(CR¹⁴R¹⁵)ₙ₁S(O)₂NR¹²R¹³. In some embodiments, R³ is optionally substituted with one or more G³ substituents selected from H, —CN, —NR⁵R⁶, C₃₋₁₂cycloalkyl-C₀₋₁₂alkyl-, —S(O)ₙ₁R¹², or —C(O)OR¹². In some embodiments, R³ is optionally substituted with one or more G³ substituents selected from —CN, —NMe₂, cyclopropyl, —SO₂Me, or —COOH. In some embodiments, R³ can be methyl, CH₂CN, CH₂-cyclopropyl, CH₂—COOH, CH₂CH₂CH₂—SO₂Me, or CH₂CH₂—NMe₂.

In some embodiments, R³ is selected from C₀₋₁₂alkyl-, or C₃₋₁₂cycloalkyl-C₀₋₁₂alkyl-, any of which is optionally substituted with one or more independent G³ substituents selected from H, halo, —CN, —CF₃, —OCF₃, —OCHF₂, —NR⁵R⁶, C₃₋₁₂cycloalkyl-C₀₋₁₂alkyl-, —S(O)ₙ₁R¹², —C(O)R¹², —C(O)NR¹²R¹³, —C(O)OR¹², —OC(O)R¹², —NR¹²C(O)R¹³, —NR¹²C(O)OR¹³, —NR¹²S(O)₂R¹³, or —(CR¹⁴R¹⁵)ₙ₁S(O)₂NR¹²R¹³. In some embodiments, R³ is selected from C₀₋₁₂alkyl-, or C₃₋₁₂cycloalkyl-C₀₋₁₂alkyl-, any of which is optionally substituted with one or more independent G³ substituents selected from H, —CN, —NR⁵R⁶, C₃₋₁₂cycloalkyl-C₀₋₁₂alkyl-, —S(O)ₙ₁R¹², or —C(O)OR¹². In some embodiments, R³ is methyl, ethyl, propyl, butyl, pentyl, hexyl, cyclopropyl, cyclobutyl, cyclopentyl, cyclohexyl, or cyclopropylmethyl, any of which is optionally substituted with one or more independent G³ substituents selected from —CN, —NMe₂, cyclopropyl, —SO₂Me, or —COOH. In some embodiments, R³ is methyl, ethyl, propyl, or cyclopropylmethyl, any of which is optionally substituted with one or more independent G³ substituents selected from —CN, —NMe₂, cyclopropyl, —SO₂Me, or —COOH.

In some embodiments, R³ is selected from C₁₋₁₂alkyl-, or C₃₋₁₂cycloalkyl-C₀₋₁₂alkyl-, any of which is optionally substituted with one or more independent G³ substituents selected from H, halo, —CN, —CF₃, —OCF₃, —OCHF₂, —NR⁵R⁶, C₃₋₁₂cycloalkyl-C₀₋₁₂alkyl-, —S(O)ₙ₁R¹², —C(O)R¹², —C(O)NR¹²R¹³, —C(O)OR¹², —OC(O)R¹², —NR¹²C(O)R¹³, —NR¹²C(O)OR¹³, —NR¹²S(O)₂R¹³, or —(CR¹⁴R¹⁵)ₙ₁S(O)₂NR¹²R¹³. In some embodiments, R³ is selected from C₁₋₁₂alkyl-, or C₃₋₁₂cycloalkyl-C₀₋₁₂alkyl-, any of which is optionally substituted with one or more independent G³ substituents selected from H, —CN, —NR⁵R⁶, C₃₋₁₂cycloalkyl-C₀₋₁₂alkyl-, —S(O)ₙ₁R¹², or —C(O)OR¹².

In some embodiments, X² can be NMe, N(CH₂CN), N(CH₂-cyclopropyl), N(CH₂—COOH), N(CH₂CH₂CH₂—SO₂Me), or N(CH₂CH₂—NMe₂). In some embodiments, X² can be NMe.

X³ can be one or more of C₁₋₂ alkyl, C═O, NR³, O, or CR¹⁰R¹¹. In some embodiments, X³ can be C₁₋₂ alkyl or C═O. In some embodiments, X³ can be CH₂ or C═O. In some embodiments, X³ can be CH₂. In some embodiments, X³ can be C═O.

In some embodiments, X¹ can be C₁₋₂ alkyl or C═O, X² can be NR³, or O, and X³ can be C₁₋₂ alkyl or C═O. In some embodiments, X¹ can be C═O, X² can be NR³, or O, and X³ can be C₁₋₂ alkyl or C═O. In some embodiments, X¹ can be C═O, X² can be NR³, and X³ can be C₁₋₂ alkyl or C═O. In some embodiments, X¹ can be C═O, X² can be NR³, and X³ can be C═O. In some embodiments, X¹ can be C═O, X² can be NR³, and X³ can be C₁₋₂ alkyl. In some embodiments, X¹ can be C═O, X² can be NMe, and X³ can be C═O. In some embodiments, X¹ can be C═O, X² can be NMe, and X³ can be C₁₋₂ alkyl. In some embodiments, X¹ can be C═O, X² can be NMe, and X³ can be CH₂.

Subscripts m and n are each independently selected from 0, 1 or 2. In some embodiments, m and n are each 1.

In some embodiments, R¹ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G¹ substituents. In some embodiments, R¹ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G¹ substituents. In some embodiments, R¹ is selected from C₀₋₁₂alkyl-, C₃₋₁₂cycloalkyl-C₀₋₁₂alkyl-, aryl-C₀₋₁₂alkyl-, or heteroaryl-C₀₋₁₂alkyl-, any of which is optionally substituted with one or more independent G¹ substituents. In some embodiments, R¹ is selected from methyl, ethyl, propyl, n-propyl, i-propyl, butyl, sec-butyl, iso-butyl, tert-butyl, pentyl, hexyl, cyclopropyl, cyclobutyl, cyclopentyl, cyclohexyl, adamantyl, phenyl, pyrrole, pyrazole, imidazole, triazole, tetrazole, furan, thiophene, oxazole, isoxazole, thiazole, isothiazole, oxadiazole, thiadiazole, dithiazole, pyridine, pyrazine, pyrimidine, pyridazine, triazine, tetrazine, pyran, dioxine, trioxane, dithiine, trithiane, thiopyran, oxazine, or thiazine, each optionally substituted with one or more independent G¹ substituents. In some embodiments, R¹ is selected from t-Bu, cyclohexane, adamantyl, phenyl, pyridine or thiazole, each optionally substituted with one or more independent G¹ substituents.

In some embodiments, R¹ is optionally substituted with one or more independent G¹ substituents selected from H, halo, —CN, —CF₃, —OCF₃, —OCHF₂, —NR⁵R⁶, —NO₂, —C₀₋₁₂alkyl, —C₂₋₁₂alkenyl, —C₂₋₁₂alkynyl, C₃₋₁₂cycloalkyl-C₀₋₁₂alkyl-, C₃₋₁₂heterocycloalkyl-C₀₋₁₂alkyl-, aryl-C₀₋₁₂alkyl-, heteroaryl-C₀₋₁₂alkyl-, —OC₀₋₁₂alkyl, —S(O)ₙ₁R¹², —C(O)R¹², —C(O)NR¹²R¹³, C(O)OR¹², —OC(O)R¹², —NR¹²C(O)R¹³, —NR¹²C(O)OR¹³, —NR¹²S(O)₂R¹³, or —(CR¹⁴R¹⁵)ₙ₁S(O)₂NR¹²R¹³. In some embodiments, R¹ is optionally substituted with one or more independent G¹ substituents selected from H, halo, —CN, —CF₃, —OCF₃, —OCHF₂, —C₀₋₁₂alkyl, C₃₋₁₂cycloalkyl-C₀₋₁₂alkyl-, or —OC₀₋₁₂alkyl. In some embodiments, R¹ is optionally substituted with one or more independent G¹ substituents selected from H, F, Cl, —CN, —CF₃, —OCF₃, —OCHF₂, —OCH₂F, methyl, ethyl, propyl, n-propyl, i-propyl, butyl, sec-butyl, iso-butyl, tert-butyl, pentyl, hexyl, cyclopropyl, cyclobutyl, cyclopentyl, cyclohexyl, or methoxy. In some embodiments, R¹ is optionally substituted with one or more independent G¹ substituents selected from H, F, Cl, —CN, —CF₃, —OCF₃, —OCH₂F, methyl, ethyl, cyclopropyl, or methoxy.

In some embodiments, R¹ is selected from C₀₋₁₂alkyl-, C₃₋₁₂cycloalkyl-C₀₋₁₂alkyl-, aryl-C₀₋₁₂alkyl-, or heteroaryl-C₀₋₁₂alkyl-, any of which is optionally substituted with one or more independent G¹ substituents selected from H, halo, —CN, —CF₃, —OCF₃, —OCHF₂, —C₀₋₁₂alkyl, C₃₋₁₂cycloalkyl-C₀₋₁₂alkyl-, or —OC₀₋₁₂alkyl. In some embodiments, R¹ is selected from methyl, ethyl, propyl, n-propyl, i-propyl, butyl, sec-butyl, iso-butyl, tert-butyl, pentyl, hexyl, cyclopropyl, cyclobutyl, cyclopentyl, cyclohexyl, adamantyl, phenyl, pyrrole, pyrazole, imidazole, triazole, tetrazole, furan, thiophene, oxazole, isoxazole, thiazole, isothiazole, oxadiazole, thiadiazole, dithiazole, pyridine, pyrazine, pyrimidine, pyridazine, triazine, tetrazine, pyran, dioxine, trioxane, dithiine, trithiane, thiopyran, oxazine, or thiazine, each optionally substituted with one or more independent G¹ substituents selected from H, F, Cl, —CN, —CF₃, —OCF₃, —OCHF₂, —OCH₂F, methyl, ethyl, propyl, n-propyl, i-propyl, butyl, sec-butyl, iso-butyl, tert-butyl, pentyl, hexyl, cyclopropyl, cyclobutyl, cyclopentyl, cyclohexyl, or methoxy. In some embodiments, R¹ is selected from t-Bu, cyclohexane, adamantyl, phenyl, pyridine or thiazole, each optionally substituted with one or more independent G¹ substituents selected from H, F, Cl, —CN, —CF₃, —OCF₃, —OCH₂F, methyl, ethyl, cyclopropyl, or methoxy.

In some embodiments, X¹ can be C₁₋₂ alkyl or C═O, X² can be NR³, or O, X³ can be C₁₋₂ alkyl or C═O, m and n are each 1, R¹ is selected from C₀₋₁₂alkyl-, C₃₋₁₂cycloalkyl-C₀₋₁₂alkyl-, aryl-C₀₋₁₂alkyl-, or heteroaryl-C₀₋₁₂alkyl-, any of which is optionally substituted with one or more independent G¹ substituents selected from H, halo, —CN, —CF₃, —OCF₃, —OCHF₂, —C₀₋₁₂alkyl, C₃₋₁₂cycloalkyl-C₀₋₁₂alkyl-, or —OC₀₋₁₂alkyl, and R³ is selected from C₀₋₁₂alkyl-, or C₃₋₁₂cycloalkyl-C₀₋₁₂alkyl-, any of which is optionally substituted with one or more independent G³ substituents selected from H, —CN, —NR⁵R⁶, C₃₋₁₂cycloalkyl-C₀₋₁₂alkyl-, —S(O)ₙ₁R¹², or —C(O)OR¹².

In some embodiments, R² is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G² substituents. In some embodiments, R² is selected from C₀₋₁₂alkyl-, C₃₋₁₂cycloalkyl-C₀₋₁₂alkyl-,C₃₋₁₂heterocycloalkyl-C₀₋₁₂alkyl-, aryl-C₀₋₁₂alkyl-, or heteroaryl-C₀₋₁₂alkyl-, any of which is optionally substituted with one or more independent G² substituents. In some embodiments, R² is selected from C₀₋₁₂alkyl-, C₃₋₁₂cycloalkyl-C₀₋₁₂alkyl-, or C₃₋₁₂heterocycloalkyl-C₀₋₁₂alkyl-, any of which is optionally substituted with one or more independent G² substituents. In some embodiments, R² is selected from H, methyl, ethyl, propyl, n-propyl, i-propyl, butyl, sec-butyl, iso-butyl, tert-butyl, pentyl, hexyl, cyclopropyl, cyclobutyl, cyclopentyl, cyclohexyl, or tetrahydro-2H-pyran, wherein the ethyl group is optionally substitued with one or more G² substituents. In some embodiments, R² is selected from H, methyl, ethyl, isopropyl, sec-butyl, cyclopropyl, cyclobutyl, cyclopentyl, cyclohexyl, or tetrahydro-2H-pyran, wherein the ethyl group is optionally substitued with one or more G² substituents.

In some embodiments, R² is optionally substituted with one or more independent G² substituents selected from H, halo, —CN, —CF₃, —OCF₃, —OCHF₂, —NR⁵R⁶, —NO₂, —C₀₋₁₂alkyl, —C₂₋₁₂alkenyl, —C₂₋₁₂alkynyl, C₃₋₁₂cycloalkyl-C₀₋₁₂alkyl-, C₃₋₁₂heterocycloalkyl-C₀₋₁₂alkyl-, aryl-C₀₋₁₂alkyl-, heteroaryl-C₀₋₁₂alkyl-, —OC₀₋₁₂alkyl, —S(O)ₙ₁R¹², —C(O)R¹², —C(O)NR¹²R¹³, C(O)OR¹², —OC(O)R¹², —NR¹²C(O)R¹³, —NR¹²C(O)OR¹³, —NR¹²S(O)₂R¹³, or —(CR¹⁴R¹⁵)ₙ₁S(O)₂NR¹²R¹³. In some embodiments, R² is optionally substituted with one or more independent G² substituents selected from H or —OC₀₋₁₂alkyl. In some embodiments, R² is optionally substitued with one or more G² substituent selected from —OMe;

In some embodiments, R² is selected from C₀₋₁₂alkyl-, C₃₋₁₂cycloalkyl-C₀₋₁₂alkyl-,C₃₋₁₂heterocycloalkyl-C₀₋₁₂alkyl-, aryl-C₀₋₁₂alkyl-, or heteroaryl-C₀₋₁₂alkyl-, any of which is optionally substituted with one or more independent G² substituents selected from H, halo, —CN, —CF₃, —OCF₃, —OCHF₂, —NR⁵R⁶, —NO₂, —C₀₋₁₂alkyl, —C₂₋₁₂alkenyl, —C₂₋₁₂alkynyl, C₃₋₁₂cycloalkyl-C₀₋₁₂alkyl-, C₃₋₁₂heterocycloalkyl-C₀₋₁₂alkyl-, aryl-C₀₋₁₂alkyl-, heteroaryl-C₀₋₁₂alkyl-, —OC₀₋₁₂alkyl, —S(O)ₙ₁R¹², —C(O)R¹², —C(O)NR¹²R¹³, —C(O)OR¹², —OC(O)R¹², —NR¹²C(O)R¹³, —NR¹²C(O)OR¹³, —NR¹²S(O)₂R¹³, or —(CR¹⁴R¹⁵)ₙ₁S(O)₂NR¹²R¹³. In some embodiments, R² is selected from C₀₋₁₂alkyl-, C₃₋₁₂cycloalkyl-C₀₋₁₂alkyl-, or C₃₋₁₂heterocycloalkyl-C₀₋₁₂alkyl-, any of which is optionally substituted with one or more independent G² substituents selected from H or —OC₀₋₁₂alkyl. In some embodiments, R² is selected from H, methyl, ethyl, propyl, n-propyl, i-propyl, butyl, sec-butyl, iso-butyl, tert-butyl, pentyl, hexyl, cyclopropyl, cyclobutyl, cyclopentyl, cyclohexyl, or tetrahydro-2H-pyran, wherein the ethyl group is optionally substitued with one or more G² substituent selected from —OMe. In some embodiments, R² is selected from H, methyl, ethyl, isopropyl, sec-butyl, cyclopropyl, cyclobutyl, cyclopentyl, cyclohexyl, or tetrahydro-2H-pyran, wherein the ethyl group is optionally substitued with one or more G² substituent selected from —OMe.

In some embodiments, X¹ can be C₁₋₂ alkyl or C═O, X² can be NR³, or O, X³ can be C₁₋₂ alkyl or C═O, m and n are each 1, R¹ is selected from C₀₋₁₂alkyl-, C₃₋₁₂cycloalkyl-C₀₋₁₂alkyl-, aryl-C₀₋₁₂alkyl-, or heteroaryl-C₀₋₁₂alkyl-, any of which is optionally substituted with one or more independent G¹ substituents selected from H, halo, —CN, —CF₃, —OCF₃, —OCHF₂, —C₀₋₁₂alkyl, C₃₋₁₂cycloalkyl-C₀₋₁₂alkyl-, or —OC₀₋₁₂alkyl, R² is selected from C₀₋₁₂alkyl-, C₃₋₁₂cycloalkyl-C₀₋₁₂alkyl-, or C₃₋₁₂heterocycloalkyl-C₀₋₁₂alkyl-, any of which is optionally substituted with one or more independent G² substituents selected from H or —OC₀₋₁₂alkyl, and R³ is selected from C₀₋₁₂alkyl-, or C₃₋₁₂cycloalkyl-C₀₋₁₂alkyl-, any of which is optionally substituted with one or more independent G³ substituents selected from H, —CN, —NR⁵R⁶, C₃₋₁₂cycloalkyl-C₀₋₁₂alkyl-, —S(O)ₙR¹², or —C(O)OR¹².

In some embodiments, R^{2a} is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G^{2a} substituents. In some embodiments, R^{2a} is selected from C₀₋₁₂alkyl-, or C₃₋₁₂heterocycloalkyl-C₀₋₁₂alkyl-. In some embodiments, R^{2a} is selected from H, aziridine, azetidine, pyrrolidine, imidazolidine, pyrazolidine, piperidine, piperazine, triazine, tetrazine, oxirane, oxetane, tetrahydrofuran, oxane, dioxane, trioxane, thiirane, thietane, tetrahydrothiophene, ditholane, thiane, dithiane, trithiane, oxaziridine, dioxirane, oxazolidine, isoxazolidine, thiazolidine, isothiazolidine, morpholine, or thiomorpholine. In some embodiments, R^{2a} is selected from H or tetrahydro-2H-pyran.

In some embodiments, R² and R^{2a} are each independently a linear structure, or, R² and R^{2a} are taken together with the carbon atom to which they are attached to form a 3-12 membered saturated or unsaturated ring, wherein said ring optionally includes one or more additional heteroatoms selected from O, N, or S(O)ₘ₁. In some embodiments, R² and R^{2a} are taken together with the carbon atom to which they are attached to form a cyclopropyl, cyclobutyl, cyclopentyl, cyclohexyl, aziridine, azetidine, pyrrolidine, imidazolidine, pyrazolidine, piperidine, piperazine, triazine, tetrazine, oxirane, oxetane, tetrahydrofuran, oxane, dioxane, trioxane, thiirane, thietane, tetrahydrothiophene, ditholane, thiane, dithiane, trithiane, oxaziridine, dioxirane, oxazolidine, isoxazolidine, thiazolidine, isothiazolidine, morpholine, or thiomorpholine. In some embodiments, R² and R^{2a} are taken together with the carbon atom to which they are attached to form a cyclopropane or oxetane.

In some embodiments, X¹ can be C₁₋₂ alkyl or C═O, X² can be NR³, or O, X³ can be C₁₋₂ alkyl or C═O, m and n are each 1, R¹ is selected from C₀₋₁₂alkyl-, C₃₋₁₂cycloalkyl-C₀₋₁₂alkyl-, aryl-C₀₋₁₂alkyl-, or heteroaryl-C₀₋₁₂alkyl-, any of which is optionally substituted with one or more independent G¹ substituents selected from H, halo, —CN, —CF₃, —OCF₃, —OCHF₂, —C₀₋₁₂alkyl, C₃₋₁₂cycloalkyl-C₀₋₁₂alkyl-, or —OC₀₋₁₂alkyl, R² is selected from C₀₋₁₂alkyl-, C₃₋₁₂cycloalkyl-C₀₋₁₂alkyl-, or C₃₋₁₂heterocycloalkyl-C₀₋₁₂alkyl-, any of which is optionally substituted with one or more independent G² substituents selected from H or —OC₀₋₁₂alkyl, R^{2a} is selected from C₀₋₁₂alkyl-, or C₃₋₁₂heterocycloalkyl-C₀₋₁₂alkyl-, and R³ is selected from C₀₋₁₂alkyl-, or C₃₋₁₂cycloalkyl-C₀₋₁₂alkyl-, any of which is optionally substituted with one or more independent G³ substituents selected from H, —CN, —NR⁵R⁶, C₃₋₁₂cycloalkyl-C₀₋₁₂alkyl-, —S(O)ₙ₁R¹², or —C(O)OR¹².

In some embodiments, R⁴ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, heteroaryl-C₃₋₁₂heterocycloalkyl-, or pyridine-N-oxide, any of which is optionally substituted with one or more independent G⁴ substituents. In some embodiments, R⁴ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, heteroaryl-C₀₋₁₂alkyl-, or pyridine-N-oxide, any of which is optionally substituted with one or more independent G⁴ substituents. In some embodiments, R⁴ is selected from H, methyl, ethyl, isopentyl, cyclopropyl, cyclopropylmethyl, cyclobutylmethyl, cyclopentyl, phenyl, phenylethyl, benzyl, benzofuryl, (azetidine)methyl, 1H-benzo[d]imidazole, (1H-benzo[d]imidazole)methyl, benzo[d]oxazole, (benzo[d]oxazole)methyl, benzo[d][1,3]dioxole, 1H-benzo[d][1,2,3]triazole, (2,3-dihydrobenzo[b][1,4] dioxin)methyl, 3,4-dihydro-2H-benzo[b][1,4]oxazine, 2,3-dihydrobenzo[d]oxazole, (2,3-dihydrobenzo[d]oxazole)methyl, 1,6-dihydropyridine, (1,6-dihydropyridine)methyl, 3,4-dihydro-2H-benzo[b][1,4]oxazine, imidazo[1,2-a]pyridine, imidazo[1,2-a]pyridine-methyl, imidazo[1,5-a]pyridine, 1H-indazole, (1H-indazole)methyl, 1H-indole, 1H-pyrazole, (1H-pyrazole)methyl, pyridine, pyridine-methyl, pyrimidine-methyl, 1H-pyrrolo[2,3-b]pyridine, quinoxaline-methyl, tetrahydro-2H-pyran, (thiazole)methyl, or ([1,2,4]triazolo[1,5-a]pyridine)methyl, any of which is optionally substituted with one or more independent G⁴ substituents. In some embodiments, R⁴ is selected from phenyl and 1H-indazole, any of which is optionally substituted with one or more independent G⁴ substituents.

In some embodiments, each R⁴ is optionally substituted with one or more independent G⁴ substituents selected from H, D, halo, —CN, —CD₃, —OCD₃, -oxo-, —CF₃, —OCF₃, —OCHF₂, —NR⁵R⁶, —NO₂, —B(OH)₂, —PO(OR¹²)₂, —PO(OR¹²)R¹³, —CONR¹²OH, —C₀₋₁₂alkyl, —C₂₋₁₂alkenyl, —C₂₋₁₂alkynyl, C₃₋₁₂cycloalkyl-C₀₋₁₂alkyl-, C₃₋₁₂heterocycloalkyl-C₀₋₁₂alkyl-, aryl-C₀₋₁₂alkyl-, heteroaryl-C₀₋₁₂alkyl-, —OC₀₋₁₂alkyl, —S(O)ₙ₁R¹², C(O)R¹², —C(O)NR¹²R¹³, —C(O)OR¹², —OC(O)R¹², —NR¹²C(O)R¹³, —NR¹²C(O)OR¹³, —NR¹²S(O)₂R²S(O)₂R¹³, or —(CR¹⁴R¹⁵)ₙ₁S(O)₂NR¹²R¹³ optionally substituted with one or more independent Q¹ substituents. In some embodiments, each R⁴ is optionally substituted with one or more independent G⁴ substituents selected from H, D, halo, —CN, —OCD₃, -oxo-, —CF₃, —OCF₃, —NR⁵R⁶, —B(OH)₂, —C₀₋₁₂alkyl, aryl-C₀₋₁₂alkyl-, heteroaryl-C₀₋₁₂alkyl-, —OC₀₋₁₂alkyl, —C(O)R¹², —S(O)ₙ₁R¹², —C(O)NR¹²R¹³, —C(O)OR¹², —NR¹²C(O)R¹³, —NR¹²C(O)OR¹³, or —NR¹²S(O)₂R¹³, optionally substituted with one or more independent Q¹ substituents. In some embodiments, each R⁴ is optionally substituted with one or more independent G⁴ substituents selected from H, D, F, Cl, Br, —CN, —OCD₃, oxo, —CF₃, —OCF₃, —NH(azetidine), —NH(oxetane), —B(OH)₂, Me, triazole, tetrazole, —OMe, -OEt, —SO₂Me, —C(O)NH₂, —COOH, —C(O)OMe, —NHC(O)-cyclopropane, —NHC(O)OMe, or —NHSO₂Me, optionally substituted with one or more independent Q¹ substituents.

In some embodiments, each G⁴ substituent is optionally substituted with one or more independent Q¹ substituents selected from halo, —CN, —CF₃, —OCF₃, —OCHF₂, NR¹⁷R¹⁸, —C₀₋₁₂alkyl, aryl-C₀₋₁₂alkyl-, heteroaryl-C₀₋₁₂alkyl-, C₃₋₁₂cycloalkyl-C₀₋₁₂alkyl-, C₃₋₁₂heterocycloalkyl-C₀₋₁₂alkyl-, —OC₀₋₁₂alkyl, —OC(O)R¹⁷, —NR¹⁷C(O)R, —NR¹⁷S(O)₂R¹⁸, —(CR¹⁹R²⁰)ₙ₃C(O)R¹⁷, —(CR¹⁹R²⁰)ₙ₃C(O)OR¹⁷, —(CR¹⁹R²⁰)ₙ₃C(O)NR¹⁷R¹⁸, —(CR¹⁹R²⁰)ₙ₃S(O)₂NR¹⁷R¹⁸, —(CR¹⁹R²⁰)ₙ₃NR¹⁷R¹⁸, or —(CR¹⁹R²⁰)ₙ₃OR¹⁷. In some embodiments, each G⁴ substituent is optionally substituted with one or more independent Q¹ substituents selected from —CN, NR¹⁷R¹⁸, C₀₋₁₂alkyl-, C₃₋₁₂heterocycloalkyl-C₀₋₁₂alkyl-, —OC₀₋₁₂alkyl, —(CR¹⁹R²⁰)ₙ₃C(O)OR¹⁷, or —(CR¹⁹R²⁰)ₙ₃C(O)NR¹⁷R¹⁸. In some embodiments, each G⁴ substituent is optionally substituted with one or more independent Q¹ substituents selected from —CN, NMe₂, Me, azetidine, oxetane, —OH, —COOH, or —C(O)NH₂.

In some embodiments, R⁴ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, heteroaryl-C₃₋₁₂heterocycloalkyl-, or pyridine-N-oxide, any of which is optionally substituted with one or more independent G⁴ substituents selected from H, D, halo, —CN, —CD₃, —OCD₃, -oxo-, —CF₃, —OCF₃, —OCHF₂, —NR⁵R⁶, —NO₂, —B(OH)₂, —PO(OR¹²)₂, —PO(OR¹²)R¹³, —CONR¹²OH, —C₀₋₁₂alkyl, —C₂₋₁₂alkenyl, —C₂₋₁₂alkynyl, C₃₋₁₂cycloalkyl-C₀₋₁₂alkyl-, C₃₋₁₂heterocycloalkyl-C₀₋₁₂alkyl-, aryl-C₀₋₁₂alkyl-, heteroaryl-C₀₋₁₂alkyl-, —OC₀₋₁₂alkyl, —S(O)ₙ₁R¹², C(O)R¹², —C(O)NR¹²R¹³, —C(O)OR¹², —OC(O)R¹², —NR¹²C(O)R¹³, —NR¹²C(O)OR¹³, —NR¹²S(O)₂R¹³, or —(CR¹⁴R¹⁵)ₙ₁S(O)₂NR¹²R¹³, optionally substituted with one or more independent Q¹ substituents selected from halo, —CN, —CF₃, —OCF₃, —OCHF₂, NR¹⁷R¹⁸, —C₀₋₁₂alkyl, aryl-C₀₋₁₂alkyl-, heteroaryl-C₀₋₁₂alkyl-, C₃₋₁₂cycloalkyl-C₀₋₁₂alkyl-, C₃₋₁₂heterocycloalkyl-C₀₋₁₂alkyl-, —OC₀₋₁₂alkyl, —OC(O)R¹⁷, —NR¹⁷C(O)R¹⁸, —NR¹⁷S(O)₂R¹⁸, —(CR¹⁹R²⁰)ₙ₃C(O)R¹⁷, —(CR¹⁹R²⁰)ₙ₃C(O)OR¹⁷, —(R¹⁹R²⁰)ₙ₃C(O)NR¹⁷R¹⁸, —(CR¹⁹R²⁰)ₙ₃S(O)₂NR¹⁷R¹⁸, (CR¹⁹R²⁰)ₙ₃NR¹⁷R¹⁸, or —(CR¹⁹R²⁰)ₙ₃OR¹⁷. In some embodiments, R⁴ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, heteroaryl-C₀₋₁₂alkyl-, or pyridine-N-oxide, any of which is optionally substituted with one or more independent G⁴ substituents selected from H, D, halo, —CN, —OCD₃, -oxo-, —CF₃, —OCF₃, —NR⁵R⁶, —B(OH)₂, —C₀₋₁₂alkyl, aryl-C₀₋₁₂alkyl-, heteroaryl-C₀₋₁₂alkyl-, —OC₀₋₁₂alkyl, —C(O)R¹², —S(O)ₙ₁R¹², —C(O)NR¹²R¹³, —C(O)OR¹², —NR¹²C(O)R¹³, —NR¹²C(O)OR¹³, or —NR¹²S(O)₂R¹³ optionally substituted with one or more independent Q¹ substituents selected from —CN, NR¹⁷R¹⁸, C₀₋₁₂alkyl-, C₃₋₁₂heterocycloalkyl-C₀₋₁₂alkyl-, —OC₀₋₁₂alkyl, —(CR¹⁹R²⁰)ₙ₃C(O)OR¹⁷, or —(CR¹⁹R²⁰)ₙ₃C(O)NR¹⁷R¹⁸. In some embodiments, R⁴ is selected from H, methyl, ethyl, isopentyl, cyclopropyl, cyclopropylmethyl, cyclobutylmethyl, cyclopentyl, phenyl, phenylethyl, benzyl, benzofuryl, (azetidine)methyl, 1H-benzo[d]imidazole, (1H-benzo[d]imidazole)methyl, benzo[d]oxazole, (benzo[d]oxazole)methyl, benzo[d][1,3]dioxole, 1H-benzo[d][1,2,3]triazole, (2,3-dihydrobenzo[b][1,4] dioxin)methyl, 3,4-dihydro-2H-benzo[b][1,4]oxazine, 2,3-dihydrobenzo[d]oxazole, (2,3-dihydrobenzo[d]oxazole)methyl, 1,6-dihydropyridine, (1,6-dihydropyridine)methyl, 3,4-dihydro-2H-benzo[b][1,4]oxazine, imidazo[1,2-a]pyridine, imidazo[1,2-a]pyridine-methyl, imidazo[1,5-a]pyridine, 1H-indazole, (1H-indazole)methyl, 1H-indole, 1H-pyrazole, (1H-pyrazole)methyl, pyridine, pyridine-methyl, pyrimidine-methyl, 1H-pyrrolo[2,3-b]pyridine, quinoxaline-methyl, tetrahydro-2H-pyran, (thiazole)methyl, or ([1,2,4]triazolo[1,5-a]pyridine)methyl, any of which is optionally substituted with one or more independent G⁴ substituents selected from H, D, F, Cl, Br, —CN, —OCD₃, oxo, —CF₃, —OCF₃, —NH(azetidine), —NH(oxetane), —B(OH)₂, Me, triazole, tetrazole, —OMe, -OEt, —SO₂Me, —C(O)NH₂, —COOH, —C(O)OMe, —NHC(O)-cyclopropane, —NHC(O)OMe, or —NHSO₂Me, optionally substituted with one or more independent Q¹ substituents selected from —CN, NMe₂, Me, azetidine, oxetane, —OH, —COOH, or —C(O)NH₂.

In some embodiments, R⁴ is selected from phenyl and 1H-indazole, each of which is optionally substituted with one or more independent G⁴ substituents selected from H, D, F, Cl, Br, —CN, —OCD₃, oxo, —CF₃, —OCF₃, —NH(azetidine), —NH(oxetane), —B(OH)₂, Me, triazole, tetrazole, —OMe, -OEt, —SO₂Me, —C(O)NH₂, —COOH, —C(O)OMe, —NHC(O)-cyclopropane, —NHC(O)OMe, or —NHSO₂Me, optionally substituted with one or more independent Q¹ substituents selected from —CN, NMe₂, Me, azetidine, oxetane, —OH, —COOH, or —C(O)NH₂.

In some embodiments, X¹ can be C₁₋₂ alkyl or C═O, X² can be NR³, or O, X³ can be C₁₋₂ alkyl or C═O, m and n are each 1, R¹ is selected from C₀₋₁₂alkyl-, C₃₋₁₂cycloalkyl-C₀₋₁₂alkyl-, aryl-C₀₋₁₂alkyl-, or heteroaryl-C₀₋₁₂alkyl-, any of which is optionally substituted with one or more independent G¹ substituents selected from H, halo, —CN, —CF₃, —OCF₃, —OCHF₂, —C₀₋₁₂alkyl, C₃₋₁₂cycloalkyl-C₀₋₁₂alkyl-, or —OC₀₋₁₂alkyl, R² is selected from C₀₋₁₂alkyl-, C₃₋₁₂cycloalkyl-C₀₋₁₂alkyl-, or C₃₋₁₂heterocycloalkyl-C₀₋₁₂alkyl-, any of which is optionally substituted with one or more independent G² substituents selected from H or —OC₀₋₁₂alkyl, R^{2a} is selected from C₀₋₁₂alkyl-, or C₃₋₁₂heterocycloalkyl-C₀₋₁₂alkyl-, R³ is selected from C₀₋₁₂alkyl-, or C₃₋₁₂cycloalkyl-C₀₋₁₂alkyl-, any of which is optionally substituted with one or more independent G³ substituents selected from H, —CN, —NR⁵R⁶, C₃₋₁₂cycloalkyl-C₀₋₂alkyl-, —S(O)ₙ₁R¹², or —C(O)OR¹², and R⁴ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, heteroaryl-C₀₋₁₂alkyl-, or pyridine-N-oxide, any of which is optionally substituted with one or more independent G⁴ substituents selected from H, D, halo, —CN, —OCD₃, -oxo-, —CF₃, —OCF₃, —NR⁵R⁶, —B(OH)₂, —C₀₋₁₂alkyl, aryl-C₀₋₁₂alkyl-, heteroaryl-C₀₋₁₂alkyl-, —OC₀₋₁₂alkyl, —C(O)R¹², —S(O)ₙ₁R¹², —C(O)NR¹²R¹³, —C(O)OR¹², —NR¹²C(O)R¹³, —NR¹²C(O)OR¹³, or —NR¹²S(O)₂R¹³, optionally substituted with one or more independent Q¹ substituents selected from —CN, NR¹⁷R¹⁸, C₀₋₁₂alkyl-, C₃₋₁₂heterocycloalkyl-C₀₋₁₂alkyl-, —OC₀₋₁₂alkyl, —(CR¹⁹R²⁰)ₙ₃C(O)OR¹⁷, or —(CR¹⁹R²⁰)ₙ₃C(O)NR¹⁷R¹⁸.

In some embodiments, the compounds of Formula I are those wherein
X¹ is selected from C₁₋₂ alkyl and C═O;
X² is selected from NR³ and O;
X³ is independently selected from one or more of C₁₋₂ alkyl or C═O;
m and n are each independently selected from 0, 1 or 2;
R¹ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G¹ substituents selected from H, halo, —CN, —CF₃, —OCF₃, —OCHF₂, —NR⁵R⁶, —NO₂, —C₀₋₁₂alkyl, —C₂₋₁₂alkenyl, —C₂₋₁₂alkynyl, C₃₋₁₂cycloalkyl-C₀₋₁₂alkyl-, C₃₋₁₂heterocycloalkyl-C₀₋₁₂alkyl-, aryl-C₀₋₁₂alkyl-, heteroaryl-C₀₋₁₂alkyl-, —OC₀₋₁₂alkyl, —S(O)ₙ₁R¹², —C(O)R¹², —C(O)NR¹²R¹³, —C(O)OR¹², —OC(O)R¹², —NR¹²C(O)R¹³, —NR¹²C(O)OR¹³, —NR¹²S(O)₂R¹³, or —(CR¹⁴R¹⁵)ₙ₁S(O)₂NR¹²R¹³;
R² is selected from C₀₋₁₂alkyl-, C₃₋₁₂cycloalkyl-C₀₋₁₂alkyl-,C₃₋₁₂heterocycloalkyl-C₀₋₁₂alkyl-, aryl-C₀₋₁₂alkyl-, or heteroaryl-C₀₋₁₂alkyl-, any of which is optionally substituted with one or more independent G² substituents selected from H, halo, —CN, —CF₃, —OCF₃, —OCHF₂, —NR⁵R⁶, —NO₂, —C₀₋₁₂alkyl, —C₂₋₁₂alkenyl, —C₂₋₁₂alkynyl, C₃₋₁₂cycloalkyl-C₀₋₁₂alkyl-, C₃₋₁₂heterocycloalkyl-C₀₋₁₂alkyl-, aryl-C₀₋₁₂alkyl-, heteroaryl-C₀₋₁₂alkyl-, —OC₀₋₁₂alkyl, —S(O)ₙ₁R¹², —C(O)R¹², —C(O)NR¹²R¹³, —C(O)OR¹², —OC(O)R¹², —NR¹²C(O)R¹³, —NR¹²C(O)OR¹³, NR¹²S(O)₂R¹³, or —(CR¹⁴R¹⁵)ₙ₁S(O)₂NR¹²R¹³;
R^{2a} is selected from C₀₋₁₂alkyl-, or C₃₋₁₂heterocycloalkyl-C₀₋₁₂alkyl-;
R² and R^{2a} are each independently a linear structure, or, R² and R^{2a} are taken together with the carbon atom to which they are attached to form a 3-12 membered saturated or unsaturated ring, wherein said ring optionally includes one or more additional heteroatoms selected from O, N, or S(O)ₘ₁;
R³ is selected from C₀₋₁₂alkyl-, or C₃₋₁₂cycloalkyl-C₀₋₁₂alkyl-, optionally substituted with one or more independent G³ substituents selected from H, halo, —CN, —CF₃, —OCF₃, —OCHF₂, —NR⁵R⁶, C₃₋₁₂cycloalkyl-C₀₋₁₂alkyl-, —S(O)ₙ₁R¹², —C(O)R¹², —C(O)NR¹²R¹³, —C(O)OR¹², —OC(O)R¹², —NR¹²C(O)R¹³, —NR¹²C(O)OR¹³, NR¹²S(O)₂R¹³, or —(CR¹⁴R¹⁵)ₙ₁S(O)₂NR¹²R¹³;
R⁴ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, heteroaryl-C₃₋₁₂heterocycloalkyl-, or pyridine-N-oxide, any of which is optionally substituted with one or more independent G⁴ substituents selected from H, D, halo, —CN, —CD₃, —OCD₃, -oxo-, —CF₃, —OCF₃, —OCHF₂, —NR⁵R⁶, —NO₂, —B(OH)₂, —PO(OR¹²)₂, —PO(OR¹²)R¹³, —CONR¹²OH, —C₀₋₁₂alkyl, —C₂₋₁₂alkenyl, —C₂₋₁₂alkynyl, C₃₋₁₂cycloalkyl-C₀₋₁₂alkyl-, C₃₋₁₂heterocycloalkyl-C₀₋₁₂alkyl-, aryl-C₀₋₁₂alkyl-, heteroaryl-C₀₋₁₂alkyl-, —OC₀₋₁₂alkyl, —S(O)ₙ₁R¹², C(O)R¹², —C(O)NR¹²R¹³, —C(O)OR¹², —OC(O)R¹², —NR¹²C(O)R¹³, —NR¹²C(O)OR¹³, —NR¹²S(O)₂R¹³, or —(CR¹⁴R¹⁵)ₙ₁S(O)₂NR¹²R¹³, wherein —OC₀₋₁₂alkyl is optionally substituted with one or more independent Q¹ substituents selected from halo, —CN, —CF₃, —OCF₃, —OCHF₂, NR¹⁷R¹⁸, —C₀₋₁₂alkyl, aryl-C₀₋₁₂alkyl-, heteroaryl-C₀₋₁₂alkyl-, C₃₋₁₂cycloalkyl-C₀₋₁₂alkyl-, C₃₋₁₂heterocycloalkyl-C₀₋₁₂alkyl-, —OC₀₋₁₂alkyl, —OC(O)R¹⁷, —NR¹⁷C(O)R¹⁸, —NR¹⁷S(O)₂R¹⁸, —(CR¹⁹R²⁰)ₙ₃C(O)R¹⁷, —(CR¹⁹R²⁰)ₙ₃C(O)OR¹⁷, —(CR¹⁹R²⁰)ₙ₃C(O)NR¹⁷R¹⁸, —(CR¹⁹R²⁰)ₙ₃S(O)₂NR¹⁷R¹⁸, —(CR¹⁹R²⁰)ₙ₃NR¹⁷R¹⁸, or —(CR¹⁹R²⁰)ₙ₃OR¹⁷;
R⁵, R⁶, R¹², R¹³, R¹⁴ and R¹⁵ are each independently selected from one or more of H, C₁₋₆alkyl-, C₃₋₈cycloalkyl-C₀₋₆alkyl-,C₃₋₈heterocycloalkyl-C₀₋₆alkyl-, aryl-C₀₋₆alkyl-, or heteroaryl-C₁₋₆alkyl-;
R¹⁷, R¹⁸, R¹⁹, and R²⁰ are each independently selected from H, C₁₋₆alkyl-, C₃₋₈cycloalkyl-C₀₋₆alkyl-, C₃₋₈heterocycloalkyl-C₀₋₆alkyl-, aryl-C₀₋₆alkyl-, or heteroaryl-C₁₋₆alkyl-;
—NR⁵R⁶ and —NR¹²R¹³ are each independently a linear structure;
—CR¹⁴R¹⁵ is a linear structure;
—CR¹⁹R²⁰ is a linear structure; and
—NR¹⁷R¹⁸ is a linear structure;
wherein m1, n1 and n3 are each independently selected from 0, 1 or 2.

In some embodiments, the compound of Formula I is wherein:
X¹ is selected from C₁₋₂ alkyl and C═O;
X² is selected from NR³ and O;
X³ is independently selected from one or more of C₁₋₂ alkyl or C═O;
m and n are each 1;
R¹ is selected from C₀₋₁₂alkyl-, C₃₋₁₂cycloalkyl-C₀₋₁₂alkyl-, aryl-C₀₋₁₂alkyl-, or heteroaryl-C₀₋₁₂alkyl-, any of which is optionally substituted with one or more independent G¹ substituents selected from H, halo, —CN, —CF₃, —OCF₃, —OCHF₂, —C₀₋₁₂alkyl, C₃₋₁₂cycloalkyl-C₀₋₁₂alkyl-, or —OC₀₋₁₂alkyl;
R² is selected from C₀₋₁₂alkyl-, C₃₋₁₂cycloalkyl-C₀₋₁₂alkyl-, or C₃₋₁₂heterocycloalkyl-C₀₋₁₂alkyl-, any of which is optionally substituted with one or more independent G² substituents selected from H or —OC₀₋₁₂alkyl;
R^{2a} is selected from C₀₋₁₂alkyl-, or C₃₋₁₂heterocycloalkyl-C₀₋₁₂alkyl-;
R² and R^{2a} are each independently a linear structure, or, R² and R^{2a} are taken together with the carbon atom to which they are attached to form a 3-12 membered saturated or unsaturated ring, wherein said ring optionally includes one or more additional heteroatoms selected from O, N, or S(O)ₘ₁;
R³ is selected from C₀₋₁₂alkyl-, or C₃₋₁₂cycloalkyl-C₀₋₁₂alkyl-, optionally substituted with one or more independent G³ substituents selected from H, —CN, —NR⁵R⁶, C₃₋₁₂cycloalkyl-C₀₋₁₂alkyl-, —S(O)ₙ₁R¹², or —C(O)OR¹²;
R⁴ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, heteroaryl-C₀₋₁₂alkyl-, or pyridine-N-oxide, any of which is optionally substituted with one or more independent G⁴ substituents selected from H, D, halo, —CN, —OCD₃, -oxo-, —CF₃, —OCF₃, —NR⁵R⁶, —B(OH)₂, —C₀₋₁₂alkyl, aryl-C₀₋₁₂alkyl-, heteroaryl-C₀₋₁₂alkyl-, —OC₀₋₁₂alkyl, —C(O)R¹², —S(O)ₙ₁R¹², —C(O)NR¹²R¹³, —C(O)OR¹², —NR¹²C(O)R¹³, —NR¹²C(O)OR¹³, or —NR¹²S(O)₂R¹³, wherein —OC₀₋₁₂alkyl is optionally substituted with one or more independent Q¹ substituents selected from —CN, NR¹⁷R¹⁸, C₀₋₁₂alkyl-, C₃₋₁₂heterocycloalkyl-C₀₋₁₂alkyl-, —OC₀₋₁₂alkyl, —(CR¹⁹R²⁰)ₙ₃C(O)OR¹⁷, or —(CR¹⁹R²⁰)ₙ₃C(O)NR¹⁷R¹⁸;
R⁵, R⁶, R¹², and R¹³ are each independently selected from one or more of H, C₁₋₆alkyl-, C₃₋₈cycloalkyl-C₀₋₆alkyl-, or C₃₋₈heterocycloalkyl-C₀₋₆alkyl-;
R¹⁷, R¹⁸, R¹⁹, and R²⁰ are each independently selected from H, or C₁₋₆alkyl-;
—NR⁵R⁶ and —NR¹²R¹³ are each independently a linear structure;
—CR¹⁴R¹⁵ is a linear structure;
—CR¹⁹R²⁰ is a linear structure; and
—NR¹⁷R¹⁸ is a linear structure;
wherein m1, n1 and n3 are each independently selected from 0, 1 or 2.

In some embodiments, the compound is of Formula Ia:

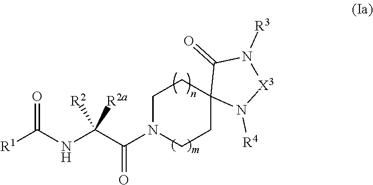

or a pharmaceutically acceptable salt thereof.

In some embodiments, the compound is of Formula Id:

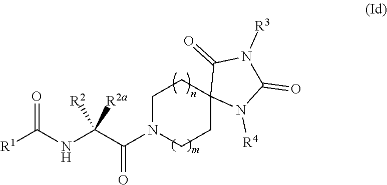

or a pharmaceutically acceptable salt thereof.

In some embodiments, the compound is of Formula Ih:

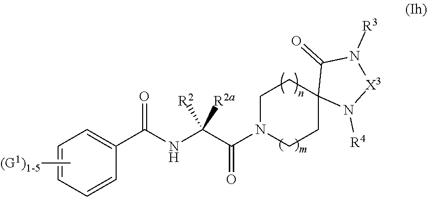

or a pharmaceutically acceptable salt thereof.

In some embodiments, the compound is of Formula Ik:

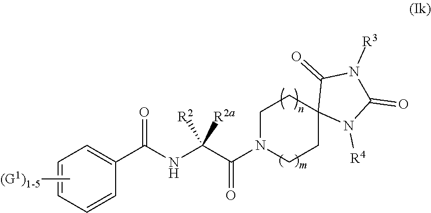

or a pharmaceutically acceptable salt thereof.

In some embodiments, the compound is of Formula Io:

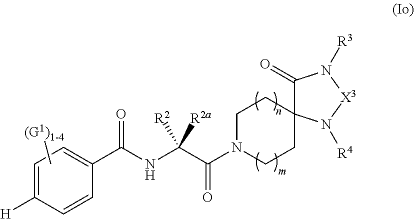

or a pharmaceutically acceptable salt thereof.

In some embodiments, the compound is of Formula Ip:

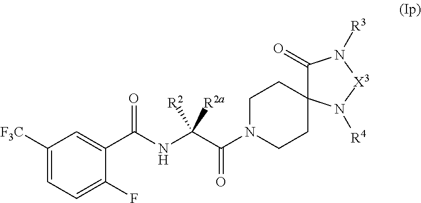

or a pharmaceutically acceptable salt thereof.

In some embodiments, the compound is of the Formula Iq:

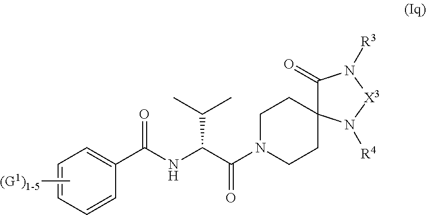

or a pharmaceutically acceptable salt thereof.

In some embodiments, the compounds is of the Formula Ir:

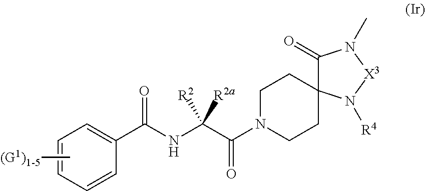

or a pharmaceutically acceptable salt thereof.

In some embodiments, the compound is of the Formula Is:

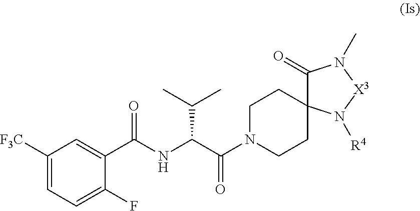

or a pharmaceutically acceptable salt thereof.

In some embodiments, the compound is of the Formula It:

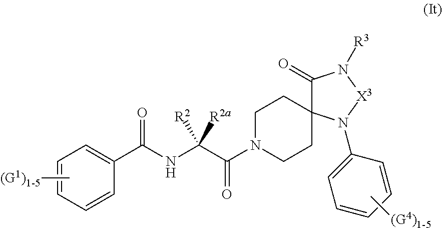

or a pharmaceutically acceptable salt thereof.

In some embodiments, the compound is of the Formula Iu:

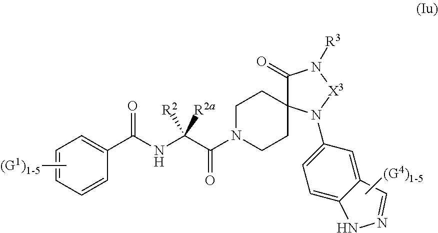

or a pharmaceutically acceptable salt thereof.

In some embodiments, the compound is of the Formula Iv:

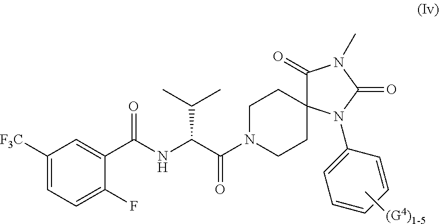

or a pharmaceutically acceptable salt thereof.

In some embodiments, the compound is of the Formula Iw:

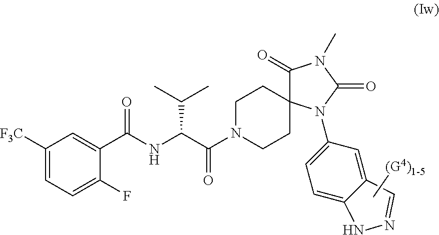

or a pharmaceutically acceptable salt thereof.

In some embodiments, the compound of Formula I is wherein:
R¹ is selected from t-Bu, cyclohexane, adamantyl, phenyl, pyridine or thiazole, each optionally substituted with one or more independent G¹ substituents selected from H, F, Cl, —CN, —CF₃, —OCF₃, —OCH₂F, methyl, ethyl, cyclopropyl, or methoxy;
R² is selected from H, methyl, ethyl, isopropyl, sec-butyl, cyclopropyl, cyclobutyl, cyclopentyl, cyclohexyl, or tetrahydro-2H-pyran, wherein the ethyl group is optionally substitued with one or more G¹ substituent selected from —OMe;
R^{2a} is selected from H or tetrahydro-2H-pyran;
or R² and R^{2a} are taken together with the carbon atom to which they are attached to form a cyclopropane or oxetane;
R³ is methyl, ethyl, propyl, or cyclopropylmethyl, optionally substituted with one or more independent G³ substituents selected from —CN, —NMe₂, cyclopropyl, —SO₂Me, or —COOH; and
each G⁴ substituent is selected from H, D, F, Cl, Br, —CN, —OCD₃, oxo, —CF₃, —OCF₃, —NH(azetidine), —NH(oxetane), —B(OH)₂, Me, triazole, tetrazole, —OMe, -OEt, —SO₂Me, —C(O)NH₂, —COOH, —C(O)OMe, —NHC(O)-cyclopropane, —NHC(O)OMe, or —NHSO₂Me, wherein the —OMe and -OEt groups are optionally substituted with one or more independent Q¹ substituents selected from —CN, NMe₂, Me, azetidine, oxetane, —OH, —COOH, or —C(O)NH₂.

In some embodiments, the compound of Formula I has the structure selected from the group consisting of:
(R)—N-(1-Cyclohexyl-2-(3-methyl-2,4-dioxo-1-phenyl-1,3,8-triazaspiro[4.5]decan-8-yl)-2-oxoethyl)-3-methylbenzamide;
(R)-3-Fluoro-N-(3-methyl-1-(3-methyl-2,4-dioxo-1-phenyl-1,3,8-triazaspiro[4.5]decan-8-yl)-1-oxobutan-2-yl)benzamide;
(R)-2-Fluoro-3-methyl-N-(3-methyl-1-(3-methyl-2,4-dioxo-1-phenyl-1,3,8-triazaspiro[4.5]decan-8-yl)-1-oxobutan-2-yl)benzamide;
2-Fluoro-3-methyl-N-((2R,3R)-3-methyl-1-(3-methyl-2,4-dioxo-1-phenyl-1,3,8-triazaspiro[4.5]decan-8-yl)-1-oxopentan-2-yl)benzamide;
(R)—N-(1-(3-(Cyanomethyl)-1-(4-methoxyphenyl)-2,4-dioxo-1,3,8-triazaspiro[4.5]decan-8-yl)-3-methyl-1-oxobutan-2-yl)-5-ethyl-2-fluorobenzamide;
(R)-2-Fluoro-N-(1-(1-(3-fluoro-4-methoxyphenyl)-3-methyl-2,4-dioxo-1,3,8-triazaspiro[4.5]decan-8-yl)-3-methyl-1-oxobutan-2-yl)-5-(trifluoromethyl)benzamide;
(R)—N-(1-(1-(4-Cyanophenyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1-(3-Cyanophenyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(3-methyl-1-(3-methyl-1-(4-(methylsulfonyl)phenyl)-2,4-dioxo-1,3,8-triazaspiro[4.5]decan-8-yl)-1-oxobutan-2-yl)-5-(trifluoromethyl)benzamide;
(R)-2-Fluoro-N-(1-(1-(3-methoxyphenyl)-3-methyl-2,4-dioxo-1,3,8-triazaspiro[4.5]decan-8-yl)-3-methyl-1-oxobutan-2-yl)-5-(trifluoromethyl)benzamide;
(R)-2-Fluoro-N-(3-methyl-1-(3-methyl-1-(3-methyl-2-oxo-2,3-dihydrobenzo[d]oxazol-5-yl)-2,4-dioxo-1,3,8-triazaspiro[4.5]decan-8-yl)-1-oxobutan-2-yl)-5-(trifluoromethyl)benzamide;
(R)-2-Fluoro-N-(3-methyl-1-(3-methyl-1-(1-methyl-1H-indazol-5-yl)-2,4-dioxo-1,3,8-triazaspiro[4.5]decan-8-yl)-1-oxobutan-2-yl)-5-(trifluoromethyl)benzamide;
(R)-2-Fluoro-N-(3-methyl-1-(3-methyl-1-(2-methyl-2H-indazol-5-yl)-2,4-dioxo-1,3,8-triazaspiro[4.5]decan-8-yl)-1-oxobutan-2-yl)-5-(trifluoromethyl)benzamide;
(R)-2-Fluoro-N-(1-(1-(imidazo[1,2-a]pyridin-6-yl)-3-methyl-2,4-dioxo-1,3,8-triazaspiro[4.5]decan-8-yl)-3-methyl-1-oxobutan-2-yl)-5-(trifluoromethyl)benzamide trifluoroacetic acid salt;
(R)-2-Fluoro-N-(3-methyl-1-(3-methyl-1-(3-(methylsulfonyl)phenyl)-2,4-dioxo-1,3,8-triazaspiro[4.5]decan-8-yl)-1-oxobutan-2-yl)-5-(trifluoromethyl)benzamide;
(R)-2-Fluoro-N-(3-methyl-1-(3-methyl-1-(1-methyl-1H-pyrazol-3-yl)-2,4-dioxo-1,3,8-triazaspiro[4.5]decan-8-yl)-1-oxobutan-2-yl)-5-(trifluoromethyl)benzamide;
(R)-2-Fluoro-N-(3-methyl-1-(3-methyl-1-(1-methyl-1H-pyrazol-4-yl)-2,4-dioxo-1,3,8-triazaspiro[4.5]decan-8-yl)-1-oxobutan-2-yl)-5-(trifluoromethyl)benzamide;
(R)-5-Ethyl-2-fluoro-N-(3-methyl-1-(3-methyl-1-(3-methylimidazo[1,5-a]pyridin-6-yl)-2,4-dioxo-1,3,8-triazaspiro[4.5]decan-8-yl)-1-oxobutan-2-yl)benzamide;
(R)-2-Fluoro-N-(3-methyl-1-(3-methyl-1-(2-methyl-1H-benzo[d]imidazol-6-yl)-2,4-dioxo-1,3,8-triazaspiro[4.5]decan-8-yl)-1-oxobutan-2-yl)-5-(trifluoromethyl)benzamide trifluoroacetic acid salt;
(R)-2-Fluoro-N-(3-methyl-1-(3-methyl-2,4-dioxo-1-(1H-pyrrolo[2,3-b]pyridin-5-yl)-1,3,8-triazaspiro[4.5]decan-8-yl)-1-oxobutan-2-yl)-5-(trifluoromethyl)benzamide;
(R)—N-(1-(1-(3,4-Dihydro-2H-benzo[b][1,4]oxazin-7-yl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(3-methyl-1-(3-methyl-2,4-dioxo-1-(6-oxo-1,6-dihydropyridin-3-yl)-1,3,8-triazaspiro[4.5]decan-8-yl)-1-oxobutan-2-yl)-5-(trifluoromethyl)benzamide;
(R)-2-Fluoro-N-(3-methyl-1-(3-methyl-1-(1-methyl-6-oxo-1,6-dihydropyridin-3-yl)-2,4-dioxo-1,3,8-triazaspiro[4.5]decan-8-yl)-1-oxobutan-2-yl)-5-(trifluoromethyl)benzamide;
(R)—N-(1-Cyclopentyl-2-(3-methyl-1-(1-methyl-6-oxo-1,6-dihydropyridin-3-yl)-2,4-dioxo-1,3,8-triazaspiro[4.5]decan-8-yl)-2-oxoethyl)-2-fluoro-5-(trifluoromethyl)benzamide;
(R)-2-Fluoro-N-(1-(1-(6-methoxypyridin-3-yl)-3-methyl-2,4-dioxo-1,3,8-triazaspiro[4.5]decan-8-yl)-3-methyl-1-oxobutan-2-yl)-5-(trifluoromethyl)benzamide;
(R)-2-Fluoro-N-(3-methyl-1-(3-methyl-2,4-dioxo-1-(4-(trifluoromethoxy)phenyl)-1,3,8-triazaspiro[4.5]decan-8-yl)-1-oxobutan-2-yl)-5-(trifluoromethyl)benzamide;
(R)-2-Fluoro-N-(3-methyl-1-(3-methyl-2,4-dioxo-1-(5-benzofuran)-1,3,8-triazaspiro[4.5]decan-8-yl)-1-oxobutan-2-yl)-5-(trifluoromethyl)benzamide;
(R)-2-Fluoro-N-(3-methyl-1-(3-methyl-2,4-dioxo-1-(2-oxo-2,3-dihydrobenzo[d]oxozol-6-yl)-1,3,8-triazaspiro[4.5]decan-8-yl)-1-oxobutan-2-yl)-5-(trifluoromethyl)benzamide;
(R)-2-Fluoro-N-(3-methyl-1-(3-methyl-2,4-dioxo-1-(2-oxo-2,3-dihydrobenzo[d]oxozol-5-yl)-1,3,8-triazaspiro[4.5]decan-8-yl)-1-oxobutan-2-yl)-5-(trifluoromethyl)benzamide;
(R)-2-Fluoro-N-(3-methyl-1-(3-methyl-2,4-dioxo-1-(3-oxo-3,4-dihydro-2H-benzo[b][1,4]oxazin-7-yl)-1,3,8-triazaspiro[4.5]decan-8-yl)-1-oxobutan-2-yl)-5-(trifluoromethyl)benzamide;
(R)-5-Ethyl-2-fluoro-N-(3-methyl-1-(3-methyl-2,4-dioxo-1-(2-oxo-2,3-dihydrobenzo[d]oxozol-6-yl)-1,3,8-triazaspiro[4.5]decan-8-yl)-1-oxobutan-2-yl)benzamide;
(R)—N-(1-(1-(1H-Benzo[d][1,2,3]triazol-5-yl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(3-Methyl-1-(3-methyl-2,4-dioxo-1-phenyl-1,3,8-triazaspiro[4.5]decan-8-yl)-1-oxobutan-2-yl)-3-(trifluoromethyl)benzamide;
(R)-3-Ethyl-2-fluoro-N-(3-methyl-1-(3-methyl-2,4-dioxo-1-phenyl-1,3,8-triazaspiro[4.5]decan-8-yl)-1-oxobutan-2-yl)benzamide;
(R)-3,4-Dichloro-N-(3-methyl-1-(3-methyl-2,4-dioxo-1-phenyl-1,3,8-triazaspiro[4.5]decan-8-yl)-1-oxobutan-2-yl)benzamide;
(R)-2,5-Dichloro-N-(3-methyl-1-(3-methyl-2,4-dioxo-1-phenyl-1,3,8-triazaspiro[4.5]decan-8-yl)-1-oxobutan-2-yl)benzamide;
5-Ethyl-2-fluoro-N-((2R,3S)-3-methoxy-1-(3-methyl-2,4-dioxo-1-phenyl-1,3,8-triazaspiro[4.5]decan-8-yl)-1-oxobutan-2-yl)benzamide;
3-Ethyl-5-fluoro-N-((2R,3S)-3-methoxy-1-(3-methyl-2,4-dioxo-1-phenyl-1,3,8-triazaspiro[4.5]decan-8-yl)-1-oxobutan-2-yl)benzamide;
(R)-5-Ethyl-2-fluoro-N-(3-methyl-1-(3-methyl-2,4-dioxo-1-phenyl-1,3,8-triazaspiro[4.5]decan-8-yl)-1-oxobutan-2-yl)benzamide;
5-Ethyl-2-fluoro-N-((2R,3R)-3-methoxy-1-(1-(4-methyoxyphenyl)-3-methyl-2,4-dioxo-1,3,8-triazaspiro[4.5]decan-8-yl)-1-oxobutan-2-yl)benzamide;
3-fluoro-N-((2R,3R)-1-(1-(4-methoxyphenyl)-3-methyl-2,4-dioxo-1,3,8-triazaspiro[4.5]decan-8-yl)-3-methyl-1-oxobutan-2-yl)-5-methylbenzamide;
(R)-5-Cyclopropyl-2-fluoro-N-(3-methyl-1-(3-methyl-2,4-dioxo-1-phenyl-1,3,8-triazaspiro[4.5]decan-8-yl)-1-oxobutan-2-yl)benzamide;
(R)-5-Chloro-2-fluoro-N-(3-methyl-1-(3-methyl-2,4-dioxo-1-phenyl-1,3,8-triazaspiro[4.5]decan-8-yl)-1-oxobutan-2-yl)benzamide;
(R)-2-Fluoro-N-(3-methyl-1-(3-methyl-2,4-dioxo-1-phenyl-1,3,8-triazaspiro[4.5]decan-8-yl)-1-oxobutan-2-yl)-5-(trifluoromethyl)benzamide;
(R)-5-Cyclopropyl-2-fluoro-N-(1-(1-(4-methoxyphenyl)-3-methyl-2,4-dioxo-1,3,8-triazaspiro[4.5]decan-8-yl)-3-methyl-1-oxobutan-2-yl)benzamide;
(R)-3-Ethyl-N-(3-methyl-1-(3-methyl-2,4-dioxo-1-phenyl-1,3,8-triazaspiro[4.5]decan-8-yl)-1-oxobutan-2-yl)benzamide;
(R)-3-Ethyl-5-fluoro-N-(3-methyl-1-(3-methyl-2,4-dioxo-1-phenyl-1,3,8-triazaspiro[4.5]decan-8-yl)-1-oxobutan-2-yl)benzamide;
(R)-3,5-Dichloro-N-(3-methyl-1-(3-methyl-2,4-dioxo-1-phenyl-1,3,8-triazaspiro[4.5]decan-8-yl)-1-oxobutan-2-yl)benzamide;
5-Cyclopropyl-2-fluoro-N-((2R,3R)-3-methoxy-1-(1-(4-methyoxyphenyl)-3-methyl-2,4-dioxo-1,3,8-triazaspiro[4.5]decan-8-yl)-1-oxobutan-2-yl)benzamide;
(R)-5-Ethyl-2-fluoro-N-(1-(1-(4-methoxyphenyl)-3-methyl-2,4-dioxo-1,3,8-triazaspiro[4.5]decan-8-yl)-3-methyl-1-oxobutan-2-yl)benzamide;
(R)-2-Fluoro-N-(1-(1-(4-methoxyphenyl)-3-methyl-2,4-dioxo-1,3,8-triazaspiro[4.5]decan-8-yl)-3-methyl-1-oxobutan-2-yl)-5-(trifluoromethyl)benzamide;
(R)—N-(1-(1-(1H-indazol-5-yl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1-(1H-Indazol-6-yl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-5-cyclopropyl-2-fluorobenzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-5-ethyl-2-fluorobenzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-2-fluoro-5-methylbenzamide;
(R)—N-(2-(1-(1H-Indazol-5-yl)-3-methyl-2,4-dioxo-1,3,8-triazaspiro[4.5]decan-8-yl)-1-cyclopentyl-2-oxoethyl)-2-fluoro-5-(trifluoromethyl)benzamide;
(R)—N-(2-(1-(1H-Indazol-5-yl)-3-methyl-2,4-dioxo-1,3,8-triazaspiro[4.5]decan-8-yl)-1-cyclobutyl-2-oxoethyl)-2-fluoro-5-(trifluoromethyl)benzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-3-(trifluoromethyl)benzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-3-chloro-5-(trifluoromethyl)benzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-2-methyl-5-(trifluoromethyl)benzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-2-fluoro-5-(trifluoromethoxy)benzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-2-fluoro-5-methoxybenzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-5-(difluoromethoxy)-2-fluorobenzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-2,5-dichlorobenzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-2,5-difluorobenzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-1-admantanecarboxly amide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-2-chloro-5-(trifluoromethyl)benzamide;
(S)—N-(1-(1-(1H-Indazol-5-yl)-3-methyl-2,4-dioxo-1,3,8-triazaspiro[4.5]decan-8-yl)-3-methyl-1-oxobutan-2-yl)-2-fluoro-5-(trifluoromethyl)benzamide;
N-(2-(1-(1H-Indazol-5-yl)-3-methyl-2,4-dioxo-1,3,8-triazaspiro[4.5]decan-8-yl)-2-oxoethyl)-2-fluoro-5-(trifluoromethyl)benzamide;
N-(1-(1-(1H-Indazol-5-yl)-3-methyl-2,4-dioxo-1,3,8-triazaspiro[4.5]decane-8-carbonyl)cyclopropyl)-2-fluoro-5-(trifluoromethyl)benzamide;
(R)—N-(1-(1-(1H-Indazol-5-yl)-3-methyl-2,4-dioxo-1,3,8-triazaspiro[4.5]decan-8-yl)-1-oxopropan-2-yl)-2-fluoro-5-(trifluoromethyl)benzamide;
N-(3-(1-(1H-Indazol-5-yl)-3-methyl-2,4-dioxo-1,3,8-triazaspiro[4.5]decane-8-carbonyl)oxetan-3-yl)-2-fluoro-5-(trifluoromethyl)benzamide;
N-(1-(1-(1H-Indazol-5-yl)-3-methyl-2,4-dioxo-1,3,8-triazaspiro[4.5]decan-8-yl)-2-methyl-1-oxopropan-2-yl)-2-fluoro-5-(trifluoromethyl)benzamide;
(R)—N-(1-(1-(Cyclopropylmethyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1-Cyclopropyl-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(3-methyl-1-(3-methyl-2,4-dioxo-1-(tetrahydro-2H-pyran-4-yl)-1,3,8-triazaspiro[4.5]decan-8-yl)-1-oxobutan-2-yl)-5-(trifluoromethyl)benzamide;
2-Fluoro-N—((R)-3-methyl-1-(3-methyl-2,4-dioxo-1-((S)-1-phenylethyl)-1,3,8-triazaspiro[4.5]decan-8-yl)-1-oxobutan-2-yl)-5-(trifluoromethyl)benzamide;
(R)-2-Fluoro-N-(3-methyl-1-(3-methyl-2,4-dioxo-1-((tetrahydro-2H-pyran-4-yl)methyl)-1,3,8-triazaspiro[4.5]decan-8-yl)-1-oxobutan-2-yl)-5-(trifluoromethyl)benzamide;
2-Fluoro-N—((R)-3-methyl-1-(3-methyl-2,4-dioxo-1-((R)-1-phenylethyl)-1,3,8-triazaspiro[4.5]decan-8-yl)-1-oxobutan-2-yl)-5-(trifluoromethyl)benzamide;
(R)—N-(1-(1-(4-(Azetidin-3-ylmethoxy)phenyl)-3-methyl-2,4-dioxo-1,3,8-triazaspiro[4.5]decan-8-yl)-3-methyl-1-oxobutan-2-yl)-2-fluoro-5-(trifluoromethyl)benzamide trifluoroacetic acid salt;
(R)—N-(1-(1-(4-(cyanomethoxy)phenyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(3-methyl-1-(3-methyl-1-(4-(oxetan-3-ylmethoxy)phenyl)-2,4-dioxo-1,3,8-triazaspiro[4.5]decan-8-yl)-1-oxobutan-2-yl)-5-(trifluoromethyl)benzamide;
(R)-2-Fluoro-N-(1-(1-(4-(2-hydroxyethoxy)phenyl)-3-methyl-2,4-dioxo-1,3,8-triazaspiro[4.5]decan-8-yl)-3-methyl-1-oxobutan-2-yl)-5-(trifluoromethyl)benzamide;
(R)—N-(1-(1-(4-(2-(Dimethylamino)ethoxy)phenyl)-3-methyl-2,4-dioxo-1,3,8-triazaspiro[4.5]decan-8-yl)-3-methyl-1-oxobutan-2-yl)-2-fluoro-5-(trifluoromethyl)benzamide trifluoroacetic acid salt;
(R)—N-(1-(1-(4-(Azetidin-3-ylamino)phenyl)-3-methyl-2,4-dioxo-1,3,8-triazaspiro[4.5]decan-8-yl)-3-methyl-1-oxobutan-2-yl)-2-fluoro-5-(trifluoromethyl)benzamide trifluoroacetic acid salt;
(R)-2-Fluoro-N-(3-methyl-1-(3-methyl-1-(4-(oxetan-3-ylamino)phenyl)-2,4-dioxo-1,3,8-triazaspiro[4.5]decan-8-yl)-1-oxobutan-2-yl)-5-(trifluoromethyl)benzamide;
(R)—N-(1-(1-(4-Cyanobenzyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1-Benzyl-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(1-(1-(4-fluorobenzyl)-3-methyl-2,4-dioxo-1,3,8-triazaspiro[4.5]decan-8-yl)-3-methyl-1-oxobutan-2-yl)-5-(trifluoromethyl)benzamide;
(R)-2-Fluoro-N-(1-(1-(4-methoxybenzyl)-3-methyl-2,4-dioxo-1,3,8-triazaspiro[4.5]decan-8-yl)-3-methyl-1-oxobutan-2-yl)-5-(trifluoromethyl)benzamide;
2-Fluoro-N-((2R)-3-methyl-1-(3-methyl-2,4-dioxo-1-(1-phenylethyl)-1,3,8-triazaspiro[4.5]decan-8-yl)-1-oxobutan-2-yl)-5-(trifluoromethyl)benzamide;
(R)-2-Fluoro-N-(3-methyl-1-(3-methyl-1-(4-(methylsulfonyl)benzyl)-2,4-dioxo-1,3,8-triazaspiro[4.5]decan-8-yl)-1-oxobutan-2-yl)-5-(trifluoromethyl)benzamide;
(R)-2-Fluoro-N-(1-(1-((2-methoxypyridin-4-yl)methyl)-3-methyl-2,4-dioxo-1,3,8-triazaspiro[4.5]decan-8-yl)-3-methyl-1-oxobutan-2-yl)-5-(trifluoromethyl)benzamide;
(R)-2-Fluoro-N-(3-methyl-1-(3-methyl-1-((2-methylpyridin-4-yl)methyl)-2,4-dioxo-1,3,8-triazaspiro[4.5]decan-8-yl)-1-oxobutan-2-yl)-5-(trifluoromethyl)benzamide;
(R)—N-(1-(1-((2,3-Dihydrobenzo[b][1,4]dioxin-6-yl)methyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(1-(1-((6-methoxypyridin-3-yl)methyl)-3-methyl-2,4-dioxo-1,3,8-triazaspiro[4.5]decan-8-yl)-3-methyl-1-oxobutan-2-yl)-5-(trifluoromethyl)benzamide;
(R)—N-(1-(1-((1H-Indazol-5-yl)methyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1-(4-Chlorobenzyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(3-methyl-1-(3-methyl-2,4-dioxo-1-(quinoxalin-6-ylmethyl)-1,3,8-triazaspiro[4.5]decan-8-yl)-1-oxobutan-2-yl)-5-(trifluoromethyl)benzamide;
(R)—N-(1-(1-(3-Cyanobenzyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(1-(1-(imidazo[1,2-a]pyridin-7-ylmethyl)-3-methyl-2,4-dioxo-1,3,8-triazaspiro[4.5]decan-8-yl)-3-methyl-1-oxobutan-2-yl)-5-(trifluoromethyl)benzamide;
(R)-2-Fluoro-N-(3-methyl-1-(3-methyl-1-(3-(methylsulfonyl)benzyl)-2,4-dioxo-1,3,8-triazaspiro[4.5]decan-8-yl)-1-oxobutan-2-yl)-5-(trifluoromethyl)benzamide;
(R)—N-(1-(1-(3-Chlorobenzyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(3-methyl-1-(3-methyl-1-((3-methyl-2-oxo-2,3-dihydrobenzo[d]oxazol-5-yl)methyl)-2,4-dioxo-1,3,8-triazaspiro[4.5]decan-8-yl)-1-oxobutan-2-yl)-5-(trifluoromethyl)benzamide;
(R)-2-Fluoro-N-(3-methyl-1-(3-methyl-1-((2-methyl-1H-benzo[d]imidazol-6-yl)methyl)-2,4-dioxo-1,3,8-triazaspiro[4.5]decan-8-yl)-1-oxobutan-2-yl)-5-(trifluoromethyl)benzamide;
(R)-2-Fluoro-N-(1-(1-(2-fluoro-5-methoxybenzyl)-3-methyl-2,4-dioxo-1,3,8-triazaspiro[4.5]decan-8-yl)-3-methyl-1-oxobutan-2-yl)-5-(trifluoromethyl)benzamide;
(R)-2-Fluoro-N-(1-(1-(2-fluoro-4-methoxybenzyl)-3-methyl-2,4-dioxo-1,3,8-triazaspiro[4.5]decan-8-yl)-3-methyl-1-oxobutan-2-yl)-5-(trifluoromethyl)benzamide;
(R)—N-(1-Cyclopropyl-2-(3-methyl-2,4-dioxo-1-(quinoxalin-6-ylmethyl)-1,3,8-triazaspiro[4.5]decan-8-yl)-2-oxoethyl)-2-fluoro-5-(trifluoromethyl)benzamide;
(R)—N-(1-Cyclobutyl-2-(3-methyl-2,4-dioxo-1-(quinoxalin-6-ylmethyl)-1,3,8-triazaspiro[4.5]decan-8-yl)-2-oxoethyl)-2-fluoro-5-(trifluoromethyl)benzamide;
(R)-2-Fluoro-N-(3-methyl-1-(3-methyl-2,4-dioxo-1-(pyrimidin-2-ylmethyl)-1,3,8-triazaspiro[4.5]decan-8-yl)-1-oxobutan-2-yl)-5-(trifluoromethyl)benzamide;
(R)—N-(1-Cyclopentyl-2-(3-methyl-2,4-dioxo-1-(quinoxalin-6-ylmethyl)-1,3,8-triazaspiro[4.5]decan-8-yl)-2-oxoethyl)-2-fluoro-5-(trifluoromethyl)benzamide;
(R)—N-(1-Cyclopentyl-2-(3-methyl-1-((2-methyl-[1,2,4]triazolo[1,5-a]pyridin-7-yl)methyl)-2,4-dioxo-1,3,8-triazaspiro[4.5]decan-8-yl)-2-oxoethyl)-2-fluoro-5-(trifluoromethyl)benzamide;
(R)-2-Fluoro-N-(3-methyl-1-(3-methyl-1-((2-methyl-[1,2,4]triazolo[1,5-a]pyridin-7-yl)methyl)-2,4-dioxo-1,3,8-triazaspiro[4.5]decan-8-yl)-1-oxobutan-2-yl)-5-(trifluoromethyl)benzamide;
(R)-2-Fluoro-N-(3-methyl-1-(3-methyl-1-((1-methyl-1H-pyrazol-4-yl)methyl)-2,4-dioxo-1,3,8-triazaspiro[4.5]decan-8-yl)-1-oxobutan-2-yl)-5-(trifluoromethyl)benzamide;
(R)-2-Fluoro-N-(3-methyl-1-(3-methyl-1-((2-methylbenzo[d]oxazol-5-yl)methyl)-2,4-dioxo-1,3,8-triazaspiro[4.5]decan-8-yl)-1-oxobutan-2-yl)-5-(trifluoromethyl)benzamide;
(R)-2-Fluoro-N-(3-methyl-1-(3-methyl-1-((4-methylthiazol-2-yl)methyl)-2,4-dioxo-1,3,8-triazaspiro[4.5]decan-8-yl)-1-oxobutan-2-yl)-5-(trifluoromethyl)benzamide;
(R)-2-Fluoro-N-(3-methyl-1-(3-methyl-1-((2-methylbenzo[d]oxazol-6-yl)methyl)-2,4-dioxo-1,3,8-triazaspiro[4.5]decan-8-yl)-1-oxobutan-2-yl)-5-(trifluoromethyl)benzamide;
(R)—N-(1-(1-(2-Chloro-4-methoxybenzyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(3-methyl-1-(3-methyl-1-((1-methyl-6-oxo-1,6-dihydropyridin-3-yl)methyl)-2,4-dioxo-1,3,8-triazaspiro[4.5]decan-8-yl)-1-oxobutan-2-yl)-5-(trifluoromethyl)benzamide;
(R)—N-(1-(1-((1-Acetylazetidin-3-yl)methyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1,3-Dimethyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1-Ethyl-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(1-(1-isopentyl-3-methyl-2,4-dioxo-1,3,8-triazaspiro[4.5]decan-8-yl)-3-methyl-1-oxobutan-2-yl)-5-(trifluoromethyl)benzamide;
(R)—N-(1-(1-Cyclopentyl-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(3-methyl-1-(3-methyl-2,4-dioxo-1,3,8-triazaspiro[4.5]decan-8-yl)-1-oxobutan-2-yl)-5-(trifluoromethyl)benzamide;
(R)-Methyl-4-(8-(2-(2-fluoro-5-(trifluoromethyl)benzamido)-3-methylbutanoyl)-3-methyl-2,4-dioxo-1,3,8-triazaspiro[4.5]decan-1-yl)phenylcarbamate;
(R)—N-(1-(1-(4-(Cyclopropanecarboxamido)phenyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-3-Methyl-N-(2-(3-methyl-4-oxo-1-phenyl-1,3,8-triazaspiro[4.5]decan-8-yl)-2-oxo-1-(tetrahydro-2H-pyran-4-yl)ethyl)benzamide;
(S)-3-Methyl-N-(2-(3-methyl-4-oxo-1-phenyl-1,3,8-triazaspiro[4.5]decan-8-yl)-2-oxo-1-(tetrahydro-2H-pyran-4-yl)ethyl)benzamide;
(R)—N-(1-Cyclohexyl-2-(3-methyl-4-oxo-1-phenyl-1,3,8-triazaspiro[4.5]decan-8-yl)-2-oxoethyl)-6-methylpicolinamide trifluoroacetic acid salt;
(R)—N,3-Dimethyl-N-(3-methyl-1-(3-methyl-4-oxo-1-phenyl-1,3,8-triazaspiro[4.5]decan-8-yl)-1-oxobutan-2-yl)benzamide;
5-Ethyl-2-fluoro-N-((2R,3S)-3-methoxy-1-(3-methyl-4-oxo-1-phenyl-1,3,8-triazaspiro[4.5]decane-8-yl)-1-oxobutan-2-yl)benzamide;
N-((2R,3S)-3-Methoxy-1-(3-methyl-4-oxo-1-phenyl-1,3,8-triazaspiro[4.5]decan-8-yl)-1-oxobutan-2-yl)-3-methylbenzamide;
3-Ethyl-N-((2R,3S)-3-methoxy-1-(3-methyl-4-oxo-1-phenyl-1,3,8-triazaspiro[4.5]decan-8-yl)-1-oxobutan-2-yl)benzamide;
3-Ethyl-5-fluoro-N-((2R,3S)-3-methoxy-1-(3-methyl-4-oxo-1-phenyl-1,3,8-triazaspiro[4.5]decan-8-yl)-1-oxobutan-2-yl)benzamide;
(R)-2-Methyl-N-(3-methyl-1-(3-methyl-4-oxo-1-phenyl-1,3,8-triazaspiro[4.5]decan-8-yl)-1-oxobutan-2-yl)isonicotinamide;
(R)-3-Ethyl-N-(3-methyl-1-(3-methyl-4-oxo-1-phenyl-1,3,8-triazaspiro[4.5]decan-8-yl)-1-oxobutan-2-yl)benzamide;
(R)-4-Fluoro-3-methyl-N-(3-methyl-1-(3-methyl-4-oxo-1-phenyl-1,3,8-triazaspiro[4.5]decan-8-yl)-1-oxobutan-2-yl)benzamide;
(R)-3-Fluoro-5-methyl-N-(3-methyl-1-(3-methyl-4-oxo-1-phenyl-1,3,8-triazaspiro[4.5]decan-8-yl)-1-oxobutan-2-yl)benzamide;
(R)-2-Fluoro-5-methyl-N-(3-methyl-1-(3-methyl-4-oxo-1-phenyl-1,3,8-triazaspiro[4.5]decan-8-yl)-1-oxobutan-2-yl)benzamide;
(R)-3-Cyclopropyl-N-(3-methyl-1-(3-methyl-4-oxo-1-phenyl-1,3,8-triazaspiro[4.5]decan-8-yl)-1-oxobutan-2-yl)benzamide;
(R)-3-Chloro-4-cyano-N-(3-methyl-1-(3-methyl-4-oxo-1-phenyl-1,3,8-triazaspiro[4.5]decan-8-yl)-1-oxobutan-2-yl)benzamide;
(R)-5-Ethyl-2-fluoro-N-(3-methyl-1-(3-methyl-4-oxo-1-phenyl-1,3,8-triazaspiro[4.5]decan-8-yl)-1-oxobutan-2-yl)benzamide;
(R)—N-(1-(1-(4-Carbamoylphenyl)-3-methyl-4-oxo-1,3,8-triazaspiro[4.5]decan-8-yl)-3-methyl-1-oxobutan-2-yl)-2-fluoro-3-methylbenzamide;
(R)—N-(1-(1-(3-Carbamoylphenyl)-3-methyl-4-oxo-1,3,8-triazaspiro[4.5]decan-8-yl)-3-methyl-1-oxobutan-2-yl)-2-fluoro-3-methylbenzamide;
(R)-4-(8-(2-(2-Fluoro-3-methylbenzamido)-3-methylbutanoyl)-3-methyl-4-oxo-1,3,8-triazaspiro[4.5]decan-1-yl)phenylboronic acid;
(R)-3-(8-(2-(2-Fluoro-3-methylbenzamido)-3-methylbutanoyl)-3-methyl-4-oxo-1,3,8-triazaspiro[4.5]decan-1-yl)phenylboronic acid;
(R)-4-(8-(2-(2-Fluoro-5-(trifluoromethyl)benzamido)-3-methylbutanoyl)-3-methyl-4-oxo-1,3,8-triazaspiro[4.5]decan-1-yl)benzoic acid;
(R)-4-(8-(2-(2-Fluoro-3-methylbenzamido)-3-methylbutanoyl)-3-methyl-4-oxo-1,3,8-triazaspiro[4.5]decan-1-yl)benzoic acid;
(R)-4-(8-(2-(2-Fluoro-5-(trifluoromethyl)benzamido)-3-methylbutanoyl)-3-methyl-4-oxo-1,3,8-triazaspiro[4.5]decan-1-yl)-2-methoxybenzoic acid;
(R)-4-(8-(2-(5-Ethyl-2-fluorobenzamido)-3-methylbutanoyl)-3-methyl-4-oxo-1,3,8-triazaspiro[4.5]decan-1-yl)benzoic acid;
(R)-4-(8-(2-(5-Cyclopropyl-2-fluorobenzamido)-3-methylbutanoyl)-3-methyl-4-oxo-1,3,8-triazaspiro[4.5]decan-1-yl)benzoic acid;
(R)-2-Chloro-4-(8-(2-(2-fluoro-5-(trifluoromethyl)benzamido)-3-methylbutanoyl)-3-methyl-4-oxo-1,3,8-triazaspiro[4.5]decan-1-yl)benzoic acid;
(R)-4-(3-(Cyclopropylmethyl)-8-(2-(2-fluoro-5-(trifluoromethyl)benzamido)-3-methylbutanoyl)-4-oxo-1,3,8-triazaspiro[4.5]decan-1-yl)benzoic acid;
(R)-4-(8-(2-Cyclopentyl-2-(2-fluoro-5-(trifluoromethyl)benzamido)acetyl)-3-methyl-4-oxo-1,3,8-triazaspiro[4.5]decan-1-yl)benzoic acid;
(R)—N-(1-(1-(4-Carbamoylphenyl)-3-methyl-4-oxo-1,3,8-triazaspiro[4.5]decan-8-yl)-3-methyl-1-oxobutan-2-yl)-2-fluoro-5-(trifluoromethyl)benzamide;
(R)-4-(8-(2-(2-Fluoro-5-(trifluoromethyl)benzamido)-3-methylbutanoyl)-3-methyl-4-oxo-1,3,8-triazaspiro[4.5]decan-1-yl)-2-methylbenzoic acid;
(R)-4-(8-(2-(2-Fluoro-5-(trifluoromethyl)benzamido)-3,3-dimethylbutanoyl)-3-methyl-4-oxo-1,3,8-triazaspiro[4.5]decan-1-yl)benzoic acid;
(R)-4-(8-(2-(3-Chloro-5-(trifluoromethyl)benzamido)-3-methylbutanoyl)-3-methyl-4-oxo-1,3,8-triazaspiro[4.5]decan-1-yl)benzoic acid;
(R)-4-(3-Methyl-8-(3-methyl-2-(2-methyl-5-(trifluoromethyl)benzamido)butanoyl)-4-oxo-1,3,8-triazaspiro[4.5]decan-1-yl)benzoic acid;
(R)-4-(8-(2-(2-Fluoro-5-(trifluoromethoxy)benzamido)-3-methylbutanoyl)-3-methyl-4-oxo-1,3,8-triazaspiro[4.5]decan-1-yl)benzoic acid;
(R)-4-(8-(2-(2-Fluoro-5-methoxybenzamido)-3-methylbutanoyl)-3-methyl-4-oxo-1,3,8-triazaspiro[4.5]decan-1-yl)benzoic acid;
(R)-4-(8-(2-(1-Admantanecarboxamido)-3-methylbutanoyl)-3-methyl-4-oxo-1,3,8-triazaspiro[4.5]decan-1-yl)benzoic acid;
(R)-4-(8-(2-(5-(Difluoromethoxy)-2-fluorobenzamido)-3-methylbutanoyl)-3-methyl-4-oxo-1,3,8-triazaspiro[4.5]decan-1-yl)benzoic acid;
(R)-4-(8-(2-(5-(Difluoromethoxy)-2-fluorobenzamido)-3-methylbutanoyl)-3-methyl-2,4-dioxo-1,3,8-triazaspiro[4.5]decan-1-yl)benzoic acid;
(R)-4-(8-(2-(2-Fluoro-5-(trifluoromethoxy)benzamido)-3-methylbutanoyl)-3-methyl-2,4-dioxo-1,3,8-triazaspiro[4.5]decan-1-yl)benzoic acid;
(R)-2-(4-(8-(2-(5-Ethyl-2-fluorobenzamido)-3-methylbutanoyl)-3-methyl-4-oxo-1,3,8-triazaspiro[4.5]decan-1-yl)phenoxy)acetic acid;
(R)—N-(1-(1-(4-(2-Amino-2-oxoethoxy)phenyl)-3-methyl-4-oxo-1,3,8-triazaspiro[4.5]decan-8-yl)-3-methyl-1-oxobutan-2-yl)-5-ethyl-2-fluorobenzamide;
(R)—N-(1-(1-(4-(Cyanomethoxy)phenyl)-3-methyl-4-oxo-1,3,8-triazaspiro[4.5]decan-8-yl)-3-methyl-1-oxobutan-2-yl)-5-ethyl-2-fluorobenzamide;
(R)—N-(1-(1-(4-(2-(Dimethylamino)ethoxy)phenyl)-3-methyl-4-oxo-1,3,8-triazaspiro[4.5]decan-8-yl)-3-methyl-1-oxobutan-2-yl)-2-fluoro-5-(trifluoromethyl)benzamide trifluoroacetic acid salt;
(R)—N-(1-(1-(4-Acetamidophenyl)-3-methyl-4-oxo-1,3,8-triazaspiro[4.5]decan-8-yl)-3-methyl-1-oxobutan-2-yl)-5-ethyl-2-fluorobenzamide;
(R)-5-Ethyl-2-fluoro-N-(3-methyl-1-(3-methyl-1-(4-(methylsulfonamido)phenyl)-4-oxo-1,3,8-triazaspiro[4.5]decan-8-yl)-1-oxobutan-2-yl)benzamide;
(R)-2-Fluoro-N-(3-methyl-1-(3-methyl-1-(4-(methylsulfonamido)phenyl)-4-oxo-1,3,8-triazaspiro[4.5]decan-8-yl)-1-oxobutan-2-yl)-5-(trifluoromethyl)benzamide;
(R)-Methyl-4-(8-(2-(2-fluoro-5-(trifluoromethyl)benzamido)-3-methylbutanoyl)-3-methyl-4-oxo-1,3,8-triazaspiro[4.5]decan-1-yl)phenylcarbamate;
(R)-2-(8-(2-(3-Fluoro-5-methylbenzamido)-3-methylbutanoyl)-4-oxo-1-phenyl-1,3,8-triazaspiro[4.5]decan-3-yl)acetic acid;
(R)-2-Fluoro-3-methyl-N-(3-methyl-1-(3-(3-(methylsulfonyl)propyl)-4-oxo-1-phenyl-1,3,8-triazaspiro[4.5]decan-8-yl)-1-oxobutan-2-yl)benzamide;
(R)—N-(1-(3-(Cyanomethyl)-4-oxo-1-phenyl-1,3,8-triazaspiro[4.5]decan-8-yl)-3-methyl-1-oxobutan-2-yl)-5-ethyl-2-fluorobenzamide;
(R)—N-(1-(1-(4-Cyanophenyl)-3-methyl-4-oxo-1,3,8-triazaspiro[4.5]decan-8-yl)-3-methyl-1-oxobutan-2-yl)-2-fluoro-3-methylbenzamide;
(R)—N-(1-(1-(3-Cyanophenyl)-3-methyl-4-oxo-1,3,8-triazaspiro[4.5]decan-8-yl)-3-methyl-1-oxobutan-2-yl)-2-fluoro-3-methylbenzamide;
(R)—N-(1-(1-(4-Chlorophenyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(1-(1-(4-fluorophenyl)-3-methyl-2,4-dioxo-1,3,8-triazaspiro[4.5]decan-8-yl)-3-methyl-1-oxobutan-2-yl)-5-(trifluoromethyl)benzamide;
(R)—N-(1-(1-(3,4-Dichlorophenyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(3-methyl-1-(3-methyl-2,4-dioxo-1-p-tolyl-1,3,8-triazaspiro[4.5]decan-8-yl)-1-oxobutan-2-yl)-5-(trifluoromethyl)benzamide;
(R)—N-(1-(1-(1H-Indol-5-yl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(3-methyl-1-(3-methyl-2,4-dioxo-1-(4-(trifluoromethyl)phenyl)-1,3,8-triazaspiro[4.5]decan-8-yl)-1-oxobutan-2-yl)-5-(trifluoromethyl)benzamide;
(R)—N-(1-(1-(3-Chlorophenyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(3-methyl-1-(3-methyl-2,4-dioxo-1-(pyridin-2-yl)-1,3,8-triazaspiro[4.5]decan-8-yl)-1-oxobutan-2-yl)-5-(trifluoromethyl)benzamide;
(R)-2-Fluoro-N-(3-methyl-1-(3-methyl-2,4-dioxo-1-(pyridin-3-yl)-1,3,8-triazaspiro[4.5]decan-8-yl)-1-oxobutan-2-yl)-5-(trifluoromethyl)benzamide;
(R)-2-Fluoro-N-(3-methyl-1-(3-methyl-2,4-dioxo-1-(4-deuterium-phenyl-1,3,8-triazaspiro[4.5]decan-8-yl)-1-oxobutan-2-yl)-5-(trifluoromethyl)benzamide;
(R)—N-(1-(1-(Benzo[d][1,3]dioxol-5-yl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1-(3-Chlorophenyl)-3-methyl-4-oxo-1,3,8-triazaspiro[4.5]decan-8-yl)-3-methyl-1-oxobutan-2-yl)-3-methylbenzamide;
(R)—N-(1-(1-(4-Chlorophenyl)-3-methyl-4-oxo-1,3,8-triazaspiro[4.5]decan-8-yl)-3-methyl-1-oxobutan-2-yl)-3-methylbenzamide;
(R)—N-(1-(1-(3-Bromophenyl)-3-methyl-4-oxo-1,3,8-triazaspiro[4.5]decan-8-yl)-3-methyl-1-oxobutan-2-yl)-3-methylbenzamide;
(R)-3-Methyl-N-(3-methyl-1-(3-methyl-4-oxo-1-(pyridin-3-yl)-1,3,8-triazaspiro[4.5]decan-8-yl)-1-oxobutan-2-yl)benzamide;
(R)—N-(1-(1-(2-Chlorophenyl)-3-methyl-4-oxo-1,3,8-triazaspiro[4.5]decan-8-yl)-3-methyl-1-oxobutan-2-yl)-3-methylbenzamide;
(R)—N-(1-(1-(4-Methoxyphenyl)-3-methyl-4-oxo-1,3,8-triazaspiro[4.5]decan-8-yl)-3-methyl-1-oxobutan-2-yl)-3-methylbenzamide;
(R)—N-(1-(1-(3-Methoxyphenyl)-3-methyl-4-oxo-1,3,8-triazaspiro[4.5]decan-8-yl)-3-methyl-1-oxobutan-2-yl)-3-methylbenzamide;
(R)—N-(1-(1-(4-Bromophenyl)-3-methyl-4-oxo-1,3,8-triazaspiro[4.5]decan-8-yl)-3-methyl-1-oxobutan-2-yl)-3-methylbenzamide;
(R)-5-Ethyl-2-fluoro-N-(3-methyl-1-(3-methyl-1-(4-(methylsulfonyl)phenyl)-4-oxo-1,3,8-triazaspiro[4.5]decan-8-yl)-1-oxobutan-2-yl)benzamide;
(R)-5-Ethyl-2-fluoro-N-(3-methyl-1-(3-methyl-1-(1-methyl-6-oxo-1,6-dihydropyridin-3-yl)-4-oxo-1,3,8-triazaspiro[4.5]decan-8-yl)-1-oxobutan-2-yl)benzamide;
(R)-5-Ethyl-2-fluoro-N-(1-(1-(4-methoxyphenyl)-3-methyl-4-oxo-1,3,8-triazaspiro[4.5]decan-8-yl)-3-methyl-1-oxobutan-2-yl)benzamide;
(R)-5-Ethyl-2-fluoro-N-(3-methyl-1-(3-methyl-1-(3-methyl-2-oxo-2,3-dihydrobenzo[d]oxazol-5-yl)-4-oxo-1,3,8-triazaspiro[4.5]decan-8-yl)-1-oxobutan-2-yl)benzamide;
(R)-2-Fluoro-N-(3-methyl-1-(3-methyl-1-(1-methyl-6-oxo-1,6-dihydropyridin-3-yl)-4-oxo-1,3,8-triazaspiro[4.5]decan-8-yl)-1-oxobutan-2-yl)-5-(trifluoromethyl)benzamide;
(R)-2-Fluoro-N-(3-methyl-1-(3-methyl-1-(4-(methylsulfonyl)phenyl)-4-oxo-1,3,8-triazaspiro[4.5]decan-8-yl)-1-oxobutan-2-yl)-5-(trifluoromethyl)benzamide;
(R)-2-Fluoro-N-(1-(1-(imidazo[1,2-a]pyridin-6-yl)-3-methyl-4-oxo-1,3,8-triazaspiro[4.5]decan-8-yl)-3-methyl-1-oxobutan-2-yl)-5-(trifluoromethyl)benzamide trifluoroacetic acid salt;
(R)-3-Chloro-N-(3-methyl-1-(3-methyl-1-(1-methyl-6-oxo-1,6-dihydropyridin-3-yl)-4-oxo-1,3,8-triazaspiro[4.5]decan-8-yl)-1-oxobutan-2-yl)-5-(trifluoromethyl)benzamide;
(R)—N-(1-Cyclopentyl-2-(3-methyl-1-(1-methyl-6-oxo-1,6-dihydropyridin-3-yl)-4-oxo-1,3,8-triazaspiro[4.5]decan-8-yl)-2-oxoethyl)-2-fluoro-5-(trifluoromethyl)benzamide;
2-Fluoro-N-((2R)-1-(1-(4-methoxylpenyl)-2,3-dimethyl-4-oxo-1,3,8-triazaspiro[4.5]decan-8-yl)-3-methyl-1-oxobutan-2-yl)-5-(trifluoromethyl)benzamide;
4-((R)-8-((R)-2-(5-Ethyl-2-fluorobenzamido)-3-methylbutanoyl)-2,3-dimethyl-4-oxo-1,3,8-triazaspiro[4.5]decan-1-yl)benzoic acid;
4-((S)-8-((R)-2-(5-Ethyl-2-fluorobenzamido)-3-methylbutanoyl)-2,3-dimethyl-4-oxo-1,3,8-triazaspiro[4.5]decan-1-yl)benzoic acid;
4-((R)-8-((R)-2-(2-Fluoro-5-(trifluoromethyl)benzamido)-3-methylbutanoyl)-2,3-dimethyl-4-oxo-1,3,8-triazaspiro[4.5]decan-1-yl)benzoic acid;
4-((S)-8-((R)-2-(2-Fluoro-5-(trifluoromethyl)benzamido)-3-methylbutanoyl)-2,3-dimethyl-4-oxo-1,3,8-triazaspiro[4.5]decan-1-yl)benzoic acid;
(R)—N-(1-(1-(1H-Indazol-5-yl)-3-methyl-4-oxo-1,3,8-triazaspiro[4.5]decan-8-yl)-3-methyl-1-oxobutan-2-yl)-2-fluoro-5-(trifluoromethyl)benzamide;
(R)—N-(1-(1-(1H-Indazol-5-yl)-3-methyl-4-oxo-1,3,8-triazaspiro[4.5]decan-8-yl)-3-methyl-1-oxobutan-2-yl)-5-ethyl-2-fluorobenzamide;
(R)—N-(1-(1-(1H-Benzo[d]imidazol-6-yl)-3-methyl-4-oxo-1,3,8-triazaspiro[4.5]decan-8-yl)-3-methyl-1-oxobutan-2-yl)-5-ethyl-2-fluorobenzamide;
(R)-3-Methyl-N-(3-methyl-1-oxo-1-(2-oxo-1-phenyl-3-oxa-1,8-diazaspiro[4.5]decan-8-yl)butan-2-yl)benzamide;
(R)-2-Fluoro-N-(3-methyl-1-(3-methyl-1-(4-(oxetan-3-ylamino)phenyl)-4-oxo-1,3,8-triazaspiro[4.5]decan-8-yl)-1-oxobutan-2-yl)-5-(trifluoromethyl)benzamide;
(R)-2-Fluoro-N-(1-(1-((3-methoxycyclobutyl)methyl)-3-methyl-2,4-dioxo-1,3,8-triazaspiro[4.5]decan-8-yl)-3-methyl-1-oxobutan-2-yl)-5-(trifluoromethyl)benzamide;
(R)—N-(1-(1-(4-(1H-1,2,4-Triazol-3-yl)phenyl)-3-methyl-4-oxo-1,3,8-triazaspiro[4.5]decan-8-yl)-3-methyl-1-oxobutan-2-yl)-2-fluoro-5-(trifluoromethyl)benzamide;
(R)-Methyl-4-(8-(2-(2-fluoro-5-(trifluoromethyl)benzamido)-3-methylbutanoyl)-3-methyl-2,4-dioxo-1,3,8-triazaspiro[4.5]decan-1-yl)benzoate;
(R)-4-(8-(2-(5-Cyclopropyl-2-fluorobenzamido)-3-methylbutanoyl)-3-methyl-2,4-dioxo-1,3,8-triazaspiro[4.5]decan-1-yl)benzoic acid;
(R)-4-(8-(2-(2-Fluoro-5-(trifluoromethyl)benzamido)-3-methylbutanoyl)-3-methyl-2,4-dioxo-1,3,8-triazaspiro[4.5]decan-1-yl)benzoic acid;
(R)—N-(1-(1-(4-(2H-Tetrazol-5-yl)phenyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1-(4-(2H-Tetrazol-5-yl)phenyl)-3-methyl-2,4-dioxo-1,3,8-triazaspiro[4.5]decan-8-yl)-3-methyl-1-oxobutan-2-yl)-2-fluoro-5-(trifluoromethoxy)benzamide;
(R)—N-(1-(1-(4-(2H-Tetrazol-5-yl)phenyl)-3-methyl-2,4-dioxo-1,3,8-triazaspiro[4.5]decan-8-yl)-3-methyl-1-oxobutan-2-yl)-5-(difluoromethoxy)-2-fluorobenzamide;
(R)—N-(1-(1-(4-(2H-Tetrazol-5-yl)phenyl)-3-methyl-4-oxo-1,3,8-triazaspiro[4.5]decan-8-yl)-3-methyl-1-oxobutan-2-yl)-2-fluoro-5-(trifluoromethyl)benzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)picolinamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)nicotinamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)cyclohexanecarboxamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)isonicotinamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)pivalamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)benzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)thiazole-2-carboxamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-4-(trifluoromethyl)thiazole-2-carboxamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-2-fluoro-N-methyl-5-(trifluoromethyl)benzamide;
(R)-4-(3-(2-(Dimethylamino)ethyl)-8-(2-(2-fluoro-5-(trifluoromethyl)benzamido)-3-methylbutanoyl)-2,4-dioxo-1,3,8-triazaspiro[4.5]decan-1-yl)benzoic acid trifluoroacetic acid salt;
(R)-4-(3-(2-(Dimethylamino)ethyl)-8-(2-(2-fluoro-5-(trifluoromethyl) benzamido)-3-methylbutanoyl)-4-oxo-1,3,8-triazaspiro[4.5]decan-1-yl)benzoic acid trifluoroacetic acid salt;
(R)-2-Fluoro-N-(3-methyl-1-(3-methyl-2,4-dioxo-1-(pyridin-4-yl)-1,3,8-triazaspiro[4.5]decan-8-yl)-1-oxobutan-2-yl)-5-(trifluoromethyl)benzamide;
(R)-4-(8-(2-(2-Fluoro-5-(trifluoromethyl)benzamido)-3-methylbutanoyl)-3-methyl-2,4-dioxo-1,3,8-triazaspiro[4.5]decan-1-yl)pyridine 1-oxide;
(R)-2-Fluoro-N-(1-(1-(4-methoxycyclohexyl)-3-methyl-2,4-dioxo-1,3,8-triazaspiro[4.5]decan-8-yl)-3-methyl-1-oxobutan-2-yl)-5-(trifluoromethyl)benzamide;
(R)—N-(1-(1-Cyclohexyl-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(3-Methyl-1-(3-methyl-2,4-dioxo-1-(4-methoxy-d3-phenyl)-1,3,8-triazaspiro[4.5]decan-8-yl)-1-oxobutan-2-yl)-3-(trifluoromethyl)-5-fluorobenzamide;
(R)—N-(3-Methyl-1-(3-methyl-2,4-dioxo-1-(4-ethoxyphenyl)-1,3,8-triazaspiro[4.5]decan-8-yl)-1-oxobutan-2-yl)-3-(trifluoromethyl)-5-fluorobenzamide;
(R)—N-(1-(1-(Benzo[d]oxazol-5-yl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1-(2-Methylbenzo[d]oxazol-5-yl)-3-methyl-2,4-dioxo-1,3,8-triazaspiro[4.5]decan-8-yl)-3-methyl-1-oxobutan-2-yl)-2-fluoro-5-(trifluoromethyl)benzamide;
or a pharmaceutically acceptable salt thereof.

In some embodiments, the compound of Formula I is selected from the group consisting of:
(R)-2-Fluoro-N-(3-methyl-1-(3-methyl-2,4-dioxo-1-(2-oxo-2,3-dihydrobenzo[d]oxazol-6-yl)-1,3,8-triazaspiro[4.5]decan-8-yl)-1-oxobutan-2-yl)-5-(trifluoromethyl)benzamide;
(R)-2-Fluoro-N-(3-methyl-2,4-dioxo-1,3,8-triazaspiro[4.5]decan-8-yl)-3-methyl-1-oxobutan-2-yl)-1-(oxobutan-2-yl)-5-(trifluoromethyl)benzamide;
(R)—N-(1-(1-(4-methoxyphenyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1-(1H-indazol-5-yl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1-(1H-indazol-5-yl)-3-methyl-2,4-dioxo-1,3,8-triazaspiro[4.5]decan-8-yl)-3-methyl-1-oxobutan-2-yl)-3-(trifluoromethyl)benzamide;
(R)—N-(1-(1-(1H-indazol-5-yl)-3-methyl-2,4-dioxo-1,3,8-triazaspiro[4.5]decan-8-yl)-3-methyl-1-oxobutan-2-yl)-2-fluoro-5-(trifluoromethoxy)benzamide;
(R)-4-(8-(2-(2-Fluoro-5-(trifluoromethyl)benzamido)-3-methylbutanoyl)-3-methyl-4-oxo-1,3,8-triazaspiro[4.5]decan-1-yl)benzoic acid;
(R)—N-(1-(1-(1H-indol-5-yl)-3-methyl-2,4-dioxo-1,3,8-triazaspiro[4.5]decan-8-yl)-3-methyl-1-oxobutan-2-yl)-2-fluoro-5-(trifluoromethyl)benzamide;
4-((R))-8-((R)-2-(5-Ethyl-2-fluorobenzamido)-3-methylbutanoyl)-2,3-dimethyl-4-oxo-1,3,8-triazaspiro[4.5]decan-1-yl)benzoic acid; and
(R)—N-(1-(1-(1H-indazol-5-yl)-3-methyl-2,4-dioxo-1,3,8-triazaspiro[4.5]decan-8-yl)-3-methyl-1-oxobutan-2-yl)nicotinamide,
or a pharmaceutically acceptable salt thereof.

References to compounds of Formula I include compounds of Formula I, Ia, Id, Ih, Ik, Io, Ip, Iq, Ir, Is, It, Iu, Iv and Iw.

### IV. Pharmaceutical Compositions

The present invention includes pharmaceutical compositions of the compounds of Formula I and an additional therapeutic agent. In some embodiments, the present invention provides a pharmaceutical composition including a therapeutically effective amount of an autotaxin inhibitor compound of Formula I, or a pharmaceutically acceptable salt thereof, an additional therapeutic agent, and a pharmaceutically acceptable carrier or excipient.

The compounds useful in the pharmaceutical composition of the present invention include the compounds of Formula I, Ia, Id, Ih, Ik, Io, Ip, Iq, Ir, Is, It, Iu, Iv and Iw.

The pharmaceutical composition of the present invention can include one or more additional therapeutic agents. For example, the pharmaceutical composition can include 1, 2, 3, 4, 5, 6, or more, additional therapeutic agents. In some embodiments, the pharmaceutical composition include one additional therapeutic agent. In some embodiments, the pharmaceutical composition include two additional therapeutic agents. In some embodiments, the pharmaceutical composition include three additional therapeutic agents.

This disclosure provides pharmaceutical compositions that contain, as the active ingredient, one or more of the compounds of Formula I described above or a pharmaceutically acceptable salt or ester thereof and one or more pharmaceutically acceptable excipients, carriers, including inert solid diluents and fillers, diluents, including sterile aqueous solution and various organic solvents, permeation enhancers, solubilizers and adjuvants. The pharmaceutical compositions may be administered alone or in combination with other therapeutic agents (as indicated in the Combination Therapy section below). Such compositions are prepared in a manner well known in the pharmaceutical art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G.S. Banker & C.T. Rhodes, Eds.)

The pharmaceutical compositions may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously orally, topically, as an inhalant or via an impregnated or coated device such as a stent, for example or an artery-inserted cylindrical polymer.

One mode for administration is parenteral, particularly by injection. The forms in which the novel compositions of the present disclosure may be incorporated for administration by injection include aqueous or oil suspensions or emulsions, with sesame oil, corn oil, cottonseed oil or peanut oil, as well as elixirs, mannitol, dextrose or a sterile aqueous solution and similar pharmaceutical vehicles. Aqueous solutions in saline are also conventionally used for injection, but less preferred in the context of the present disclosure. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a compound according to the present disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the general methods of preparation are vacuum-drying and freeze-drying techniques which yield a powder of the active ingredient plus any additional desired ingredient from a previously sterile-filtered solution thereof.

Oral administration is another route for administration of compounds in accordance with the disclosure. Administration may be via capsule or enteric coated tablets or the like. In making the pharmaceutical compositions that include at least one compound described herein, the active ingredient is usually diluted by an excipient and/or enclosed within such a carrier that can be in the form of a capsule, sachet, paper or other container. When the excipient serves as a diluent, it can be in the form of a solid, semi-solid or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup and methyl cellulose. The formulations can additionally include: lubricating agents such as talc, magnesium stearate and mineral oil; wetting agents; emulsifying and suspending agents; preserving agents such as methyl and propylhydroxy-benzoates; sweetening agents; and flavoring agents.

The compositions of the disclosure can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present disclosure employs transdermal delivery devices (“patches”). Such transdermal patches may be used to provide continuous or discontinuous infusion of the compounds of the present disclosure in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile or on demand delivery of pharmaceutical agents.

In some embodiments, the compositions are formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. In some embodiments, each dosage unit contains from 1 mg to 2 g of a compound described herein and for parenteral administration, in some embodiments, from 0.1 to 700 mg of a compound a compound described herein. It will be understood, however, that the amount of the compound actually administered usually will be determined by a physician, in the light of the relevant circumstances, including the condition to be treated, the chosen route of administration, the actual compound administered and its relative activity, the age, weight and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present disclosure may be coated or otherwise compounded to provide a dosage form affording the advantage of prolonged action or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Compositions for inhalation or insufflation include solutions and suspensions in pharmaceutically acceptable, aqueous or organic solvents or mixtures thereof and powders. The liquid or solid compositions may contain suitable pharmaceutically acceptable excipients as described supra. In some embodiments, the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent or intermittent positive pressure breathing machine. Solution, suspension or powder compositions may be administered, in some embodiments orally or nasally, from devices that deliver the formulation in an appropriate manner.

In one embodiment, this disclosure relates to a pharmaceutical composition comprising a pharmaceutically acceptable excipient or carrier and a therapeutically effective amount of the compound of Compound I as described above or a pharmaceutically acceptable salt, ester, prodrug, stereoisomer or hydrate thereof.

The additional therapeutic agent can be any suitable therapeutic agent. For example, the additional therapeutic agent can be an anti-fibrotic agent, an oncology agent, an ASK-1 inhibitor, a cardiovascular agent, a SYK inhibitor, and others. In one embodiment, the additional therapeutic agent is an ASK-1 inhibitor. In one embodiment, the additional therapeutic agent is a SYK inhibitor. In one embodiment, the additional therapeutic agent is a LOXL2 inhibitor.

A. Anti-Fibrotic Agents

The present invention also includes a pharmaceutical composition of the present invention where the additional therapeutic agent can be an anti-fibrotic agent. In some embodiments, the present invention provides a pharmaceutical composition including a compound of Formula I, at least one additional therapeutic agent that is an anti-fibrotic agent, and a pharmaceutically acceptable carrier or excipient.

Anti-inflammatory agents useful in the present invention can be suitable to treat autoimmune and inflammatory diseases such as rheumatoid arthritis (RA), systemic lupus erythematosus (SLE), and asthma. Other diseases treatable with the anti-inflammatory agents include a fibrotic disease such as idiopathic pulmonary fibrosis.

In certain embodiments, a method for treating a fibrotic disease in a human having the fibrotic disease is provided, comprising administering to the human a therapeutically effective amount of a compound disclosed herein, or a pharmaceutically acceptable salt thereof, in combination with a therapeutically effective amount of one or more (e.g., one, two, three, one or two, or one to three) additional therapeutic agents. In certain embodiments, a method for treating an inflammatory disease in a human having the inflammatory disease is provided, comprising administering to the human a therapeutically effective amount of a compound disclosed herein, or a pharmaceutically acceptable salt thereof, in combination with a therapeutically effective amount of one or more (e.g., one, two, three, one or two, or one to three) additional therapeutic agents.

In one embodiment, pharmaceutical compositions comprising a compound disclosed herein, or a pharmaceutically acceptable salt thereof, in combination with one or more (e.g., one, two, three, one or two, or one to three) additional therapeutic agents, and a pharmaceutically acceptable carrier, diluent, or excipient are provided.

In certain embodiments, a compound disclosed herein, or a pharmaceutically acceptable salt thereof, is combined with one, two, three, four, or more additional therapeutic agents. In certain embodiments, a compound disclosed herein, or a pharmaceutically acceptable salt thereof, is combined with two additional therapeutic agents. In other embodiments, a compound disclosed herein, or a pharmaceutically acceptable salt thereof, is combined with three additional therapeutic agents. In further embodiments, a compound disclosed herein, or a pharmaceutically acceptable salt thereof, is combined with four additional therapeutic agents. The one, two, three, four, or more additional therapeutic agents can be different therapeutic agents selected from the same class of therapeutic agents, and/or they can be selected from different classes of therapeutic agents.

In certain embodiments, fibrotic diseases may include idiopathic pulmonary fibrosis (IPF), pulmonary fibrosis, interstitial lung diseases, nonspecific interstitial pneumonia (NSIP), usual interstitial pneumonia (UIP), endomyocardial fibrosis, mediastinal fibrosis, myelofibrosis, retroperitoneal fibrosis, progressive massive, nephrogenic systemic fibrosis, Grahn's disease, old myocardial infarction, scleroderma/systemic sclerosis, neurofibromatosis, Hermansky-Pudlak syndrome, diabetic nephropathy, renal fibrosis, hypertrophic cardiomyopathy (HGM), hypertension-related nephropathy, focal segmental glomerulosclerosis (FSGS), radiation-induced fibrosis, uterine leiomyomas (fibroids), alcoholic liver disease, hepatic steatosis, hepatic fibrosis, hepatic cirrhosis, hepatitis G virus (HGV) infection, chronic organ transplant rejection, fibrotic conditions of the skin, keloid scarring, Dupuytren contracture, Ehlers-Danlos syndrome, epidermolysis bullosa dystrophica, oral submucous fibrosis, and fibre-proliferative disorders, nonalcoholic steatohepatitis (NASH), alcoholic hepatitis, epidermolysis bullosa, dyskeratosis congenita, and Werner syndrome.

In certain embodiments, inflammatory disease may include chronic obstructive pulmonary disease, atopic dermatitis, anaphylactic shock fibrosis, psoriasis, allergic diseases, asthma, stroke, ischemia-reperfusion, platelets aggregation/activation, skeletal muscle atrophy/hypertrophy, leukocyte recruitment in cancer tissue, antiogenesis, invasion metastasis, melanoma, Karposi's sarcoma, acute and chronic bacterial and virual infections, sepsis, transplantation rejection, graft rejection, glomerulo sclerosis, glomerulo nephritis, progressive renal fibrosis, arthritis, rheumatoid arthritis, endothelial and epithelial injuries in the lung, and lung airways inflammation.

In the above embodiments, the additional therapeutic agents may be selected from hedgehog protein inhibitors, smoothened receptor antagonists, endothelin ET-A antagonists, endothelin ET-B antagonists, FGF receptor antagonists, FGF1 receptor antagonists, FGF2 receptor antagonists, PDGF receptor alpha antagonists, PDGF receptor antagonists, PDGF receptor beta antagonists, VEGF receptor antagonists, VEGF-1 receptor antagonists, VEGF-2 receptor antagonists, VEGF-3 receptor antagonists, IL-13 antagonists, interferon beta ligands, mTOR complex 1 inhibitors, TGF beta antagonists, p38 MAP kinase inhibitors, NADPH oxidase 1 inhibitors, NADPH oxidase 4 inhibitors, connective tissue growth factor ligand inhibitors, IL-6 antagonists, IL-6 agonists, insulin-like growth factor 1 antagonists, somatostatin receptor agonists, 5-lipoxygenase inhibitors, PDE 3 inhibitors, phospholipase C inhibitors, serum amyloid P stimulator, guanylate cyclase stimulator, PDE 4 inhibitors, Abl tyrosine kinase inhibitors, Kit tyrosine kinase inhibitors, signal transduction inhibitors, angiotensin II ligand modulator, endothelin 1 ligand inhibitors, relaxin agonist, IL-4 antagonist, TNF antagonist, type II TNF receptor modulator, monocyte chemotactic protein 1 ligand inhibitors, galectin-3 inhibitors, SH2 domain inositol phosphatase 1 stimulator, MAPKAPK2 inhibitors, caspase inhibitors, lysophosphatidate-1 receptor antagonist, beta 2 adrenoceptor agonist, interferon gamma ligands, superoxide dismutase modulator, hyaluronidase stimulator, transaminase stimulator, integrin alpha-V/beta-6 antagonist, a lysyl oxidase-like protein 2 (LOXL2) inhibitor, adrenoceptor antagonist, VIP agonist, interferon alpha ligands, phosphoinositide 3-kinase inhibitors, Jun N terminal kinase inhibitors, collagen V modulators, metalloprotease-9 stimulators, PPAR agonists, adenosine A2b receptor antagonists, GPCR modulators, CCR7 chemokine modulators, interleukin 17E ligand inhibitors, interleukin receptor 17B antagonists, AKT protein kinase inhibitors, hyaluronan mediated motility receptor modulators, angiotensin II AT-2 receptor agonists, CXC11 chemokine ligand modulators, immunoglobulin Fc receptor modulators, lysophosphatidate-1 receptor antagonists, ubiquitin thioesterase inhibitors, 5-HT 2b receptor antagonists, LDL receptor related protein-6 inhibitors, telomerase stimulators, endostatin modulators, Wnt-1 induced signal pathway protein 1 inhibitors, NK1 receptor antagonists, CD95 antagonists, protein tyrosine phosphatase 1E inhibitors, plasminogen activator inhibitors 1 inhibitors, spleen tyrosine kinase inhibitors, MMP9 inhibitors, TPL2 COT Kinase inhibitors, JAK1/2 inhibitors, Bruton's tyrosine kinase (BTK) inhibitors, integrin alpha 4 beta 7 inhibitors, PAD4 inhibitors, PAD2 inhibitors, IRAK4 inhibitors, ASK1 inhibitors, PIM1 inhibitors, PIM3 inhibitors, complement pathway inhibitors, AMPK inhibitors, IL-17 inhibitors, PD-1 agonist, IL-33 inhibtior, IL-25 inhibitors, and IL-22 agonists.

In certain embodiments, the additional therapeutic agents may be selected from vismodegib, macitentan, nintedanib, tralokinumab, ambrisentan, bosentan, interferon beta-1a, everolimus, GKT-137831, PBI-4050, PLX stem cell therapy (Pluristem/Cha Bio & Diostech), lanreotide, tipelukast, INT-0024, PRM-151, riociguat, roflumilast, imatinib, serelaxin, SAR-156597, etanercept, AEOL-10150, lebrikizumab, MPC-300-IV, FG-3019, carlumab, GR-MD-02, AQX-1125, MMI-0100, pirfenidone, deuterated pirfenidone analogs (e.g. SD-560), emricasan, Conatus, BMS-986020, beclometasone dipropionate+formoterol fumarate, TD-139, recombinant midismase, QAX-576, bovhyaluronidase azoximer, GNI/AFTF-351, BG-00011, simtuzumab, SPL-334, pentoxifylline+N-acetyl-cysteine, aviptadil, interferon-alpha, GSK-2126458, actimmune, bentamapimod, CKD-942, tanzisertib, interferon gamma, IW-001, PUR-1500, DB-029.01, disitertide, fresolimumab, IVA-337, PBF-1250, P-013, P-007, anti-IL-17BR humanized antibody, triciribine, RHAMM modulators, RES-529, MOR-107, hR-411, HEC-00000585, BOT-191, GKT-901, USP-34 inhibitors, anti-LRP6 mAb, Gestelmir, Neumomir, IBIO-CFB-03, MSM-735, LTI-03, anti-WISP1 antibodies, NAS-911B, C-301, STNM-04, TM-5441, PP-0612, QU-100, HR-017, Gal-100, MAI-100, BPS-03251, MMP9 antibodies, such as those disclosed in U.S. Pat. No. 8,377,443, ASK-1 inhibitors, such as those disclosed in U.S. Pat. No. 8,378,108, SYK inhibitors, such as those disclosed in US2015/0175616 and U.S. Pat. No. 8,450,321, for example, 6-(1H-indazol-6-yl)-N-(4-morpholinophenyl)imidazo[1,2-a]pyrazin-8-amine), inhibitors of Bruton's tyrosine kinase such as those disclosed in U.S. Pat. No. 8,557,803, for example, (R)-6-amino-9-(1-(but-2-ynoyl)pyrrolidin-3-yl)-7-(4-phenoxyphenyl)-7H-purin-8(9H)-one, FXR agonists such as those disclosed in US20140221659, and PI3K inhibitors, such as those disclosed in US20140371246.

### Hedgehog Protein Inhibitors

Examples of hedgehog protein inhibitors include glasdegib, ST-04464, necuparanib, ETS-2400, robotnikinin SHR-153, mifepristone derivatives, CEP-143, ISC-4, IMP-536, purmorphamine, BHM-427, patidegib, PF-0527485, and CD-05-002.

### Smoothened Receptor Antagonists

Examples of smoothened receptor antagonists include sonidegib, vismodegib, taladegib, glasdegib, XL-139, PI-722, patidegib, PF-05274857, MK-5710, LEQ-506, TAK-441, CD-05-002, and SMOi2-17.

### Endothelin ET-A Antagonists

Examples of endothelin ET-A antagonists include macitentan, ambrisentan, bosentan, atrasentan, sparsentan, zibotentan, PD-145065, fandosentan potassium, feloprentan, CPU-0213, sitaxentan, ABT-306552, clazosentan, TBC-3711, avosentan, PD-161721, BQ-153, BQ-123, darusentan, S-0139, 2-methoxyestradiol, TBC-3711, PD-156123, BMS-182874, BSF-461314, SB-234551, ZD-1611, 50-235, LU-127043, YM-62899, PD-163610, PD-142893, SB-209670, nebentan, Ro-61790C, ABT-546, PD-156707, BQ-610, Ro-48-5695, A-158112, T-0201, BE-18257B, A-207508, A-182086, SB-247083, EMD-122946, FR-139317, Ro-48-5694, TBC-10662, PD-160874, BQ-928, A-104029, A-203719, EMD-122801, SB-255757, PD-166673, BMS-187308, A-201661, SB-215355, PD-102566, PD-163070, EMD-94246, IRL-1543, RES 1214-1, BQ-518, PD-162073, PD-160672, PD-159020, PD-159433, FR-901367, PD-152884, and PD-155080.

### Endothelin ET-B Antagonists

Examples of endothelin ET-B antagonists include bosentan, PD-145065, BQ-788, feloprentan, CPU-0213, PD-161721, A-192621, Ro-46-8443, LU-127043, PD-142893, SB-209670, A-308165, K-8794, Ro-48-5695, A-158112, RES-701-1, A-182086, Ro-48-5694, PD-160874, BQ-928, BQ-017, IRL-1841, IRL-1722, CGP-49941, IRL-1543, RES-1149-1, PD-162073, PD-160672, PD-159020, IPI-950, and RES-701-2.

### Endothelin ET-B Antagonists

Examples of FGF receptor antagonists include CPL-043, nintedanib, BLU-554, masitinib, lenvatinib mesylate, ponatinib, lucitanib hydrochloride, regorafenib, FGFR2-ADC, BAY-1179470, regorafenib, LY-3076226, erdafitinib, FGF-401, squalamine, B-701, ENMD-2076, UCM-037, HMPL-453, sulfatinib, fenretinide, infigratinib, AZD-4547, alofanib, BAY-1163877, pirfenidone, FPA-144, RTEF-651, brivanib alaninate, dovitinib, Debio-1347, ARQ-087, OM-RCA-001, TAS-120, danusertib, ODM-203, S-49076, JNJ-42441707, INCB-054828, LY-2874455, ASP-5878, FP-1039, Loxo-103, PMX-20005, D-181, EDP-317, muparfostat sodium, AL-3818, AL-8326, ZLJ-33, KIN-4104, RG-7444, orantinib, LQN-725, Pantarin, PP-0612, AV-370, AV-369, K-983, BPS-03251, CT-400P, AM-001, PAT-PA1, TRC-093, DAPI-01, KW-2449, XL-999, ProMabin, PD-166285, EncaminC, SSR-128129, TG-100801, TBC-256, PD-089828, SU-9902, FCE-27164, and GMI-306.

### PDGF Receptor Antagonists

Examples of PDGF receptor antagonists include nilotinib, pazopanib, imatinib, X-82, nintedanib, masitinib, MG-516, DCC-2618, lenvatinib mesylate, Duta-101, olaratumab, ponatinib, lucitanib hydrochloride, pirfenidone, BLU-285, sorafenib, PK-10571, PK-453, axitinib, sunitinib, AR-13154, quizartinib dihydrochloride, cediranib, GFB-204, JI-101, dovitinib, XB-2202, ARQ-087, HLX-08, puquitinib mesylate, NT-506-ECT, famitinib, CLS-1002, KN-027, vatalanib, D-181, crenolanib, ilorasertib, AL-8326, AD-054.9, CG-026481, ZLJ-33, AbyD-3263, KN-014, orantinib, CS-2164, ARC-127, KBP-7018, AG-321, QLNC-3A6, tovetumab, amuvatinib, XV-615. mitothiorole, tandutinib, BMS-584622, ARRY-768, DCC-2157, XL-844, TAK-593, CP-673451, PD-166285, AMG-273, LY-2401401, LEO-A, GFB-111, CDP-860, AG-1295, RTKA-111, PD-089828, RPR-127963E, KI-6896, KI-6783, RPR-101511a, SU-65847, SU-65786, luteusin-C, WIN-41662, and CGP-53716.

VEGF Receptor Antagonists

Examples of VEGF receptor antagonists include apatinib mesylate, pazopanib, ranibizumab, DCB-R0237, X-82, MGCD-265, nintedanib, cabozantinib, vandetanib, altiratinib, MG-516, ramucirumab, lenvatinib mesylate, Duta-101, ponatinib, conbercept, PZ-1, anlotinib hydrochloride, lucitanib hydrochloride, sorafenib, STI-A0168, regorafenib, fruquintinib, NT-503-ECT, regorafenib, axitinib, pegaptanib, PAN-90806, sunitinib, RGX-314, tivozanib, ENMD-2076, UCM-037, cediranib, sulfatinib, GFB-204, AFG-2, JI-101, BNC-420, brivanib alaninate, dovitinib, TAS-115, TTAC-0001, LCB-19, GNR-011, DA-3131, IMC-3C5, HLX-06, rebastinib, motesanib diphosphate, ODM-203, AG-119, PSI-001, famitinib, CLS-1002, DE-120, KN-027, ningetinib, OMP-305B83, Debio-1144, LAU-0901, foretinib, WXH-520, DIG-KT, CYC-116, sevacizumab, APX-004, PMX-20005, vatalanib, D-181, elpamotide, OTSGC-A24, DP-317, UB-924, muparfostat sodium, Angiozyme, ilorasertib, AL-2846, AL-3818, BMS-817378, AL-8326, PTC-299, PRS-050, UBP-1212, RAF-265, CEP-11981, CG-203306, A-1014907, MDX-1, WS-006, ZLJ-33, ABS-393, S-209, MP-0250, KIN-4104, TLK-60404, KN-014, SAR-397769, SAR-131675, CS-3158, golvatinib tartrate, ABT-165, OSI-930, orantinib, icrucumab, PLG-201, PLG-101, BGB-102, squalamine, CS-2164, AR-639, NX-278-L, KBP-7018, IBI-302, AG-321, BFH-772, AD-051.4, TAK-632, IPS-04003, QLNC-3A6, IPS-04001, AP-202, LP-590, telatinib, SCR-1515, BRN-103, LMV-12, PTZ-09, ENMD-1198, ACTB-1011, 4SC-203, AS-3, IXS-312, linifanib, MRC-202, XV-615, mitothiorole, IMC-1C11, NT-502, pegdinetanib, ESBA-903, GSK-2136773, KRN-633, BMS-584622, PF-337210, SA-20896, alacizumab pegol, CLT-007, CLT-006, ZK-261991, SU-14813, DCC-2157, XL-999, BMS-690514, TAK-593, NM-3, PRS-055, AMG-273, BIW-8556, BMS-645737, DMS-3008, NSTPBP-01250, C11C1, EG-3306, AAL-881, AE-941, semaxanib, LY-2401401, OSI-632, Hu2C3, LEO-A, BIBF-100, AG-028262, TX-2036, GFB-111, AB-434, EHT-0204, RG-8803, ZD-4190, ZK-304709, HuMV833, AG-013958, L-000021649, AZD-9935, JNJ-17029259, DX-1235, AG-28345, AG-28191, VGA-1102, R-123942, CEP-5214, KP-0201448, ZK-229561, TBC-2576, CHIR-200131, KM-2550, TG-100-344, AG-13925, LU-343505, TBC-1635, SU-9902, SU-9803, SU-4158, NX-213, SoRI-8790.

### IL-13 Antagonists

Examples of IL-13 antagonists include tralokinumab, lebrikizumab, VBP-15, dupilumab, RPC-4046, SAR-156597, MEDI-7836, AZD-0449, CDP-7766, ASLAN-004, anrukinzumab, CNTO-5825, GSK-2434735, AIR-645, CNTO-607, IMA-026, AMG-317, RG-1671, and DOM-1000P.

mTOR Complex 1 Inhibitors

Examples of mTOR complex 1 inhibitors include VS-5584, ABTL-0812, vistusertib, sapanisertib, DS-3078, CC-223, SF-1126, PQR-309, dactolisib, apitolisib, GSK-2126458, OSI-027, CC-214, AZD-8055, BI-860585, XL-388, and OXA-01.

### TGF Beta Antagonists

Examples of TGF beta antagonists include luspatercept, pirfenidone, dalantercept, ASPH-1106, DB-029.01, ACE-083, CAR-decorin, fresolimumab, Actimmune, galunisertib, ASPH-0047, trabedersen, ASPH-1047, BG-00011, NCE-401, ARGX-115, TEW-7197, WilVent, r150, YH-14618, P-2745, YH-14619, ACE-661, PTL-101, NX-027, DRP-049, ACE-435, SMP-534, HSc-025, decorin, A-77, SB-525334, ANG-1122, metelimumab, LF-984.

### p38 MAP Kinase Inhibitors

Examples of p38 MAP kinase inhibitors include losmapimod, pirfenidone, VX-745, TG-02, BCT-197, ARRY-797, AZD-7624, RV-568, AM-102, MW-108, Minokine, ralimetinib, FX-005, pamapimod, UR-13870, UR-5269, RO-320-1195, PHA-00797804, pexmetinib, PH-797804, RV-1088, AKP-001, LP-590, PF-994888, LY-3007113, LASSBio-998, AMG-548, AW-814141, dilmapimod, CHR-3620, LP-1890, SCIO-323, GSK-725, RDP-58, GSK-610677, BMS-626531, BMS-582949, talmapimod, SB-203580, R-1487, doramapimod, TAK-715, AZD-6703, VX-702, SB-239063, TA-5493, HEP-689, SB-220025, RWJ-67657, ABC-1, LEO-15520, SCR-0265096, ARQ-101, KC-706, SC-80036, SB-281832, SB-239065, AVE-9940, SC-XX906, SB-238039, RPR-200765A, SC-040, and CP-64131.

### NADPH Oxidase Inhibitors

Examples of NADPH oxidase inhibitors include GKT-901, GKT-137831, NV-196, ME-143, Phox-I, shikonin, and VAS-2870.

### Connective Tissue Growth Factor Ligand Inhibitors

Connective tissue growth factor ligand inhibitor: netarsudil, PBI-4050, RXI-109, FG-3019, ALT-0701, and PBI-4419.

### IL-6 Agonists

Examples of IL-6 agonists include atexakin alfa, gludapcin, and DAB389-IL-6.

### Insulin-Like Growth Factor Antagonists

Examples of insulin-like growth factor antagonists include lanreotide, ganitumab, BI-836845, dusigitumab, NT-219, MM-141, linsitinib, ATL-1101, AZD-3463, ANT-429, FP-008, PL-225B, dalotuzumab, robatumumab, BMS-754807, cixutumumab, IG01A-048, KW-2450, XL-228, GTx-134, A-923573, AD-0027, INT-231, GSK-1904529A, A-928605, AEW-541, figitumumab, PQIP, AVE-1642, A-947864, BIIB-022, h10H5, KM-1468, PNU-145156E, and AG-1024.

### Somatostatin Receptor Agonists

Examples of Somatostatin receptor agonists include pasireotide, PTR-3173, lanreotide, G-0211, FP-002, SomaDex, TLN-232, RFE-114, RFE-011, RFE-107, BIM-23190, FK-962, L-363377, NNC-26-9100, FK-960, LAN-7, vapreotide, seglitide, SDZ-221-047, BIM-23027, ilatreotide, and BIM-23034.

### 5-Lipoxygenase Inhibitors

Examples of 5-Lipoxygenase inhibitors include JRP-980, JRP-890, tipelukast, ML-4000, tenoxicam, TA-270, AC-225, Q-501, darbufelone, Neu-164, zileuton, setileuton, ZLJ-6, KRH-102140, tebufelone, rilopirox, MK-5286, atreleuton, CJ-13610, PF-4191834, WY-50295-tromethamine, A-7917, licofelone, veliflapon, R-zileuton, MK-886, ZD-2138, etalocib, nicaraven, linazolast, BAY-U-9773, ON-09300, tenidap, LDP-392, PEP-03, NIK-639, BMD-188, BOM-1006, S-19812, tepoxalin, FPL-64170, AZD-4407, docebenone, UCB-35440, BW-B70C, flobufen, CBS-113-A, MK-866, PD-146176, CV-6504, ZD-2138 analogs, SKF-86002, R-68151. LY-221068. CMI-206, piriprost, bunaprolast, SC-45662, SC-41661A, PF-5901, ETH-615, SB-210661, PGV-20229, ZD-4007, ER-34122, FR-122788, L-705302, A-121798, PD-089244, E-6080, CMI-568, L-697198, RWJ-63556, L-70878, 3323W, ICI-211965, E-6700, BW-A4C, BW-A137C, P-10294, HX-0836, A-72694, FR-110302, L-739010, VZ-564, WY-28342, ONO-LP-049, L-702539, CGS-25997, HN-3392, R-840, BF-389, T-0757, T-0799, WAY-127153, WAY-126241, SKF-107649, WAY-126299A, KC-11425, KC-11404, ZM-216800, PD-145246, WAY-125007, ZD-7717, BW-755-C, BW-858C, BW-862C, L-699333, E-3040, ZM-230487, CGS-26529, A-63162, lagunamycin, PD-136005, BAY-Q-1531, L-651896, L-656224, CGS-23885, BU-4601A, LY-280810, SKF-104351, L-691816, A-69412, nitrosoxacin-A, epocarbazolin-A, WAY-120739, Sch-40120, SB-202235, P-8977, P-8892, LY-233569, lonapalene, L-674636, L-670630, enazadrem, DuP-654, CI-986, CI-922, CGS-22745, carbazomycin B, BI-L-357, tagorizine, A-80263, and SKF-105809.

### PDE3 Inhibitors

Examples of PDE3 inhibitors include anagrelide, tipelukast, RPL-554, cilostazol, milrinone, parogrelil, K-134, Thromboreductin, CR-3465, Kyorin, rafigrelide, pimobendan, LASSBio-294, NSP-513, SKF-95654, siguazodan, ATI-22107, olprinone, SKF-94120, flosequinan, Org-30029, K-123, hydroxypumafentrine, AWD-12-250, OPC-33540, TZC-5665, tolafentrine, MS-857, revizinone, Org-9935, KF-15232, pumafentrine, WIN-62582, nanterinone, CCT-62, Org-9731, EMD-57439, EMD-53998, WIN-62005, WIN-58993, WIN-63291, Org-20494, NSP-307, FK-664, NSP-306, SDZ-ISQ-844, SDZ-MKS-492, Org-20241, 349U85, and LAS-31180.

### Phospholipase C Inhibitors

Examples of Phospholipase C inhibitors include tipelukast, LMV-601, VLCA-04, U-73122, D-609, CPR-1006, D-20133, hispidospermidin, and CRM-51005.

### PDE 4 Inhibitors

Examples of PDE 4 inhibitors include apremilast, tipelukast, RPL-554, roflumilast, T-094, Hemay-005, crisaborole, AN-2898, CC-11050, BAL-0105277, ABI-4, DRM-02, HPP-737, LASSBio-596, tetomilast, TAK-648, LAS-37779, CHF-6001, CD-160130, OCID-2987, AVE-8112, HT-0712, UR-5908, E-6005, ASP-3258, PXSTPI-1100, OPA-15406, TA-7906, M-5200, NCS-613, GSK-356278, etazolate, INDUS-82010, AN-6415, BYK-321084, revamilast, GEBR-7b, catramilast, CR-3465, GPD-1116, AL-59640, TAS-203, elbimilast, ZL-N-91, MEM-1414, MK-0873, oglemilast, ASP-9831, cilomilast, YM-393059, rolipram, CC-1088, RBx-10017876, MK-0952, OX-914, DE-103, ND-1510, ND-1251, lirimilast, GP-0203, UCB-101333-3, Ro-20-1724, atizoram, cipamfylline, MEM-1917, KF-19514, tofimilast, GRC-3590, Org-30029, CDP-840, Sch-351591, CH-4139, CH-2874, CH-3442, CH-3697, D-4418, 4AZA-PDE4, ELB-526, arofylline, XT-611, KW-4490, ONO-6126, CC-7085, YM-976, GRC-3566, hydroxypumafentrine, piclamilast, GRC-3015, BAY-61-9987, R-1627, GRC-3785, V-11294A, WAY-127093B, filaminast, CDC-998, daxalipram, NIK-616, tolafentrine, RPR-122818, D-22888, CT-5357, AP-0679, doxofylline, CP-353164, pumafentrine, RPR-132294, Org-9731, RPR-117658, CP-293321, CP-146523, RPR-114597, GW-3600, E-4021, WAY-122331, XT-044, SDZ-PDI-747, WAY-126120, YM-58997, SKF-107806, PDB-093, CH-928, CH-673, CH-422, SDZ-ISQ-844, and Org-20241.

### Abl Tyrosine Kinase Inhibitors

Examples of Abl tyrosine kinase inhibitors include nilotinib, imatinib, PF-30, SX-004, bosutinib, dasatinib, ABL-001, selinexor, radotinib, bafetinib, rebastinib, saracatinib, danusertib, HS-543, BEBT-201, AN-019, CU-201, flumatinib, ORB-0001, SUN K-706, PF-114, ON-044580, NPB-001-056, XL-228, adaphostin, SGX-393, 18F-SKI-696, A-419259, EBI-600398, DCC-2157, and KW-2449.

### Kit Tyrosine Kinase Inhibitors

Examples of Kit tyrosine kinase inhibitors include KTN-0158, ganetespib, lenvatinib mesylate, nilotinib, pazopanib, imatinib, cabozantinib, 1J-373, masitinib, MG-516, DCC-2618, ponatinib, lucitanib hydrochloride, dasatinib, BLU-285, sorafenib, regorafenib, midostaurin, sunitinib, tivozanib, pexidartinib, quizartinib dihydrochloride, cediranib, dovitinib, rebastinib, motesanib diphosphate, NMS-088, famitinib, D-181, AL-8326, ZLJ-33, OSI-930, KBP-7018, QLNC-3A6, telatinib, CK-6, ACTB-1011, WBZ-7, amuvatinib, linifanib, tandutinib, ITRI-227, AMG-191, VX-322, KRN-633, ZK-261991, DCC-2157, XL-999, AMG-273, LY-2401401, LEO-A, MP-371, EXEL-0862, XL-820.

### Signal Transduction Inhibitors

Examples of signal transduction inhibitors include imatinib, NV-196, APC-300, APC-100, CPC-507, CB-1107, AEZS-127, HM-610368, CPR-1006, and KRX-0404.

### Angiotensin II Ligand Modulators

Examples of Angiotensin II ligand modulators include LJPC-501 and srelaxin.

### Relaxin Agonists

Examples of relaxin agonists include ANG-4011, serelaxin, ARX-720, and CGEN-25009.

### IL-4 Antagonists

Examples of IL-4 antagonists include dupilumab, SAR-156597, PRS-060, RNS-60, Actimmune, MDNA-11, MDNA-57, MDNA-56, MDNA-55, AZD-0449, TAQ-588, pascolizumab, GSK-2434735, AIR-645, AVE-0309, suplatast tosilate, AMG-317, TMC-256C1, D-22558.

### TNF Antagonists

Examples of TNF antagonists include SAR-244181, denosumab, etanercept, brentuximab vedotin, AVX-470, BIIB-023, fulranumab, tanezumab, GBR-830, AG-014, lucatumumab, fasinumab, BI-655064, BN-006, ASKP-1240, RNS-60, APG-101, PF-688, APX-005M, ONL-1204, AFM-13, FFP-104, RPH-203, MEDI-578, mDTA-1, AVX-1555, TDI-00846, IDD-004, APX-008, NM-9405, FFP-102, DS-8273, KGYY-15, ONL-101, SCB-808, SCB-131, Atu-614, DE-098, FFP-106, p75NTR-Fc, ANA-02, MEDI-4920, Novotarg, BMS-986090, VAY-736, CD40DNA Vax, GSK-2800528, pegsunercept, GBL-5b, NM-2014, Neutrolide, K-252a, ATROSAB, ABT-110, SAR-127963, 5C-11, ACE-772, ISIS-22023, CRB-0089, oxelumab, enavatuzumab, ALD-906, VT-362, F45D9, F61F12, ALD-901, AMPTIRA, APG-103, E-3330, dacetuzumab, rolipram, AG-879, onercept, D-609, DE-096, EC-234, MDX-1401, BIIB-036, ALS-00T2-0501, CZEN-001, P-60 PLAD, PD-90780, LT-ZMP001, CS-9507, PCM-4, toralizumab, DOM-0100, ReN-1820, solimastat, iratumumab, CGEN-40, PN-0615, lenercept, AUX-202, DOM-0800, ITF-1779, CEP-751, daxalipram, B-975, teneliximab, ALE-0540, MDL-201112, and BB-2275.

### Type II TNF Receptor Modulators

Examples of type II TNF receptor modulators include etanercept.

### Monocyte Chemotactic Protein 1 Ligand Inhibitors

Examples of monocyte chemotactic protein 1 ligand inhibitors include MRX6, carlumab, bindarit, MW01-2-15-ISRM, NN-8209, HMPL-011, BL-2030, CGEN-54, C-242, BKT-P46, and ABN-912.

### Galectin-3 Inhibitors

Examples of galectin-3 inhibitors include ANG-4021, GR-MD-02, LJPC-301, LJPC-201, TD-139, TFD-100, LJPC-1010, GR-MD-03, Gal-200, Galectin-3C, GM-CT-01, Gal-100, GM-MD-01, and GM-CT-02.

### SH2 Domain Inositol Phosphatase 1 Stimulators

Examples of SH2 domain inositol phosphatase 1 stimulators include AQX-1125 and AQX-MN-100.

### MAPKAPK2 Inhibitors

Examples of MAPKAPK2 inhibitors include MMI-0100, CDD-11, and SCR-0265096.

### Caspase Inhibitors

Examples of caspase inhibitors include DPT-PEP1, F-573, CVXL-0103, NWL-53, NWL-117, YJP-60107, DCP-LA, nivocasan, IDN-7314, VX-166, LFM-A12, LFM-A13, prainacasan, VX-799, IDN-1965, IDN-6734, L-709049, MX-1122, Tan-1756A, TLC-144, SDZ-224-015, EI-1507-1, SB-234470, and SDZ-220-976.

### Lysophosphatidate-1 Receptor Antagonists

Examples of lysophosphatidate-1 receptor antagonists BMS-986020, SAR-100842, and Debio-0719.

### Beta 2 Adrenoceptor Agonists

Examples of beta 2 adrenoceptor agonists include arformoterol, salbutamol, indacaterol, sibenadet, AR-C-89855, picumeterol, R-salmeterol, LM-2616, RP-58802B, batefenterol succinate, vilanterol, formoterol, olodaterol, abediterol, AZD-8999, AZD-2115, bambuterol, TD-5471, bedoradrine, AZD-3199, milyeterol, KUL-7211, EP-102, PF-3429281, broxaterol, indacaterol xinofoate, CRx-501, carmoterol, PF-610355, ASF-1020. GSK-597901, Meluadrine, NCX-950, S-1319, KUR-1247, KUL-1248, AR-C-89855, picumeterol, LM-2616, and RP-58802B.

### Superoxide Dismutase Modulators

Examples of superoxide dismutase modulators include GC-4419, midismase, calmangafodipir, decuprate, NUCC-434, VY-SOD-101, NI-204A, APN-201, imisopasem manganese, EUK-207, M-101, pegorgotein, MTS-01, HG-1163, RTA-801, M-40401, SC-65224, SC-55858, SC-52608, and CDRI-81-470.

### Integrin Alpha-V/Beta-6 Antagonists

Examples of integrin alpha-V/beta-6 antagonists include BG-00011, IK-248, A20FMDV2, and intelumumab.

### Lysyl Oxidase Homolog 2 Inhibitors

Examples of lysyl oxidase homolog 2 inhibitors include simtuzumab and AB0023.

### VIP Agonists

Examples of VIP agonists include PB-1046, Eu-111, LBT-3627, RG-7103, AR-D-111421, and Ro-25-1553.

### Phosphoinositide 3-Kinase Inhibitors

Examples of Phosphoinositide 3-kinase inhibitors include buparlisib, neratinib, duvalisib, LY-3023414, gedatolisib, IPL-549, VS-5584, IBL-301, IBL-202, pictilisib, X-414, TGR-1202, X-339, X-480, idelalisib, sirolimus, AMG-319, TAM-01, PWT-143, ME-344, CC-115, ZSTK-474, alpelisib, HL-156A, CHY-33, CDZ-173, AZD-8835. AEZS-136, ARQ-092, BEBT-908, copanlisib, TAK-117, DS-7423, temsirolimus, perifosine, XL-499, taselisib, SF-1126, INCB-40093, RV-1729, GS-9820, PQR-309, ASN-003, CUDC-907, pilaralisib, dactolisib, PBI-05204, SAR-260301, CLR-1502, AEZS-129, AMG-511, AZD-8186, RP-6530, PF-4691502, KBP-7306, INCB-50465, voxtalisib, GSK-2269557, GSK-2636771, apitolisib, GS-9829, BAY-1082439, TP-3654, CT-365, KA-2237, FP-208, BEBT-906, PQR-514, PQR-401, PQR-620, PQR-530, CT-732, WX-037, GSK-2126458, PQR-5XX, KAR-4141, HMPL-689, UCB-5857, IPI-443, GS-9901, OB-318, RG-7666, ridaforolimus, CAL-130, CNX-1351, Rapatar, X-370, panulisib, OSI-027, ON-123300, NV-128, HS-113, SMI-4a, RP-6503, LAS-194223, CLR-457, LS-008, RP-5090, SRX-2523, SRX-2626, SRX-5000, SF-2535, SF-2558HA, INK-007, GSK-418, VDC-597, PA-799, Triflorcas, CL-27c, SRX-2558, BN-107, SKLB-JR02, EC-0371, PKI-402, PQR-316, PQR-311, Y-31, PQR-370, PQR-340, PQR-312, CU-906, OXA-01, GAP-107B8, EC-0565, ONC-201, P-6915, AZD-6482, EM-101, GDC-0349, X-387, TAFA-93, WJD-008, CLR-1401, RP-5002, LY-294002, P-2281, AQX-MN100, PKI-179, CAL-263, BGT-226, QLT-0447, CHR-4432, BAG-956, EM-12, GSK-1059615, AQX-MN106, and PX-867.

### Jun N Terminal Kinase Inhibitors

Examples of Jun N terminal kinase inhibitors include bentamapimod, CC-90001, AX-14373, JNK-401, XG-102, JNK-IN-8, IT-139, tanzisertib, AIK-2, SR-3306, PG-11144, AEG-33783, SPC-9766.

### Collagen V Modulators

Examples of collagen V modulators include IW-001 and TRC-093.

### PPAR Agonists

Examples of PPAR agonists include pioglitazone, K-877, rosiglitazone, KPT-350, troglitazone, SER-150-DN, MBX-8025, INDUS-810, T3D-959, IVA-337, efatutazone, saroglitazar, CER-002, elafibranor, KDT-501, HPP-593, OMS-405, bezafibrate, CXR-1002, INT-131, aleglitazar, BPM-18708, D-9091, ATx08-001, FP-0250, IDR-105, CDE-001, VCE-004.8, THR-0921, lobeglitazone, CS-038, DSP-8658, AVE-0897, IDB-101, ALL-4, KY-903, tesaglitazar, KDT-500, CLC-3001, rosiglitazone XR, indeglitazar, DJ-5, KR-62980, RSC-451061, balaglitazone, ZBH-2011-02, darglitazone, KD-3010, AZD-4619, LY-554862, PRB-2, MP-136, rivoglitazone, DB-900, KRP-105, GW-409544, KRP-101, muraglitazar, FK-614, GW-2433, GW-2331, AD-5075, edaglitazone, PAM-1616, GW-501516, DRL-17564, DRF-11605, DRF-10945, MK-0533, SAR-351034, farglitazar, DRL-15609, DRF-2519, TY-51501, NS-220, RWJ-667567, 625019, KRP-297, reglitazar, K-111, LY-674, GSK-376501, MBX-2599, MBX-213, ragaglitazar, AVE-8134, naveglitazar, oxeglitazar, netoglitazone, SDX-101, AKP-320, cevoglitazar, GW-590735, etalocib, KT6-207, E-3030, RG-12525, sodelglitazar, L-165041, PA-082, AVE-0847, GFT-14, DRF-4158, NIP-223, NIP-221, LY-929, ONO-5129, DRF-4832, CLX-0940, DRF-2189, CS-204, EML-2949, spirolaxine, GW-7845, peliglitazar, AVE-5376, NC-2100, imiglitazar, VDO-52, SBR-111895, LG-100754, GW-1536, AR-H049020, englitazone, SB-219994, LY-300512, GW-409890, AHG-255, LY-282449, AY-31637, SB-213068, BM-13.1246, R-102380, and YM-268.

### Adenosine A2b Receptor Antagonists

Examples of Adenosine A2b receptor antagonists include PBF-1350, PBF-1250, GS-6201, ATL-844, E-3210, PNQ-201, PNQ-103, ATL-801, LAS-101057, LUF-5451, MRS-1595, CMB-6446.

### Interleukin 17 Ligand Inhibitors

Examples of interleukin 17 ligand inhibitors include RG-7624, COVA-322, ABT-122, bimekizumab, CJM-112, and RG-4934.

### Interleukin Receptor 17 Antagonists

Examples of interleukin receptor 17 antagonists include brodalumab, secukinumab, SR-2211, ixekizumab, M-1095, KD-025, AFB-035, IMO-3100, vidofludimus, BCD-085, ANB-004, OREG-203, EBI-028, PRS-190, COVA-302, and CAT-2200.

### AKT Protein Kinase Inhibitors

Examples of AKT protein kinase inhibitors include JRP-980, JRP-890, CF-102, ipatasertib dihydrochloride, TX-803, CC-115, ONC-201, ONC-212, AZD-5363, AT-13148, M-2698, ARQ-092, afuresertib, perifosine, UCN-01, MK-2206, ALM-301, PQR-309, COTI-2, ASP-8273, CLR-1502, AMG-511, AR-12, NU-1001-41, TAS-117, BAY-1125976, ARQ-751, GSK-2636771, LY-2780301, TP-3654, PQR-401, OB-318, SR-13668, IMB-YH-8, VLI-27, AV-203, PHT-427, Triflorcas, MK-8156, SZ-685C, GSK-2334470, LD-101, XL-418, CLR-1401, LY-2503029, GSK-690693, PX-316, BAG-956, and EM-12.

### Angiotensin II AT-2 Receptor Agonists

Examples of Angiotensin II AT-2 receptor agonists include MOR-107, MP-157, and C21.

### CXC11 Chemokine Ligand Modulators

Examples of CXC11 chemokine ligand modulators include hR-411 and HG-1096.

### Immunoglobulin Fc Receptor Modulators

Examples of immunoglobulin Fc receptor modulators include Epsi-gam, GFD, SCIB-1, SIF-3, AFM-21, Dibegone, NPT-088, GL-2045, CST-103, HL-161, SM-211, SM-301, SM-201, SM-101, NT-P-01, NT-CP-02, AFM-13, AHG-2, RPH-203, R-421, hR-411, BI-1206, MGD-010, MDX-33, ertumaxomab, AZ-175, INA-02, AFM-12, ACE-661, HF-1020, PF-4605412, DX-2500, TTI-314, Y175L, ALKS-6931, HG-1206, HG-1205, GMA-161. MGA-321, GMR-321, TI-3, MDX-214, and AVI-073.

### Lysophosphatidate Receptor Antagonists

Examples of lysophosphatidate receptor antagonists include MT-1303. BMS-986020, SAR-100842, ONO-1266, sonepcizumab, NOX-S93, EDD7H9, Debio-0719, XL-541, and VPC-51299.

### Ubiquitin Thioesterase Inhibitors

Examples of ubiquitin thioesterase inhibitors include VLX-1570, P005091, and P22995.

### 5-HT 2b Receptor Antagonists

Examples of 5-HT 2b receptor antagonists include AM-1030, RQ-00310941, piromelatine, AMAP-102, BF-1, ER-21027, PRX-8066, vabicaserin, F-16615, SB-200646A, LY-266097, Terguride, LY287375, MT500, SB-206553, SB-221284, LY272015, and SDZ-SER-082.

### LDL Receptor Related Protein Modulators

Examples of LDL receptor related protein modulators include Wnt-001, CLT-020, MT-007, paclitaxel trevatide, NT-1654, ANG-2002, and NU-206.

### Telomerase Stimulators

Examples of telomerase stimulators include telanmir, gestelmir, DOSmir, Parmir, Myomir, Anemir, Neumomir, TAT-0002, GRN-510, and GRN-139925.

### Endostatin Modulators

Examples of endostatin modulators include EncorStat®, RetinoStat®, EDS-01, E-10A, EBIO-CFB-03, M2ES, P-1000, PC-24, SIM-0702

### NK1 Receptor Antagonists

Examples of NK1 receptor antagonists include aprepitant, fosaprepitant, tradipitant, HTX-019, netupitant, serlopitant, orvepitant, NAS-911B, ZD-6021, KD-018, DNK-333, NT-432, NK-949, NT-814, EU-C-001, vestipitant, 1144814, SCH-900978, AZD-2738, BL-1833, casopitant, AV-810, KRP-103, 424887, cizolirtine, vofopitant, L-742694, capsazepine, GR-82334, MEN-11149, L-732138, NiK-004, TA-5538, CP-96345, lanepitant, LY-2590443, dapitant, burapitant, befetupitant, CJ-17493, AVE-5883, CGP-49823, CP-122721, CP-99994, SLV-317, TAK-637, L-733060, dilopetine, MPC-4505, L-742311, FK-888, WIN-64821, NIP-530, SLV-336, ezlopitant, TKA-457, figopitant, ZD-4794, CP-100263, GR-203040, L-709210, MEN-10930, MEN-11467, LY-306740, FK-355, WIN-67689, WIN-51708, FK-224, BL-1832, CAM-6108, CP-98984, WS-9326A, L-741671, L-737488, L-740141, L-161664, YM-49244, Sch-60059, SDZ-NKT-343, S-18523, RPR-111905, S-19752, L-161644, LY-297911, RPR-107880, L-736281, anthrotainin, RP-73467, WIN-64745, WIN-68577, WIN-66306, RP-67580, CP-0364, L-743986, S-16474, CGP-47899, FR-113680, YM-44778, GR-138676, CGP-73400, CAM-2445, MDL-105172A, L-756867, isbufylline, and CP-0578

### CD95 Antagonists

Examples of CD95 antagonists include APG-101, ONL-1204, ONL-101, Atu-614, DE-098, Novotarg, ISIS-22023, F45D9, F61F12, APG-103, CS-9507

### Plasminogen Activator Inhibitor 1 Inhibitors

Examples of plasminogen activator inhibitor 1 inhibitors include BST-2006, THR-18, TM-5441, IMD-4482, IMD-4852, IMD-1041, and IMD-1622

### Spleen Tyrosine Kinase Inhibitors

Examples of spleen tyrosine kinase inhibitors include TAS-5567, fostamatinib, TAK-659, entospletinib, HMPL-523, AB-8779, cerdulatinib, PRT-2761, GS-9876, GSK-2646264, PRT-2607, CVXL-0102, CVXL-0101, CVXL-0074, R-348, PRT-060318, CC-485118, R-391, R-333, UR-67767, DNX-2000, R-343, CC-509, CG-103065, R112, R-280, AVE-0950, and ER-27319

### Bruton's Tyrosine Kinase Inhibitors

Examples of Bruton's tyrosine kinase inhibitors include (S)-6-amino-9-(1-(but-2-ynoyl)pyrrolidin-3-yl)-7-(4-phenoxyphenyl)-7H-purin-8(9H)-one, ibrutinib, HM71224, ONO-4059, spebrutinib (CC-292), acalabrutinib (ACP-196), PRN-1008, BGB-3111, TAK-020, M-2951, dasatinib, M-2951, HCL-1401, HM-71224, PRN-1008, TAS-5315, BGB-3111, AS-550, DR-109, TAK-020, SNS-062, ONO-4059, X-022, TP-4207, KBP-7536, GDC-0834, ONO-WG-307, and LFM-A13.

### MMP9 Inhibitors

Examples of MMP9 inhibitors include marimastat (BB-2516), cipemastat (Ro 32-3555), DP-b99, AZD-1236, SP-8203, LAU-0901, NM-AQU-005, SI-1005, SI-1004, tigapotide, DX-2802, CG-2608, CG-2575, CG-2507, IBFB-120082, AE-941, galarubicin, ABT-518, KT5-12, MMI-166, and RS-113456

### Janus Kinase Inhibitors

Examples of Janus Kinase inhibitors (e.g. JAK1 and JAK2) include ABT-494, filgotinib, ganetespib, tofacitinib, PF-04965842, ruxolitinib, pacritinib, CF-102, momelotinib, baricitinib, CS-944X, AT-9283, TG-02, AR-13154, ENMD-2076, VR-588, YJC-50018, INCB-39110, NS-018, GLPG-0555, G5-7, BVB-808, INCB-52793, fedratinib, PF-06263276, TP-0413, INCB-47986, CT-1578, peficitinib, BMS-911543, XL-019, solcitinib, MRK-12, AC-410, NMS-P953, CPL-407-22, CPL-407-105, AZD-1480, gandotinib, INCB-016562, CEP-33779, ON-044580, lestaurtinib, K-454, LS-104, SGI-1252, and EXEL-8232.

### Integrin Alpha-4/Beta-7 Antagonists

Examples of integrin alpha-4/beta-7 antagonists include PTG-100, AJM-300, etrolizumab, TRK-170, and abrilumab.

### IRAK Protein Kinase Inhibitors

Examples of IRAK protein kinase inhibitors include PF-06650833 and HU-003.

### Apoptosis Signal-Regulating Kinase (ASK) Inhibitors

Examples of apoptosis signal-regulating kinase (ASK) inhibitors (e.g. MEKK-5, ASK1) include ARN-7016, KC-459, CS-410, and SRI-28731. ASK inhibitors include ASK1 inhibitors. Examples of ASK1 inhibitors include, but are not limited to, those described in U.S. 2011/0009410 (Gilead Sciences) and U.S. 2013/0197037 (Gilead Sciences), as more fully set forth below.

### PIM Protein Kinase Inhibitors

Examples of PIM protein kinase inhibitors include: SEL-24, IBL-301, PIM-447, IBL-202, SEL-24B, SF-1126, ON-108600, AZD-1208, TP-3654, CXR-1002, ON-108110, SRX-2523

### AMP Activated Protein Kinase Inhibitor

Examples of AMP activated protein kinase inhibitors include OTSSP-167, JNJ-45261957, ARN-7016, NMS-P635, and APTO-500.

### Programmed Cell Death Inhibitor-1 (PD1)

Examples of programmed cell death inhibitor-1 (PD1) include: avelumab, durvalumab, resminostat, atezolizumab, STI-1014, BMS-936559, MEDI-0680, PSI-001, KY-1003, KD-033, TSR-042.

### Interleukin Ligand 33 Inhibitors

Examples of interleukin ligand 33 inhibitors include AMG-282 and ANB-020.

### PI3K Inhibitors

In some embodiments, the additional therapeutic agent can be a PI3K inhibitor, such as a PI3Kδ inhibitor. PI3K inhibitors include those described in U.S. Publication No. 2004/0266780 and U.S. Publication No. 2008/0275067, incorporated herein by reference in their entirety. PI3K inhibitors useful in the pharmaceutical compositions of the present invention include compounds of Formula (A):

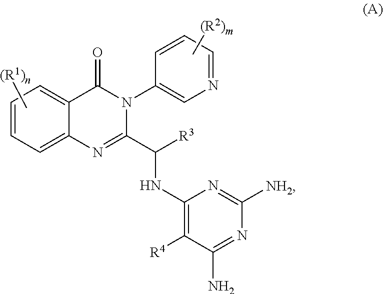

wherein:
n is 0, 1, 2, 3, or 4;
each R¹ is independently halo, cyano, optionally substituted alkyl, optionally substituted cycloalkyl, optionally substituted heterocycloalkyl, optional substituted alkoxy, or SO₂R^{1x} wherein R^{1x} is optionally substituted alkyl;
m is 0, 1, 2, or 3;
each R² is independently halo, —NH₂, optionally substituted alkoxyalkyl, optionally substituted alkyl, optionally substituted cycloalkyl, or optionally substituted heterocycloalkyl;
R³ is hydrogen, optionally substituted alkyl, optionally substituted cycloalkyl, optionally substituted alkoxyalkyl, or optionally substituted heterocycloalkyl; and
R⁴ is selected from halo, cyano, and —CONH₂;
   or a pharmaceutically acceptable salt, tautomer, isomer, a mixture of isomers, or prodrug thereof.

In some embodiments, the at least one additional therapeutic agent is a PI3K inhibitor selected from the group consisting of:

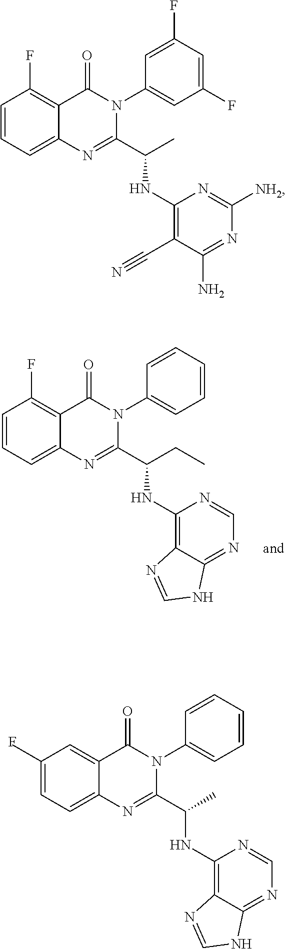

or a pharmaceutically acceptable salt thereof.

### Spleen Tyrosine Kinase (SYK) Inhibitors

Examples of SYK inhibitors include, but are not limited to, 6-(1H-indazol-6-yl)-N-(4-morpholinophenyl)imidazo[1,2-a]pyrazin-8-amine, tamatinib (R406), fostamatinib (R788), PRT062607, BAY-61-3606, NVP-QAB 205 AA, R112, R343, and those described in U.S. Pat. No. 8,450,321 (Gilead Connecticut).

In one embodiment, kits comprising a compound disclosed herein, or a pharmaceutically acceptable salt thereof, in combination with one or more (e.g., one, two, three, one or two, or one to three) additional therapeutic agents are provided.

B. Oncology Agents

The at least one additional therapeutic agent can also be an agent useful for the treatment of cancer and related conditions. In some embodiments, the present invention provides a pharmaceutical composition including a compound of Formula I, at least one additional therapeutic agent that is an oncology agent, and a pharmaceutically acceptable carrier or excipient.

The compounds described herein may be used or combined with a chemotherapeutic agent, an anti-cancer agent, an anti-angiogenic agent, an anti-fibrotic agent, an immunotherapeutic agent, a therapeutic antibody, a radiotherapeutic agent, an anti-neoplastic agent, an anti-proliferation agent, or any combination thereof. These therapeutic agents may be in the forms of compounds, antibodies, polypeptides, or polynucleotides. In some embodiments, the application provides a pharmaceutical composition including a compound of Formula I, a pharmaceutically acceptable carrier or excipient. The pharmaceutical composition can be a combined preparation for simultaneous, separate, or sequential use in therapy, e.g. a method of treating a disease, disorder, or condition that is mediated by PI3K isoforms.

The compound described herein may be used or combined with one or more of the following additional therapeutic agents: an adenosine A2B receptor (A2B) inhibitor, a BET-bromodomain 4 (BRD4) inhibitor, an isocitrate dehydrogenase 1 (IDH1) inhibitor, an IKK inhibitor, a protein kinase C (PKC) activator or inhibitor, a TPL2 inhibitor, a serine/threonine-protein kinase 1 (TBK1) inhibitor, agents that activate or reactivate latent human immunodeficiency virus (HIV) such as panobinostat or romidepsin, an anti-CD20 antibody such as obinutuzumab, an anti-programmed cell death protein 1 (anti-PD-1) antibody such as nivolumab (OPDIVO®, BMS-936558, MDX1106, or MK-34775), durvalumab (MEDI-4736), atezolizumab, and pembrolizumab (KEYTRODA®, MK-3475, SCH-900475, lambrolizumab, CAS Reg. No. 1374853-91-4), and anti-programmed death-ligand 1 (anti-PD-L) antibodies such as BMS-936559, MPDL3280A, MEDI4736, MSB0010718C, and MDX1105-01

The compound disclosed herein and the one or more therapeutic agents (e.g. an A2B inhibitor, an apoptosis signal-regulating kinase (ASK) inhibitor, a Bruton's tyrosine kinase (BTK) inhibitor, a BRD4 inhibitor, a discoidin domain receptor 1 (DDR1) inhibitor, a histone deacetylase (HDAC) inhibitor, an isocitrate dehydrogenase (IDH) inhibitor, a Janus kinase (JAK) inhibitor, a lysyl oxidase-like protein 2 (LOXL2) inhibitor, a matrix metalloprotease 9 (MMP9) inhibitor, a phosphatidylinositol 3-kinase (PI3K) inhibitor, a PKC activator or inhibitor, a spleen tyrosine kinase (SYK) inhibitor, a TPL2 inhibitor, or a TBK inhibitor) may be further used or combined with a chemotherapeutic agent, an anti-cancer agent, an anti-angiogenic agent, an anti-fibrotic agent, an immunotherapeutic agent, a therapeutic antibody, a radiotherapeutic agent, an anti-neoplastic agent, a smoothened (SMO) receptor inhibitor, or any combination thereof.

### Chemotherapeutic Agents

As used herein, the term “chemotherapeutic agent” or “chemotherapeutic” (or “chemotherapy” in the case of treatment with a chemotherapeutic agent) is meant to encompass any non-proteinaceous (i.e., non-peptidic) chemical compound useful in the treatment of cancer.

Chemotherapeutic agents may be categorized by their mechanism of action into, for example, the following groups:
anti-metabolites/anti-cancer agents such as pyrimidine analogs floxuridine, capecitabine, and cytarabine;
purine analogs, folate antagonists (such as pralatrexate), and related inhibitors;
antiproliferative/antimitotic agents including natural products such as vinca alkaloid (vinblastine, vincristine) and microtubule such as taxane (paclitaxel, docetaxel), vinblastin, nocodazole, epothilones, vinorelbine (NAVELBINE®), and epipodophyllotoxins (etoposide, teniposide);
DNA damaging agents such as actinomycin, amsacrine, busulfan, carboplatin, chlorambucil, cisplatin, cyclophosphamide (CYTOXAN®), dactinomycin, daunorubicin, doxorubicin, epirubicin, iphosphamide, melphalan, merchlorethamine, mitomycin, mitoxantrone, nitrosourea, procarbazine, taxol, taxotere, teniposide, etoposide, and triethylenethiophosphoramide;
antibiotics such as dactinomycin, daunorubicin, doxorubicin, idarubicin, anthracyclines, mitoxantrone, bleomycins, plicamycin (mithramycin), and mitomycin;
enzymes such as L-asparaginase which systemically metabolizes L-asparagine and deprives cells which do not have the capacity to synthesize their own asparagine;
antiplatelet agents;
asparaginase stimulators, such as crisantaspase (Erwinase®) and GRASPA (ERY-001, ERY-ASP);
antiproliferative/antimitotic alkylating agents such as nitrogen mustards cyclophosphamide and analogs (melphalan, chlorambucil, hexamethylmelamine, and thiotepa), alkyl nitrosoureas (carmustine) and analogs, streptozocin, and triazenes (dacarbazine);
antiproliferative/antimitotic antimetabolites such as folic acid analogs (methotrexate);
platinum coordination complexes (cisplatin, oxiloplatinim, lobaplatin, and carboplatin), procarbazine, hydroxyurea, mitotane, and aminoglutethimide;
hormones, hormone analogs (estrogen, tamoxifen, goserelin, bicalutamide, and nilutamide), and aromatase inhibitors (letrozole and anastrozole);
anticoagulants such as heparin, synthetic heparin salts, and other inhibitors of thrombin;
fibrinolytic agents such as tissue plasminogen activator, streptokinase, urokinase, aspirin, dipyridamole, ticlopidine, and clopidogrel;
antimigratory agents;
antisecretory agents (breveldin);
immunosuppressives tacrolimus, sirolimus, azathioprine, and mycophenolate;
compounds (TNP-470, genistein) and growth factor inhibitors (vascular endothelial growth factor inhibitors and fibroblast growth factor inhibitors);
angiotensin receptor blockers, nitric oxide donors;
anti-sense oligonucleotides;
antibodies such as trastuzumab and rituximab;
cell cycle inhibitors and differentiation inducers such as tretinoin;
inhibitors, topoisomerase inhibitors (doxorubicin, daunorubicin, dactinomycin, eniposide, epirubicin, etoposide, idarubicin, irinotecan, mitoxantrone, topotecan, sobuzoxane, and irinotecan), and corticosteroids (cortisone, dexamethasone, hydrocortisone, methylprednisolone, prednisone, and prednisolone);
growth factor signal transduction kinase inhibitors;
dysfunction inducers;
toxins such as Cholera toxin, ricin, Pseudomonas exotoxin, Bordetella pertussis adenylate cyclase toxin, diphtheria toxin, and caspase activators;
chromatin;
smoothened (SMO) receptor inhibitors, such as Odomzo® (sonidegib, formerly LDE-225), LEQ506, vismodegib (GDC-0449), BMS-833923, glasdegib (PF-04449913), LY2940680, and itraconazole;
interferon alpha ligand modulators, such as interferon alfa-2b, interferon alpha-2a biosimilar (Biogenomics), ropeginterferon alfa-2b (AOP-2014, P-1101, PEG IFN alpha-2b), Multiferon (Alfanative, Viragen), interferon alpha 1b, Roferon-A (Canferon, Ro-25-3036), interferon alfa-2a follow-on biologic (Biosidus)(Inmutag, Inter 2A), interferon alfa-2b follow-on biologic (Biosidus-Bioferon, Citopheron, Ganapar)(Beijing Kawin Technology-Kaferon)(AXXO-interferon alfa-2b), Alfaferone, pegylated interferon alpha-1b, peginterferon alfa-2b follow-on biologic (Amega), recombinant human interferon alpha-1b, recombinant human interferon alpha-2a, recombinant human interferon alpha-2b, veltuzumab-IFN alpha 2b conjugate, Dynavax (SD-101), and interferon alfa-n1 (Humoferon, SM-10500, Sumiferon);
interferon gamma ligand modulators, such as interferon gamma (OH-6000, Ogamma 100);
Complement C3 modulators, such as Imprime PGG;
IL-6 receptor modulators, such as tocilizumab, siltuximab, AS-101 (CB-06-02, IVX-Q-101);
Telomerase modulators, such as tertomotide (GV-1001, HR-2802, Riavax) and imetelstat (GRN-163, JNJ-63935937);
DNA methyltransferases inhibitors, such as temozolomide (CCRG-81045), decitabine, guadecitabine (S-110, SGI-110), KRX-0402, and azacitidine;
DNA gyrase inhibitors, such as pixantrone and sobuzoxane;
Bcl-2 family protein inhibitor ABT-263, venetoclax (ABT-199), ABT-737, and AT-101.

Further examples of chemotherapeutic agents include:
alkylating agents such as thiotepa and cyclophosphamide (CYTOXAN®);
alkyl sulfonates such as busulfan, improsulfan, and piposulfan;
aziridines such as benzodepa, carboquone, meturedepa, and uredepa;
ethylenimines and methylamelamines including altretamine, triethylenemelamine, triethylenephosphoramide, triethylenethiophosphoramide, and trimemylolomelamine;
acetogenins, especially bullatacin and bullatacinone;
a camptothecin, including synthetic analog topotecan;
bryostatin;
callystatin;
CC-1065, including its adozelesin, carzelesin, and bizelesin synthetic analogs;
cryptophycins, particularly cryptophycin 1 and cryptophycin 8;
dolastatin;
duocarmycin, including the synthetic analogs KW-2189 and CBI-TMI;
eleutherobin;
pancratistatin;
a sarcodictyin;
spongistatin;
nitrogen mustards such as chlorambucil, chlornaphazine, cyclophosphamide, estramustine, ifosfamide, mechlorethamine, mechlorethamine oxide hydrochloride, melphalan, novembichin, phenesterine, prednimustine, trofosfamide, and uracil mustard;
nitrosoureas such as carmustine, chlorozotocin, foremustine, lomustine, nimustine, and ranimustine;
antibiotics such as the enediyne antibiotics (e.g., calicheamicin, especially calicheamicin gammaII and calicheamicin phiI1), dynemicin including dynemicin A, bisphosphonates such as clodronate, an esperamicin, neocarzinostatin chromophore and related chromoprotein enediyne antibiotic chromomophores, aclacinomycins, actinomycin, authramycin, azaserine, bleomycins, cactinomycin, carabicin, carrninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, and zorubicin;
anti-metabolites such as methotrexate and 5-fluorouracil (5-FU);
folic acid analogs such as demopterin, methotrexate, pteropterin, and trimetrexate;
purine analogs such as fludarabine, 6-mercaptopurine, thiamiprine, and thioguanine;
pyrimidine analogs such as ancitabine, azacitidine, 6-azauridine, carmofur, cytarabine, dideoxyuridine, doxifluridine, enocitabine, and floxuridine;
androgens such as calusterone, dromostanolone propionate, epitiostanol, mepitiostane, and testolactone;
anti-adrenals such as aminoglutethimide, mitotane, and trilostane;
folic acid replinishers such as frolinic acid;
trichothecenes, especially T-2 toxin, verracurin A, roridin A, and anguidine;
taxoids such as paclitaxel (TAXOL®) and docetaxel (TAXOTERE®);
platinum analogs such as cisplatin and carboplatin;
aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; pentostatin; phenamet; pirarubicin; losoxantrone; fluoropyrimidine; folinic acid; podophyllinic acid; 2-ethylhydrazide; procarbazine; polysaccharide-K (PSK); razoxane; rhizoxin; sizofiran; spirogermanium; tenuazonic acid; triaziquone; 2,2′,2″-tricUorotriemylamine; urethane; vindesine; dacarbazine; mannomustine; mitobronitol; mitolactol; pipobroman; gacytosine; arabinoside (“Ara-C”); cyclophosphamide; thiopeta; chlorambucil; gemcitabine (GEMZAR®); 6-thioguanine; mercaptopurine; methotrexate; vinblastine; platinum; etoposide (VP-16); ifosfamide; mitroxantrone; vancristine; vinorelbine (NAVELBINE®); novantrone; teniposide; edatrexate; daunomycin; aminopterin; xeoloda; ibandronate; CPT-11; topoisomerase inhibitor RFS 2000; difluoromethylornithine (DFMO); retinoids such as retinoic acid; capecitabine; FOLFIRI (fluorouracil, leucovorin, and irinotecan);
and pharmaceutically acceptable salts, acids, or derivatives of any of the above.

### Anti-Hormonal Agents

Also included in the definition of “chemotherapeutic agent” are anti-hormonal agents such as anti-estrogens and selective estrogen receptor modulators (SERMs), inhibitors of the enzyme aromatase, anti-androgens, and pharmaceutically acceptable salts, acids or derivatives of any of the above that act to regulate or inhibit hormone action on tumors.

Examples of anti-estrogens and SERMs include, for example, tamoxifen (including NOLVADEX™), raloxifene, droloxifene, 4-hydroxytamoxifen, trioxifene, keoxifene, LY117018, onapristone, and toremifene (FARESTON®).

Inhibitors of the enzyme aromatase regulate estrogen production in the adrenal glands. Examples include 4(5)-imidazoles, aminoglutethimide, megestrol acetate (MEGACE®), exemestane, formestane, fadrozole, vorozole (RIVISOR®), letrozole (FEMARA®), and anastrozole (ARIMIDEX®).

Examples of anti-androgens include flutamide, nilutamide, bicalutamide, leuprohde, and goserelin.

### Anti-Angiogenic Agents

Anti-angiogenic agents include, but are not limited to, retinoid acid and derivatives thereof, 2-methoxyestradiol, ANGIOSTATIN®, ENDOSTATIN®, suramin, squalamine, tissue inhibitor of metalloproteinase-1, tissue inhibitor of metalloproteinase-2, plasminogen activator inhibitor-1, plasminogen activator inbibitor-2, cartilage-derived inhibitor, paclitaxel (nab-paclitaxel), platelet factor 4, protamine sulphate (clupeine), sulphated chitin derivatives (prepared from queen crab shells), sulphated polysaccharide peptidoglycan complex (sp-pg), staurosporine, modulators of matrix metabolism including proline analogs such as 1-azetidine-2-carboxylic acid (LACA), cishydroxyproline, d,I-3,4-dehydroproline, thiaproline, α,α′-dipyridyl, beta-aminopropionitrile fumarate, 4-propyl-5-(4-pyridinyl)-2(3h)-oxazolone, methotrexate, mitoxantrone, heparin, interferons, interferon alpha ligand modulators, 2 macroglobulin-serum, chicken inhibitor of metalloproteinase-3 (ChIMP-3), chymostatin, beta-cyclodextrin tetradecasulfate, eponemycin, fumagillin, gold sodium thiomalate, d-penicillamine, beta-1-anticollagenase-serum, alpha-2-antiplasmin, bisantrene, lobenzarit disodium, n-2-carboxyphenyl-4-chloroanthronilic acid disodium or “CCA”, thalidomide, angiostatic steroid, carboxy aminoimidazole, and metalloproteinase inhibitors such as BB-94. Other anti-angiogenesis agents include antibodies, preferably monoclonal antibodies against these angiogenic growth factors: beta-FGF, alpha-FGF, FGF-5, VEGF isoforms, VEGF-C, HGF/SF, and Ang-1/Ang-2.

### Anti-Fibrotic Agents

Anti-fibrotic agents include, but are not limited to, the compounds such as beta-aminoproprionitrile (BAPN), as well as the compounds disclosed in U.S. Pat. No. 4,965,288 relating to inhibitors of lysyl oxidase and their use in the treatment of diseases and conditions associated with the abnormal deposition of collagen and U.S. Pat. No. 4,997,854 relating to compounds which inhibit LOX for the treatment of various pathological fibrotic states, which are herein incorporated by reference. Further exemplary inhibitors are described in U.S. Pat. No. 4,943,593 relating to compounds such as 2-isobutyl-3-fluoro-, chloro-, or bromo-allylamine, U.S. Pat. No. 5,021,456, U.S. Pat. No. 5,059,714, U.S. Pat. No. 5,120,764, U.S. Pat. No. 5,182,297, U.S. Pat. No. 5,252,608 relating to 2-(1-naphthyloxymemyl)-3-fluoroallylamine, and US 2004-0248871, which are herein incorporated by reference.

Exemplary anti-fibrotic agents also include the primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives; semicarbazide and urea derivatives; aminonitriles such as BAPN or 2-nitroethylamine; unsaturated or saturated haloamines such as 2-bromo-ethylamine, 2-chloroethylamine, 2-trifluoroethylamine, 3-bromopropylamine, and p-halobenzylamines; and selenohomocysteine lactone.

Other anti-fibrotic agents are copper chelating agents penetrating or not penetrating the cells. Exemplary compounds include indirect inhibitors which block the aldehyde derivatives originating from the oxidative deamination of the lysyl and hydroxylysyl residues by the lysyl oxidases. Examples include the thiolamines, particularly D-penicillamine, and its analogs such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphurate, 2-acetamidoethyl-2-acetamidoethanethiol sulphanate, and sodium-4-mercaptobutanesulphinate trihydrate.

### Immunotherapeutic Agents

The immunotherapeutic agents include and are not limited to therapeutic antibodies suitable for treating patients. Some examples of therapeutic antibodies include simtuzumab, abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab (YERVOY®, MDX-010, BMS-734016, and MDX-101), iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nofetumomab, obinutuzumab, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, and 3F8. Rituximab can be used for treating indolent B-cell cancers, including marginal-zone lymphoma, WM, CLL and small lymphocytic lymphoma. A combination of Rituximab and chemotherapy agents is especially effective.

The exemplified therapeutic antibodies may be further labeled or combined with a radioisotope particle such as indium-111, yttrium-90, or iodine-131.

### Lymphoma or Leukemia Combination Therapy

Some chemotherapy agents are suitable for treating lymphoma or leukemia. These agents include aldesleukin, alvocidib, antineoplaston AS2-1, antineoplaston A10, anti-thymocyte globulin, amifostine trihydrate, aminocamptothecin, arsenic trioxide, beta alethine, Bcl-2 family protein inhibitor ABT-263, ABT-199, BMS-345541, bortezomib (VELCADE®), carfilzomib (Kyprolis®), vemurafenib (Zelboraf®), Omr-IgG-am (WNIG, Omrix), bryostatin 1, busulfan, carboplatin, campath-1H, CC-5103, carmustine, caspofungin acetate, clofarabine, cisplatin, cladribine, chlorambucil, curcumin, cyclosporine, cyclophosphamide, cytarabine, denileukin diftitox, dexamethasone, DT-PACE (dexamethasone, thalidomide, cisplatin, doxorubicin, cyclophosphamide, and etoposide), docetaxel, dolastatin 10, doxorubicin, doxorubicin hydrochloride, enzastaurin, epoetin alfa, etoposide, everolimus (RAD001), fenretinide, filgrastim, melphalan, mesna, flavopiridol, fludarabine, geldanamycin (17-AAG), ifosfamide, irinotecan hydrochloride, ixabepilone, lenalidomide (REVLIMID®, CC-5013), lymphokine-activated killer cells, melphalan, methotrexate, mitoxantrone hydrochloride, motexafin gadolinium, mycophenolate mofetil, nelarabine, oblimersen, obatoclax (GX15-070), oblimersen, octreotide acetate, omega-3 fatty acids, oxaliplatin, paclitaxel, PD0332991, PEGylated liposomal doxorubicin hydrochloride, pegfilgrastim, pentostatin, perifosine, prednisolone, prednisone, R-roscovitine (seliciclib, CYC202), recombinant interferon alfa, recombinant interleukin-12, recombinant interleukin-11, recombinant flt3 ligand, recombinant human thrombopoietin, rituximab, sargramostim, sildenafil citrate, simvastatin, sirolimus, styryl sulphones, tacrolimus, tanespimycin, temsirolimus (CCl-779), thalidomide, therapeutic allogeneic lymphocytes, thiotepa, tipifarnib, vincristine, vincristine sulfate, vinorelbine ditartrate, SAHA (suberanilohydroxamic acid, or suberoyl, anilide, and hydroxamic acid), FR (fludarabine and rituximab), CHOP (cyclophosphamide, doxorubicin, vincristine, and prednisone), CVP (cyclophosphamide, vincristine, and prednisone), FCM (fludarabine, cyclophosphamide, and mitoxantrone), FCR (fludarabine, cyclophosphamide, and rituximab), hyperCVAD (hyperfractionated cyclophosphamide, vincristine, doxorubicin, dexamethasone, methotrexate, and cytarabine), ICE (iphosphamide, carboplatin, and etoposide), MCP (mitoxantrone, chlorambucil, and prednisolone), R-CHOP (rituximab and CHOP), R-CVP (rituximab and CVP), R-FCM (rituximab and FCM), R-ICE (rituximab and ICE), and R-MCP (rituximab and MCP).

One modified approach is radioimmunotherapy, wherein a monoclonal antibody is combined with a radioisotope particle, such as indium-111, yttrium-90, and iodine-131. Examples of combination therapies include, but are not limited to, iodine-131 tositumomab (BEXXAR®), yttrium-90 ibritumomab tiuxetan (ZEVALIN®), and BEXXAR® with CHOP.

The abovementioned therapies can be supplemented or combined with stem cell transplantation or treatment. Therapeutic procedures include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme technique, low-LET cobalt-60 gamma ray therapy, bleomycin, conventional surgery, radiation therapy, and nonmyeloablative allogeneic hematopoietic stem cell transplantation.

### Non-Hodgkin's Lymphomas Combination Therapy

Treatment of non-Hodgkin's lymphomas (NHL), especially those of B cell origin, includes using monoclonal antibodies, standard chemotherapy approaches (e.g., CHOP, CVP, FCM, MCP, and the like), radioimmunotherapy, and combinations thereof, especially integration of an antibody therapy with chemotherapy.

Examples of unconjugated monoclonal antibodies for the treatment of NHL/B-cell cancers include rituximab, alemtuzumab, human or humanized anti-CD20 antibodies, lumiliximab, anti-TNF-related apoptosis-inducing ligand (anti-TRAIL), bevacizumab, galiximab, epratuzumab, SGN-40, and anti-CD74.

Examples of experimental antibody agents used in treatment of NHL/B-cell cancers include ofatumumab, ha20, PRO131921, alemtuzumab, galiximab, SGN-40, CHIR-12.12, epratuzumab, lumiliximab, apolizumab, milatuzumab, and bevacizumab.

Examples of standard regimens of chemotherapy for NHL/B-cell cancers include CHOP, FCM, CVP, MCP, R-CHOP, R-FCM, R-CVP, and R-MCP.

Examples of radioimmunotherapy for NHL/B-cell cancers include yttrium-90 ibritumomab tiuxetan (ZEVALIN®) and iodine-131 tositumomab (BEXXAR®).

### Mantle Cell Lymphoma Combination Therapy

Therapeutic treatments for mantle cell lymphoma (MCL) include combination chemotherapies such as CHOP, hyperCVAD, and FCM. These regimens can also be supplemented with the monoclonal antibody rituximab to form combination therapies R-CHOP, hyperCVAD-R, and R-FCM. Any of the abovementioned therapies may be combined with stem cell transplantation or ICE in order to treat MCL.

An alternative approach to treating MCL is immunotherapy. One immunotherapy uses monoclonal antibodies like rituximab. Another uses cancer vaccines, such as GTOP-99, which are based on the genetic makeup of an individual patient's tumor.

A modified approach to treat MCL is radioimmunotherapy, wherein a monoclonal antibody is combined with a radioisotope particle, such as iodine-131 tositumomab (BEXXAR®) and yttrium-90 ibritumomab tiuxetan (ZEVALIN®). In another example, BEXXAR® is used in sequential treatment with CHOP.

Other approaches to treating MCL include autologous stem cell transplantation coupled with high-dose chemotherapy, administering proteasome inhibitors such as bortezomib (VELCADE® or PS-341), or administering antiangiogenesis agents such as thalidomide, especially in combination with rituximab.

Another treatment approach is administering drugs that lead to the degradation of Bcl-2 protein and increase cancer cell sensitivity to chemotherapy, such as oblimersen, in combination with other chemotherapeutic agents.

A further treatment approach includes administering mTOR inhibitors, which can lead to inhibition of cell growth and even cell death. Non-limiting examples are sirolimus, temsirolimus (TORISEL®, CCI-779), CC-115, CC-223, SF-1126, PQR-309, voxtalisib, GSK-2126458, and temsirolimus in combination with RITUXAN®, VELCADE®, or other chemotherapeutic agents.

Other recent therapies for MCL have been disclosed. Such examples include flavopiridol, PD0332991, R-roscovitine (selicicilib, CYC202), styryl sulphones, obatoclax (GX15-070), TRAIL, Anti-TRAIL death receptors DR4 and DR5 antibodies, temsirolimus (TORISEL®, CCl-779), everolimus (RAD001), BMS-345541, curcumin, SAHA, thalidomide, lenalidomide (REVLIMID®, CC-5013), and geldanamycin (17-AAG).

### Waldenstrom's Macroglobulinemia Combination Therapy

Therapeutic agents used to treat Waldenstrom's Macroglobulinemia (WM) include perifosine, bortezomib (VELCADE®), rituximab, sildenafil citrate (VIAGRA®), CC-5103, thalidomide, epratuzumab (hLL2-anti-CD22 humanized antibody), simvastatin, enzastaurin, campath-1H, dexamethasone, DT-PACE, oblimersen, antineoplaston A10, antineoplaston AS2-1, alemtuzumab, beta alethine, cyclophosphamide, doxorubicin hydrochloride, prednisone, vincristine sulfate, fludarabine, filgrastim, melphalan, recombinant interferon alfa, carmustine, cisplatin, cyclophosphamide, cytarabine, etoposide, melphalan, dolastatin 10, indium-111 monoclonal antibody MN-14, yttrium-90 humanized epratuzumab, anti-thymocyte globulin, busulfan, cyclosporine, methotrexate, mycophenolate mofetil, therapeutic allogeneic lymphocytes, yttrium-90 ibritumomab tiuxetan, sirolimus, tacrolimus, carboplatin, thiotepa, paclitaxel, aldesleukin, docetaxel, ifosfamide, mesna, recombinant interleukin-11, recombinant interleukin-12, Bcl-2 family protein inhibitor ABT-263, denileukin diftitox, tanespimycin, everolimus, pegfilgrastim, vorinostat, alvocidib, recombinant flt3 ligand, recombinant human thrombopoietin, lymphokine-activated killer cells, amifostine trihydrate, aminocamptothecin, irinotecan hydrochloride, caspofungin acetate, clofarabine, epoetin alfa, nelarabine, pentostatin, sargramostim, vinorelbine ditartrate, WT-1 analog peptide vaccine, WT1 126-134 peptide vaccine, fenretinide, ixabepilone, oxaliplatin, monoclonal antibody CD19 (such as tisagenlecleucel-T, CART-19, CTL-019), monoclonal antibody CD20, omega-3 fatty acids, mitoxantrone hydrochloride, octreotide acetate, tositumomab, iodine-131 tositumomab, motexafin gadolinium, arsenic trioxide, tipifarnib, autologous human tumor-derived HSPPC-96, veltuzumab, bryostatin 1, PEGylated liposomal doxorubicin hydrochloride, and any combination thereof.

Examples of therapeutic procedures used to treat WM include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme techniques, low-LET cobalt-60 gamma ray therapy, bleomycin, conventional surgery, radiation therapy, and nonmyeloablative allogeneic hematopoietic stem cell transplantation.

### Diffuse Large B-Cell Lymphoma Combination Therapy

Therapeutic agents used to treat diffuse large B-cell lymphoma (DLBCL) include cyclophosphamide, doxorubicin, vincristine, prednisone, anti-CD20 monoclonal antibodies, etoposide, bleomycin, many of the agents listed for WM, and any combination thereof, such as ICE and R-ICE.

### Chronic Lymphocytic Leukemia Combination Therapy

Examples of therapeutic agents used to treat chronic lymphocytic leukemia (CLL) include chlorambucil, cyclophosphamide, fludarabine, pentostatin, cladribine, doxorubicin, vincristine, prednisone, prednisolone, alemtuzumab, many of the agents listed for WM, and combination chemotherapy and chemoimmunotherapy, including the following common combination regimens: CVP, R-CVP, ICE, R-ICE, FCR, and FR.

### Myelofibrosis Combination Therapy

Myelofibrosis inhibiting agents include, but are not limited to, hedgehog inhibitors, histone deacetylase (HDAC) inhibitors, and tyrosine kinase inhibitors. A non-limiting example of hedgehog inhibitors is saridegib.

Examples of HDAC inhibitors include, but are not limited to, pracinostat and panobinostat.

Non-limiting examples of tyrosine kinase inhibitors are lestaurtinib, bosutinib, imatinib, gilteritinib, radotinib, and cabozantinib.

### Hyperproliferative Disorder Combination Therapy

Gemcitabine, nab-paclitaxel, and gemcitabine/nab-paclitaxel may be used with a JAK inhibitor and/or PI3Kδ inhibitor to treat hyperproliferative disorders.

### Kinase Inhibitors

In one embodiment, the compound described herein may be used or combined with one or more additional therapeutic agents. The one or more therapeutic agents include, but are not limited to, an inhibitor of Abl, activated CDC kinase (ACK) such as ACK1, adenosine A2B receptor (A2B), apoptosis signal-regulating kinase (ASK), Aurora kinase, Bruton's tyrosine kinase (BTK), BET-bromodomain (BRD) such as BRD4, c-Kit, c-Met, CDK-activating kinase (CAK), calmodulin-dependent protein kinase (CaMK), cyclin-dependent kinase (CDK), casein kinase (CK), discoidin domain receptor (DDR), epidermal growth factor receptors (EGFR), focal adhesion kinase (FAK), Flt-3, farnesoid x receptor (FXR), FYN, glycogen synthase kinase (GSK), HCK, histone deacetylase (HDAC), indoleamine 2,3-dioxygenase (IDO), I-Kappa-B kinase (IKK) such as IKKβε, isocitrate dehydrogenase (IDH) such as IDH1, Janus kinase (JAK), KDR, lysine demethylase (KDM5), lymphocyte-specific protein tyrosine kinase (LCK), lysyl oxidase protein (LOX), lysyl oxidase-like protein (LOXL), LYN, matrix metalloprotease (MMP), mitogen-activated protein kinase (MEK), mitogen-activated protein kinase (MAPK), mut T homolog (MTH), NEK9, NPM-ALK, p38 kinase, platelet-derived growth factor (PDGF), phosphorylase kinase (PK), polo-like kinase (PLK), phosphatidylinositol 3-kinase (PI3K), protein kinase (PK) such as protein kinase A, B, and/or C, PYK, spleen tyrosine kinase (SYK), serine/threonine kinase TPL2, serine/threonine kinase (STK), signal transduction and transcription (STAT), SRC, serine/threonine-protein kinase (TBK) such as TBK1, TIE, tyrosine kinase (TK), tank-binding kinase (TBK), vascular endothelial growth factor receptor (VEGFR), YES, or any combination thereof.

### Apoptosis Signal-Regulating Kinase (ASK) Inhibitors

ASK inhibitors include ASK1 inhibitors. Examples of ASK1 inhibitors include, but are not limited to, those described in WO 2011/008709 (Gilead Sciences) and WO 2013/112741 (Gilead Sciences).

### Bruton's Tyrosine Kinase (BTK) Inhibitors

Examples of BTK inhibitors include, but are not limited to, (S)-6-amino-9-(1-(but-2-ynoyl)pyrrolidin-3-yl)-7-(4-phenoxyphenyl)-7H-purin-8(9H)-one, ibrutinib, HM71224, ONO-4059, and CC-292.

### Mitogen-Activated Protein Kinase (MEK) Inhibitors

MEK inhibitors include selumetinib (AZD6244), MT-144, sorafenib, trametinib (GSK1120212), binimetinib, antroquinonol, uprosertib+trametinib,

### Casein Kinase (CK) Inhibitors

CK inhibitors include CK1 and/or CK2.

### Cyclin-Dependent Kinase (CDK) Inhibitors

CDK inhibitors include inhibitors of CDK 1, 2, 3, 4, and/or 6. Examples of CDK inhibitors include rigosertib, selinexor, UCN-01, alvocidib (HMR-1275, flavopiridol), FLX-925, AT-7519, abemaciclib, palbociclib, and TG-02.

### Discoidin Domain Receptor (DDR) Inhibitors

DDR inhibitors include inhibitors of DDR1 and/or DDR2. Examples of DDR inhibitors include, but are not limited to, those disclosed in WO 2014/047624 (Gilead Sciences), US 2009-0142345 (Takeda Pharmaceutical), US 2011-0287011 (Oncomed Pharmaceuticals), WO 2013/027802 (Chugai Pharmaceutical), and WO 2013/034933 (Imperial Innovations).

### Histone Deacetylase (HDAC) Inhibitors

Examples of HDAC inhibitors include, but are not limited to, pracinostat, CS-055 (HBI-8000), resminostat, entinostat, abexinostat, belinostat, vorinostat, riclinostat, CUDC-907, ACY-241, CKD-581, SHP-141, valproic acid (VAL-001), givinostat, quisinostat (JNJ-26481585), BEBT-908 and panobinostat.

### Janus Kinase (JAK) Inhibitors

JAK inhibitors inhibit JAK1, JAK2, and/or JAK3, and/or Tyk 2. Examples of JAK inhibitors include, but are not limited to, Compound A, momelotinib (CYT0387), ruxolitinib, filgotinib (GLPG0634), peficitinib (ASP015K), fedratinib, tofacitinib (formerly tasocitinib), baricitinib, lestaurtinib, pacritinib (SB1518), XL019, AZD1480, INCB039110, LY2784544, BMS911543, AT9283, and NS018.

### Lysyl Oxidase-Like Protein (LOXL) Inhibitors

LOXL inhibitors include inhibitors of LOXL1, LOXL2, LOXL3, LOXL4, and/or LOXL5. Examples of LOXL inhibitors include, but are not limited to, the antibodies described in WO 2009/017833 (Arresto Biosciences).

Examples of LOXL2 inhibitors include, but are not limited to, the antibodies described in WO 2009/017833 (Arresto Biosciences), WO 2009/035791 (Arresto Biosciences), and WO 2011/097513 (Gilead Biologics).

In certain embodiments, the LOXL2 inhibitor is an anti-LOXL2 antibody (see, e.g., U.S. Pat. No. 8,461,303, and U.S. Publication Nos. 2012/0309020, 2013/0324705, and 2014/0079707, each of which are incorporated herein by reference in their entirety). The anti-LOXL2 antibody can be a monoclonal antibody (including full length monoclonal antibody), polyclonal antibody, human antibody, humanized antibody, chimeric antibody, diabody, multispecific antibody (e.g., bispecific antibody), or an antibody fragment including, but not limited to, a single chain binding polypeptide, so long as it exhibits the desired biological activity. Exemplified anti-LOXL2 antibody or antigen binding fragment thereof may be found in U.S. Publication Nos. 2012/0309020, 2013/0324705, 2014/0079707, 2009/0104201, 2009/0053224, and 2011/0200606, each of which is incorporated herein by reference in the entirety).

### Matrix Metalloprotease (MMP) Inhibitors

MMP inhibitors include inhibitors of MMP1 through 10. Examples of MMP9 inhibitors include, but are not limited to, marimastat (BB-2516), cipemastat (Ro 32-3555), and those described in WO 2012/027721 (Gilead Biologics).

### Polo-Like Kinase (PLK) Inhibitors

PLK inhibitors include inhibitors of PLK 1, 2, and 3.

Phosphatidylinositol 3-kinase (PI3K) Inhibitors

PI3K inhibitors include inhibitors of PI3Kγ, PI3Kδ, PI3Kβ, PI3Kα, and/or pan-PI3K. Examples of PI3K inhibitors include, but are not limited to, wortmannin, BKM120, CH5132799, XL756, idelalisib (Zydelig®), and GDC-0980.

Examples of PI3Kγ inhibitors include, but are not limited to, ZSTK474, AS252424, LY294002, and TG100115.

Examples of PI3Kδ inhibitors include, but are not limited to, Compound B, Compound C, Compound D, Compound E, PI3K II, TGR-1202, AMG-319, GSK2269557, X-339, X-414, RP5090, KAR4141, XL499, OXY111A, IPI-145, IPI-443, and the compounds described in WO 2005/113556 (ICOS), WO 2013/052699 (Gilead Calistoga), WO 2013/116562 (Gilead Calistoga), WO 2014/100765 (Gilead Calistoga), WO 2014/100767 (Gilead Calistoga), and WO 2014/201409 (Gilead Sciences).

Examples of PI3Kβ inhibitors include, but are not limited to, GSK2636771, BAY 10824391, and TGX221.

Examples of PI3Kα inhibitors include, but are not limited to, buparlisib, BAY 80-6946, BYL719, PX-866, RG7604, MLN1117, WX-037, AEZA-129, and PA799.

Examples of pan-PI3K inhibitors include, but are not limited to, LY294002, BEZ235, XL147 (SAR245408), and GDC-0941.

### Spleen Tyrosine Kinase (SYK) Inhibitors

Examples of SYK inhibitors include, but are not limited to, 6-(1H-indazol-6-yl)-N-(4-morpholinophenyl)imidazo[1,2-a]pyrazin-8-amine, tamatinib (R406), fostamatinib (R788), PRT062607, BAY-61-3606, NVP-QAB 205 AA, R112, R343, and those described in U.S. Pat. No. 8,450,321 (Gilead Connecticut), and those described in U.S. Publication No. 2015/0175616, which is incorporated by reference herein in its entirety.

### Tyrosine-Kinase Inhibitors (TKIs)

TKIs may target epidermal growth factor receptors (EGFRs) and receptors for fibroblast growth factor (FGF), platelet-derived growth factor (PDGF), and vascular endothelial growth factor (VEGF). Examples of TKIs that target EGFR include, but are not limited to, gefitinib, nintedanib, and erlotinib. Sunitinib is a non-limiting example of a TKI that targets receptors for FGF, PDGF, and VEGF. Additional TKIs include dasatinib and ponatinib.

### Toll-Like Receptor (TLR) Modulators

TLR modulators include inhibitors of TLR-1, TLR-2, TLR-3, TLR-4, TLR-5, TLR-6, TLR-7, TLR-8, TLR-9, TLR-10, TLR-11, TLR-12, and/or TLR-13.

C. ASK-1 Inhibitors

The at least one additional therapeutic agent can also be an apoptosis signal-regulating kinase 1 (ASK-1) inhibitor. In some embodiments, the present invention provides a pharmaceutical composition including a compound of Formula I, at least one additional therapeutic agent that is an apoptosis signal-regulating kinase 1 (ASK-1) inhibitor, and a pharmaceutically acceptable carrier or excipient.

U.S. Publication No. 2001/00095410, published Jan. 13, 2011, which is incorporated in its entirety herein, discloses compounds useful as ASK-1 inhibitors. U.S. Publication 2001/00095410, incorporated in its entirety herein, relates to compounds of Formula (C) and/or Formula (D):

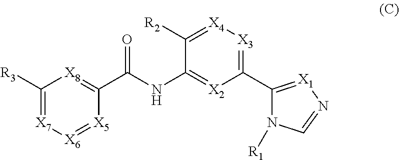

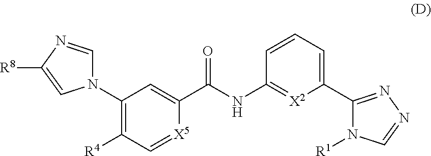

or a pharmaceutically acceptable salt thereof, wherein each of the variables (e.g. X₁-X₈ and R₁-R₃ and R₈) are as defined therein.

Additional examples of ASK1 inhibitors may be found in U.S. Pat. No. 8,440,665, which is incorporated herein by reference in its entirety. U.S. Pat. No. 8,440,665 describes, among other things, compounds of formula (E):

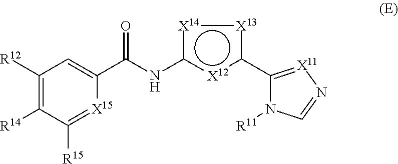

wherein the variables X¹²-X¹⁵ and R¹¹-R¹⁵ are as described therein.

Additional exemplary ASK1 inhibitors, the methods of preparation thereof, or the methods of use thereof may be found in U.S. Publication nos. 2011/0009410 and 2013/0197037, each of which is incorporated herein by reference in the entirety.

In some embodiments, the ASK-1 inhibitor can be

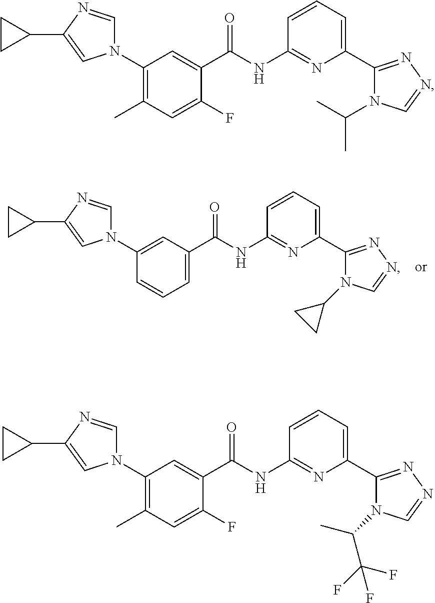 [SMILES: Cc1cc(F)c(C(=O)Nc2cccc(-c3nncn3C(C)C)n2)cc1-n1cnc(C2CC2)c1 ; O=C(Nc1cccc(-c2nncn2C2CC2)n1)c1cccc(-n2cnc(C3CC3)c2)c1 ; Cc1cc(F)c(C(=O)Nc2cccc(-c3nncn3[C@@H](C)C(F)(F)F)n2)cc1-n1cnc(C2CC2)c1]

or a pharmaceutically acceptable salt thereof.

D. Cardiovascular Agents

The at least one additional therapeutic agent can also be a cardiovascular agent. In some embodiments, the present invention provides a pharmaceutical composition including a compound of Formula I, at least one additional therapeutic agent that is a cardiovascular agent, and a pharmaceutically acceptable carrier or excipient. In some embodiments, the at least one additional therapeutic agent is selected from the group consisting of anti-anginals, heart failure agents, antithrombotic agents, antiarrhythmic agents, antihypertensive agents, and lipid lowering agents.

Patients being treated by administration of the late sodium channel blockers of the disclosure often exhibit diseases or conditions that benefit from treatment with other therapeutic agents. These diseases or conditions can be of the cardiovascular nature or can be related to pulmonary disorders, metabolic disorders, gastrointestinal disorders and the like. Additionally, some coronary patients being treated by administration of the late sodium channel blockers of the disclosure exhibit conditions that can benefit from treatment with therapeutic agents that are antibiotics, analgesics, and/or antidepressants and anti-anxiety agents.

### Cardiovascular Agent Combination Therapy

Cardiovascular related diseases or conditions that can benefit from a combination treatment of the late sodium channel blockers of the disclosure with other therapeutic agents include, without limitation, angina including stable angina, unstable angina (UA), exercised-induced angina, variant angina, arrhythmias, intermittent claudication, myocardial infarction including non-STE myocardial infarction (NSTEMI), pulmonary hypertension including pulmonary arterial hypertension, heart failure including congestive (or chronic) heart failure and diastolic heart failure and heart failure with preserved ejection fraction (diastolic dysfunction), acute heart failure, or recurrent ischemia.

Therapeutic agents suitable for treating cardiovascular related diseases or conditions include anti-anginals, heart failure agents, antithrombotic agents, antiarrhythmic agents, antihypertensive agents, and lipid lowering agents.

The co-administration of the late sodium channel blockers of the disclosure with therapeutic agents suitable for treating cardiovascular related conditions allows enhancement in the standard of care therapy the patient is currently receiving.

### Anti-Anginals

Anti-anginals include beta-blockers, calcium channel blockers, and nitrates. Beta blockers reduce the heart's need for oxygen by reducing its workload resulting in a decreased heart rate and less vigorous heart contraction. Examples of beta-blockers include acebutolol (Sectral®), atenolol (Tenormin®), betaxolol (Kerlone®), bisoprolol/hydrochlorothiazide (Ziac®), bisoprolol (Zebeta®), carteolol (Cartrol®), esmolol (Brevibloc®), labetalol (Normodyne®, Trandate®), metoprolol (Lopressor®, Toprol® XL), nadolol (Corgard®), propranolol (Inderal®), sotalol (Betapace®), and timolol (Blocadren®). In some embodiments, the at least one additional therapeutic agent can be an anti-anginal selected from beta-blockers, calcium channel blockers, and nitrates.

Nitrates dilate the arteries and veins thereby increasing coronary blood flow and decreasing blood pressure. Examples of nitrates include nitroglycerin, nitrate patches, isosorbide dinitrate, and isosorbide-5-mononitrate.

Calcium channel blockers prevent the normal flow of calcium into the cells of the heart and blood vessels causing the blood vessels to relax thereby increasing the supply of blood and oxygen to the heart. Examples of calcium channel blockers include amlodipine (Norvasc®, Lotrel®), bepridil (Vascor®), diltiazem (Cardizem®, Tiazac®), felodipine (Plendil®), nifedipine (Adalat®, Procardia®), nimodipine (Nimotop®), nisoldipine (Sular®), verapamil (Calan®, Isoptin®, Verelan®), and nicardipine.

### Heart Failure Agents

Agents used to treat heart failure include diuretics, ACE inhibitors, vasodilators, and cardiac glycosides. Diuretics eliminate excess fluids in the tissues and circulation thereby relieving many of the symptoms of heart failure. Examples of diuretics include hydrochlorothiazide, metolazone (Zaroxolyn®), furosemide (Lasix®), bumetanide (Bumex®), spironolactone (Aldactone®), and eplerenone (Inspra®). In some embodiments, the at least one additional therapeutic agent can be a heart failure agent selected from diuretics, ACE inhibitors, vasodilators, and cardiac glycosides.

Angiotensin converting enzyme (ACE) inhibitors reduce the workload on the heart by expanding the blood vessels and decreasing resistance to blood flow. Examples of ACE inhibitors include benazepril (Lotensin®), captopril (Capoten®), enalapril (Vasotec®), fosinopril (Monopril®), lisinopril (Prinivil®, Zestril®), moexipril (Univasc®), perindopril (Aceon®), quinapril (Accupril®), ramipril (Altace®), and trandolapril (Mavik®).

Vasodilators reduce pressure on the blood vessels by making them relax and expand. Examples of vasodilators include hydralazine, diazoxide, prazosin, clonidine, and methyldopa. ACE inhibitors, nitrates, potassium channel activators, and calcium channel blockers also act as vasodilators.

Cardiac glycosides are compounds that increase the force of the heart's contractions.

These compounds strengthen the pumping capacity of the heart and improve irregular heartbeat activity. Examples of cardiac glycosides include digitalis, digoxin, and digitoxin.

### Antithrombotic Agents

Antithrombotics inhibit the clotting ability of the blood. There are three main types of antithrombotics—platelet inhibitors, anticoagulants, and thrombolytic agents. In some embodiments, the at least one additional therapeutic agent can be an antithrombotic agent selected from platelet inhibitors, anticoagulants, and thrombolytic agents.

Platelet inhibitors inhibit the clotting activity of platelets, thereby reducing clotting in the arteries. Examples of platelet inhibitors include acetylsalicylic acid (aspirin), ticlopidine, clopidogrel (Plavix®), prasugrel (Effient®), dipyridamole, cilostazol, persantine sulfinpyrazone, dipyridamole, indomethacin, and glycoprotein 11b/111a inhibitors, such as abciximab, tirofiban, and eptifibatide (Integrelin®). Beta blockers and calcium channel blockers also have a platelet-inhibiting effect.

Anticoagulants prevent blood clots from growing larger and prevent the formation of new clots. Examples of anticoagulants include bivalirudin (Angiomax®), warfarin (Coumadin®), unfractionated heparin, low molecular weight heparin, danaparoid, lepirudin, and argatroban.

Thrombolytic agents act to break down an existing blood clot. Examples of thrombolytic agents include streptokinase, urokinase, and tenecteplase (TNK), and tissue plasminogen activator (t-PA).

### Antiarrhythmic Agents

Antiarrhythmic agents are used to treat disorders of the heart rate and rhythm.

Examples of antiarrhythmic agents include amiodarone, dronedarone, quinidine, procainamide, lidocaine, and propafenone. Cardiac glycosides and beta blockers are also used as antiarrhythmic agents.

Combinations with amiodarone and dronedarone are of particular interest (see U.S.

Patent Application Publication No. 2010/0056536 and U.S. Patent Application Publication No. 2011/0183990, the entirety of which are incorporated herein).

### Antihypertensive Agents

Antihypertensive agents are used to treat hypertension, a condition in which the blood pressure is consistently higher than normal. Hypertension is associated with many aspects of cardiovascular disease, including congestive heart failure, atherosclerosis, and clot formation. Examples of antihypertensive agents include alpha-1-adrenergic antagonists, such as prazosin (Minipress®), doxazosin mesylate (Cardura®), prazosin hydrochloride (Minipress®), prazosin, polythiazide (Minizide®), and terazosin hydrochloride (Hytrin®); beta-adrenergic antagonists, such as propranolol (Inderal®), nadolol (Corgard®), timolol (Blocadren®), metoprolol (Lopressor®), and pindolol (Visken®); central alpha-adrenoceptor agonists, such as clonidine hydrochloride (Catapres®), clonidine hydrochloride and chlorthalidone (Clorpres®, Combipres®), guanabenz Acetate (Wytensin®), guanfacine hydrochloride (Tenex®), methyldopa (Aldomet®), methyldopa and chlorothiazide (Aldoclor®), methyldopa and hydrochlorothiazide (Aldoril®); combined alpha/beta-adrenergic antagonists, such as labetalol (Normodyne®, Trandate®), carvedilol (Coreg®); adrenergic neuron blocking agents, such as guanethidine (Ismelin®), reserpine (Serpasil®); central nervous system-acting antihypertensives, such as clonidine (Catapres®), methyldopa (Aldomet®), guanabenz (Wytensin®); anti-angiotensin II agents; ACE inhibitors, such as perindopril (Aceon®) captopril (Capoten®), enalapril (Vasotec®), lisinopril (Prinivil®, Zestril®); angiotensin-II receptor antagonists, such as candesartan (Atacand®), eprosartan (Teveten®), irbesartan (Avapro®), losartan (Cozaar®), telmisartan (Micardis®), valsartan (Diovan®); calcium channel blockers, such as verapamil (Calan®, Isoptin®), diltiazem (Cardizem®), nifedipine (Adalat®, Procardia®); diuretics; direct vasodilators, such as nitroprusside (Nipride®), diazoxide (Hyperstat® IV), hydralazine (Apresoline®), minoxidil (Loniten®), verapamil; and potassium channel activators, such as aprikalim, bimakalim, cromakalim, emakalim, nicorandil, and pinacidil.

### Lipid Lowering Agents

Lipid lowering agents are used to lower the amounts of cholesterol or fatty sugars present in the blood. Examples of lipid lowering agents include bezafibrate (Bezalip®), ciprofibrate (Modalim®), and statins, such as atorvastatin (Lipitor®), fluvastatin (Lescol®), lovastatin (Mevacor®, Altocor®), mevastatin, pitavastatin (Livalo®, Pitava®) pravastatin (Lipostat®), rosuvastatin (Crestor®), and simvastatin (Zocor®).

In this invention, the patient presenting with an acute coronary disease event often suffers from secondary medical conditions such as one or more of a metabolic disorder, a pulmonary disorder, a peripheral vascular disorder, or a gastrointestinal disorder. Such patients can benefit from treatment of a combination therapy comprising administering to the patient a compound as disclosed herein (e.g., Formula I) in combination with at least one therapeutic agent.

### Pulmonary Disorders Combination Therapy

Pulmonary disorder refers to any disease or condition related to the lungs. Examples of pulmonary disorders include, without limitation, asthma, chronic obstructive pulmonary disease (COPD), bronchitis, and emphysema.

Examples of therapeutics agents used to treat pulmonary disorders include bronchodilators including beta2 agonists and anticholinergics, corticosteroids, and electrolyte supplements. Specific examples of therapeutic agents used to treat pulmonary disorders include epinephrine, terbutaline (Brethaire®, Bricanyl®), albuterol (Proventil®), salmeterol (Serevent®, Serevent Diskus®), theophylline, ipratropium bromide (Atrovent®), tiotropium (Spiriva®), methylprednisolone (Solu-Medrol®, Medrol®), magnesium, and potassium.

### Metabolic Disorders Combination Therapy

Examples of metabolic disorders include, without limitation, diabetes, including type I and type II diabetes, metabolic syndrome, dyslipidemia, obesity, glucose intolerance, hypertension, elevated serum cholesterol, and elevated triglycerides.

Examples of therapeutic agents used to treat metabolic disorders include antihypertensive agents and lipid lowering agents, as described in the section “Cardiovascular Agent Combination Therapy” above. Additional therapeutic agents used to treat metabolic disorders include insulin, sulfonylureas, biguanides, alpha-glucosidase inhibitors, and incretin mimetics.

### Peripheral Vascular Disorders Combination Therapy

Peripheral vascular disorders are disorders related to the blood vessels (arteries and veins) located outside the heart and brain, including, for example peripheral arterial disease (PAD), a condition that develops when the arteries that supply blood to the internal organs, arms, and legs become completely or partially blocked as a result of atherosclerosis.

### Gastrointestinal Disorders Combination Therapy

Gastrointestinal disorders refer to diseases and conditions associated with the gastrointestinal tract. Examples of gastrointestinal disorders include gastroesophageal reflux disease (GERD), inflammatory bowel disease (IBD), gastroenteritis, gastritis and peptic ulcer disease, and pancreatitis.

Examples of therapeutic agents used to treat gastrointestinal disorders include proton pump inhibitors, such as pantoprazole (Protonix®), lansoprazole (Prevacid®), esomeprazole (Nexium®), omeprazole (Prilosec®), rabeprazole; H2 blockers, such as cimetidine (Tagamet®), ranitidine (Zantac®), famotidine (Pepcid®), nizatidine (Axid®); prostaglandins, such as misoprostol (Cytotec®); sucralfate; and antacids.

Antibiotics, analgesics, antidepressants and anti-anxiety agents Combination Therapy

Patients presenting with an acute coronary disease event may exhibit conditions that benefit from administration of therapeutic agent or agents that are antibiotics, analgesics, antidepressant and anti-anxiety agents in combination with a compound as disclosed herein (e.g., Formula I).

### Antibiotics

Antibiotics are therapeutic agents that kill, or stop the growth of, microorganisms, including both bacteria and fungi. Example of antibiotic agents include β-Lactam antibiotics, including penicillins (amoxicillin), cephalosporins, such as cefazolin, cefuroxime, cefadroxil (Duricef®), cephalexin (Keflex®), cephradine (Velosef®), cefaclor (Ceclor®), cefuroxime axtel (Ceftin®), cefprozil (Cefzil®), loracarbef (Lorabid®), cefixime (Suprax®), cefpodoxime proxetil (Vantin®), ceftibuten (Cedax®), cefdinir (Omnicef®), ceftriaxone (Rocephin®), carbapenems, and monobactams; tetracyclines, such as tetracycline; macrolide antibiotics, such as erythromycin; aminoglycosides, such as gentamicin, tobramycin, amikacin; quinolones such as ciprofloxacin; cyclic peptides, such as vancomycin, streptogramins, polymyxins; lincosamides, such as clindamycin; oxazolidinoes, such as linezolid; and sulfa antibiotics, such as sulfisoxazole.

### Analgesics

Analgesics are therapeutic agents that are used to relieve pain. Examples of analgesics include opiates and morphinomimetics, such as fentanyl and morphine; paracetamol; NSAIDs, and COX-2 inhibitors. Given the ability of the late sodium channel blockers of the disclosure to treat neuropathic pain via inhibition of the Nay 1.7 and 1.8 sodium channels, combination with analgesics are particularly invisioned. See U.S. Patent Application Publication 20090203707.

### Antidepressant and Anti-Anxiety Agents

Antidepressant and anti-anxiety agents include those agents used to treat anxiety disorders, depression, and those used as sedatives and tranquillers. Examples of antidepressant and anti-anxiety agents include benzodiazepines, such as diazepam, lorazepam, and midazolam; enzodiazepines; barbiturates; glutethimide; chloral hydrate; meprobamate; sertraline (Zoloft®, Lustral®, Apo-Sertral®, Asentra®, Gladem®, Serlift®, Stimuloton®); escitalopram (Lexapro®, Cipralex®); fluoxetine (Prozac®, Sarafem®, Fluctin®, Fontex®, Prodep®, Fludep®, Lovan®); venlafaxine (Effexor® XR, Efexor®); citalopram (Celexa®, Cipramil®, Talohexane®); paroxetine (Paxil®, Seroxat®, Aropax®); trazodone (Desyrel®); amitriptyline (Elavil®); and bupropion (Wellbutrin®, Zyban®).

Accordingly, one aspect of the disclosure provides for a composition comprising the late sodium channel blockers of the disclosure and at least one therapeutic agent. In an alternative embodiment, the composition comprises the late sodium channel blockers of the disclosure and at least two therapeutic agents. In further alternative embodiments, the composition comprises the late sodium channel blockers of the disclosure and at least three therapeutic agents, the late sodium channel blockers of the disclosure and at least four therapeutic agents, or the late sodium channel blockers of the disclosure and at least five therapeutic agents.

The methods of combination therapy include co-administration of a single formulation containing the late sodium channel blockers of the disclosure and therapeutic agent or agents, essentially contemporaneous administration of more than one formulation comprising the late sodium channel blocker of the disclosure and therapeutic agent or agents, and consecutive administration of a late sodium channel blocker of the disclosure and therapeutic agent or agents, in any order, wherein preferably there is a time period where the late sodium channel blocker of the disclosure and therapeutic agent or agents simultaneously exert their therapeutic effect.

E. SYK Inhibitors

The at least one additional therapeutic agent can also be a spleen tyrosine kinase (Syk) inhibitor. In some embodiments, the present invention provides a pharmaceutical composition including a compound of Formula I, at least one additional therapeutic agent that is a spleen tyrosine kinase (Syk) inhibitor, and a pharmaceutically acceptable carrier or excipient.

The SYK inhibitor can be any suitable SYK inhibitor. For example, the SYK inhibitor can be a compound described in U.S. Publication No. 2015/0175616, incorporated by reference herein in its entirety. In some embodiments, the SYK inhibitor can be a compound of Formula B:

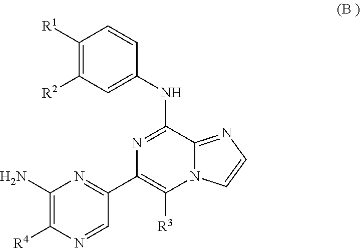

wherein the variables R¹, R², R³ and R⁴ are as defined in U.S. Publication No. 2015/0175616.

In some embodiments, the SYK inhibitor can be:
2-(5-((6-(6-amino-5-methylpyrazin-2-yl)imidazo[1,2-a]pyrazin-8-yl)amino)-2-(4-(oxetan-3-yl)piperazin-1-yl)phenoxy)ethanol,
6-(6-aminopyrazin-2-yl)-N-(4-(4-(oxetan-3-yl)piperazin-1-yl)phenyl)imidazo[1,2-a]pyrazin-8-amine,
2-((4-(4-((6-(6-aminopyrazin-2-yl)imidazo[1,2-a]pyrazin-8-yl)amino)phenyl)piperazin-1-yl)methyl)propane-1,3-diol,
2-(5-((6-(6-aminopyrazin-2-yl)imidazo[1,2-a]pyrazin-8-yl)amino)-2-(4-(oxetan-3-yl)piperazin-1-yl)phenoxy)ethanol,
(R)-(4-(4-((6-(6-aminopyrazin-2-yl)imidazo[1,2-a]pyrazin-8-yl)amino)phenyl)morpholin-2-yl)methanol,
6-(6-aminopyrazin-2-yl)-5-methyl-N-(4-(4-(oxetan-3-yl)piperazin-1-yl)phenyl)imidazo[1,2-a]pyrazin-8-amine, or
6-(6-amino-5-methylpyrazin-2-yl)-N-(4-(4-(oxetan-3-yl)piperazin-1-yl)phenyl)imidazo[1,2-a]pyrazin-8-amine,
or a pharmaceutically acceptable salt, pharmaceutically acceptable co-crystal,
pharmaceutically acceptable ester, stereoisomer, mixture of stereoisomers or tautomer thereof.

One of skill in the art understands that additional therapeutic agents identified above as a member of one class of therapeutic agent useful for treating one disease state, but not listed as a member of the same class of therapeutic agent useful for treating a second disease state, can still be used to treat the second disease state. For example, LOXL inhibitors identified as useful for treating oncology diseases but which are not specifically listed as useful for treating fibrosis, are understood by one of skill in the art as useful for treating fibrosis.

### V. Administration

While it is possible for an active ingredient (i.e., the compound of Formula I and/or the at least one additional therapeutic agent) to be administered alone, it may be preferable to present them as pharmaceutical formulations or pharmaceutical compositions as described below. The formulations, both for veterinary and for human use, of the disclosure comprise at least one of the active ingredients (i.e., the compound of Formula I and/or the at least one additional therapeutic agent), together with one or more acceptable carriers therefor and optionally other therapeutic ingredients. The carrier(s) must be “acceptable” in the sense of being compatible with the other ingredients of the formulation and physiologically innocuous to the recipient thereof.

The compound of Formula I and the at least one additional therapeutic agent may be administered under fed conditions. The term “fed conditions” or variations thereof refers to the consumption or uptake of food, in either solid or liquid forms, or calories, in any suitable form, before or at the same time when the active ingredients are administered. For example, the compound of Formula I and the at least one additional therapeutic agent may be administered to the subject (e.g., a human) within minutes or hours of consuming calories (e.g., a meal). In some embodiments, the compound of Formula I and the at least one additional therapeutic agent may be administered to the subject (e.g., a human) within 5-10 minutes, about 30 minutes, or about 60 minutes of consuming calories.

Each of the compound of Formula I and the at least one additional therapeutic agent can be formulated with conventional carriers and excipients, which will be selected in accord with ordinary practice. Tablets can contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986). Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10.

The therapeutically effective amount of the compound of Formula I and the at least one additional therapeutic agent can be readily determined by a skilled clinician using conventional dose escalation studies. Typically, the compound of Formula I and the at least one additional therapeutic agent will be administered in a dose from 0.01 milligrams to 2 grams. In one embodiment, the dosage will be from about 10 milligrams to 450 milligrams. In another embodiment, the dosage will be from about 25 to about 250 milligrams. In another embodiment, the dosage will be about 50 or 100 milligrams. In one embodiment, the dosage will be about 100 milligrams. It is contemplated that the compound of Formula I and the at least one additional therapeutic agent may be administered once, twice or three times a day. Also, the compound of Formula I and the at least one additional therapeutic agent may be administered once or twice a week, once every two weeks, once every three weeks, once every four weeks, once every five weeks, or once every six weeks.

The therapeutically effective amount of the compound of Formula I and the at least one additional therapeutic agent can be readily determined by a skilled clinician using conventional dose escalation studies. In some embodiments, the compound of Formula I, the composition or the formulation thereof, will be administered in a dose from about 0.01 milligrams (mg) to 2 grams (g), about 0.1 mg to 450 mg, about 0.5 mg to about 250 mg, about 0.5 mg to 100 mg, about 0.5 mg to 50 mg, about 0.5 mg to 40 mg, about 0.5 mg to 30 mg, about 0.5 mg to 20 mg, about 0.5 mg to 10 mg, about 0.5 mg to 5 mg, about 0.5 mg to 4 mg, about 0.5 mg to 3 mg, about 0.5 mg to 2 mg, about 0.5 mg to 1 mg, about 1 mg to 250 mg, about 1 mg to 100 mg, about 1 mg to 50 mg, about 1 mg to 40 mg, about 1 to 35 mg, about 1 mg to 30 mg, about 1 to 25 mg, about 1 mg to 20 mg, about 1 to 15 mg, about 1 mg to 10 mg, about 1 mg to 5 mg, about 1 mg to 4 mg, about 1 mg to 3 mg, or about 1 mg to 2 mg. In another embodiment, the dosage ranges from about 1 mg or 100 mg. In some other embodiment, the dosage ranges from about 1 mg to 30 mg. In certain other embodiment, the dosage ranges from about 1 mg to 20 mg. In one embodiment, the dosage is about 0.5, 1, 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100 mg. It is contemplated that the compound of Formula I and/or the at least one additional therapeutic agent, the composition or the formulation thereof, may be administered once, twice, or three times a day. Also, the compound of Formula I and/or the at least one additional therapeutic agent, the composition or the formulation thereof, may be administered once or twice a week, once every two weeks, once every three weeks, once every four weeks, once every five weeks, or once every six weeks.

In certain other embodiments, the compound of Formula I and/or the at least one additional therapeutic agent, the composition or the formulation thereof, is administered at between about 25 mg to about 800 mg per subject. In some embodiments, the dosage is about 50 mg, about 100 mg, at about 150 m, about 200 mg, about 250 mg, about 300 mg, about 350 mg, about 400 mg, about 450 mg, about 500 mg, about 550 mg, about 600 mg, about 650 mg, about 700 mg, about 750 mg, or about 800 mg per subject, including any range in between these values. In some embodiments, the compound of Formula I and/or the at least one additional therapeutic agent, the composition or the formulation thereof, of the above dosage is administered once a week, once every two weeks, once every three weeks, once a month, once every two months, once every three months, or once every six months. In one embodiment, the compound of Formula I and/or the at least one additional therapeutic agent, the composition or the formulation thereof is delivered by intravenous administration (which may be referred to as intravenous infusion) or subcutaneous administration (which may be referred to as subcutaneous injection). In some embodiments, the compound of Formula I and/or the at least one additional therapeutic agent, the composition or the formulation thereof, is administered subcutaneously at about 75 mg or 125 mg once a week. In certain embodiment, the compound of Formula I and/or the at least one additional therapeutic agent, the composition or the formulation thereof, is administered intravenously at about 200 mg or 700 mg once a month. In additional embodiment, the compound of Formula I and/or the at least one additional therapeutic agent, the composition or the formulation thereof is administered subcutaneously (i.e. subcutaneous injection) at about 75 mg once a week. In one embodiment, the compound of Formula I and/or the at least one additional therapeutic agent, the composition or the formulation thereof is administered subcutaneously at about 125 mg once a week.

The pharmaceutical composition for the active ingredient can include those suitable for the foregoing administration routes. The formulations can conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations suitable for oral administration can be presented as discrete units such as capsules, sachets or tablets each containing a predetermined amount of the compound of Formula I and/or the at least one additional therapeutic agent; as a powder or granules; as a solution or a suspension in an aqueous or non-aqueous liquid; or as an oil-in-water liquid emulsion or a water-in-oil liquid emulsion. The compound of Formula I and/or the at least one additional therapeutic agent may also be administered as a bolus, electuary or paste. In certain embodiments, the compound of Formula I and/or the at least one additional therapeutic agent may be administered as a subcutaneous injection.

A tablet can be made by compression or molding, optionally with one or more accessory ingredients. Compressed tablets can be prepared by compressing in a suitable machine the active ingredient in a free-flowing form such as a powder or granules, optionally mixed with a binder, lubricant, inert diluent, preservative, or surface active agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

The compound of Formula I and/or the at least one additional therapeutic agent can be administered by any route appropriate to the condition. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. In certain embodiments, the active ingredients are orally bioavailable and can therefore be dosed orally. In certain cases, the compound of Formula I and/or the at least one additional therapeutic agent, is administered with food. In one embodiment, the patient is human.

When used in combination in the methods disclosed herein, the compound of Formula I and the at least one additional therapeutic agent can be administered together in a single pharmaceutical composition, or separately (either concurrently or sequentially) in more than one pharmaceutical composition. In certain embodiments, the compound of Formula I and the at least one additional therapeutic agent are administered together. In other embodiments, the compound of Formula I and the at least one additional therapeutic agent are administered separately. In some aspects, the compound of Formula I is administered prior to the at least one additional therapeutic agent. In some aspects, the at least one additional therapeutic agent is administered prior to the compound of Formula I. When administered separately, the compound of Formula I and the at least one additional therapeutic agent can be administered to the patient by the same or different routes of delivery. For example, the compound of Formula I may be administered orally and the at least one additional therapeutic agent may be administered subcutaneously. In some embodiments, the compound of Formula I and the at least one additional therapeutic agent are administered in different tablets, but at substantially the same time.

In some embodiments, the compound of Formula I and/or the at least one additional therapeutic agent can be administered by any useful route and means, such as by oral or parenteral (e.g., intravenous) administration. Therapeutically effective amounts of the compound of Formula I and/or the at least one additional therapeutic agent are from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day, or such as from about 0.3 μg to about 30 mg per day, or such as from about 30 μg to about 300 μg per day.

Therapeutically effective amounts of the compound of Formula I and the at least one additional therapeutic agent are also from about 0.01 mg per dose to about 1000 mg per dose, such as from about 0.01 mg per dose to about 100 mg per dose, or such as from about 0.1 mg per dose to about 100 mg per dose, or such as from about 1 mg per dose to about 100 mg per dose, or such as from about 1 mg per dose to about 10 mg per dose. Other therapeutically effective amounts of the compound of Formula I and the at least one additional therapeutic agent are about 1 mg per dose, or about 2, 3, 4, 5, 6, 7, 8, 9, 10, 15, 20, 25, 30, 35, 40, 45, 50, 55, 60, 65, 70, 75, 80, 85, 90, 95, or about 100 mg per dose. Other therapeutically effective amounts of the compound of Formula I and the at least one additional therapeutic agent are about 100 mg per dose, or about 125, 150, 175, 200, 225, 250, 275, 300, 350, 400, 450, or about 500 mg per dose. A single dose can be administered hourly, daily, or weekly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, or once every 4 weeks. A single dose can also be administered once every month.

The frequency of dosage of the compound of Formula I and the at least one additional therapeutic agent will be determined by the needs of the individual patient and can be, for example, once per day or twice, or more times, per day.

Administration can be intermittent, with a period of several or more days during which a patient receives a daily dose of the compound of Formula I and the at least one additional therapeutic agent, followed by a period of several or more days during which a patient does not receive a daily dose of the compound of Formula I and the at least one additional therapeutic agent. For example, a patient can receive a dose of the compound of Formula I and the at least one additional therapeutic agent every other day, or three times per week. Again by way of example, a patient can receive a dose of the compound of Formula I and the at least one additional therapeutic agent each day for a period of from 1 to 14 days, followed by a period of 7 to 21 days during which the patient does not receive a dose of the compound of Formula I and the at least one additional therapeutic agent, followed by a subsequent period (e.g., from 1 to 14 days) during which the patient again receives a daily dose of the compound of Formula I and the at least one additional therapeutic agent. Alternating periods of administration of the compound of Formula I, followed by non-administration of the compound of Formula I and the at least one additional therapeutic agent, can be repeated as clinically required to treat the patient.

As described more fully herein, the compound of Formula I can be administered with one or more additional therapeutic agent(s) to a human being suffering from a particular disease condition. The additional therapeutic agent(s) can be administered to the human being at the same time as the compound of Formula I, or before or after administration of the compound of Formula I. In some embodiments, the present invention provides the compound of Formula I, for use in a method of treating or preventing a disease condition, wherein the compound of Formula I is administered simultaneously, separately or sequentially with one or more additional therapeutic agents for treating the disease condition. In some embodiments, the present invention provides use of the compound of Formula I for the manufacture of a medicament for the treatment of a disease condition, wherein the compound of Formula I is administered simultaneously, separately or sequentially with one or more additional therapeutic agents for treating the disease condition.

### VI. Examples

### Example 1: Model System

Bleomycin-induced pulmonary fibrosis in mice is a recognized, standard model system for IPF and other pulmonary fibrotic disorders. See, for example, Harrison and Lazo (1987) J. Pharmacol. Exp. Ther. 243:1185-1194; Walters and Kleeberger (2008) Current Protocols Pharmacol. 40:5.46.1-5.46.17. This system is used to study the effects of a combination of agents as described herein, on the course and outcome of lung fibrosis.

In brief, lung fibrosis is induced in male C57B/L6 mice by oropharyngeal administration of bleomycin. For bleomycin administration, animals are anaesthetized and suspended on their backs at an approximately 600 angle with a rubber band running under the upper incisors. The tongue is held with one arm of a set of padded forceps, thereby opening the airway. Bleomycin solution is introduced into the back of the oral cavity by pipette, and the tongue and mouth are held open until the liquid was no longer visible in the mouth.

Mice may also administered a combination of agents either before (Prevention study) or after (Treatment study) bleomycin treatment.

### Example 2: Prevention Study

In this study, mice are administered a compound of Formula (I), an additional therapeutic agent as described herein, or a combination of a compound of Formula (I) and an additional therapeutic agent and then administered bleomycin and allowed to develop pulmonary fibrosis. Suitable control agents may also be administered. The studies may be performed as described in U.S. Publication No. 2011/0044981, which is incorporated by reference in its entirety herein, with design modifications to account for combination agents.

### Example 3: Treatment Study for Pulmonary Fibrosis

In this study, mice are administered bleomycin and allowed to develop pulmonary fibrosis, then treated with a compound of Formula (I), an additional therapeutic agent as described herein, or a combination of a compound of Formula (I) and an additional therapeutic acgent. Suitable control agents may also be administered. The studies may be performed as described in U.S. Publication No. 2011/0044981 with design modifications to account for combination agents.

### Example 4: Treatment Study for Rheumatoid Arthritis

Additionally, the compounds and combinations described herein may be assessed in other models of inflammatory diseases, such as rheumatoid arthritis. For example, the collagen induced arthritis model may be used to assess the compounds and combinations described herein. Exemplary methods may be found in Di Paolo et al., Nature Chem. Bio., vol. 7, pp. 41-50 (2010) and Liu et al., JPET, vol. 338, pp. 154-163 (2011).

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

Where a conflict exists between the instant application and a reference provided herein, the instant application shall dominate.

What is claimed is:

1. A pharmaceutical composition comprising
a therapeutically effective amount of an autotaxin inhibitor compound of Formula I: 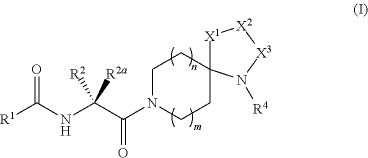
or a pharmaceutically acceptable salt thereof;
at least one additional therapeutic agent; and
a pharmaceutically acceptable carrier or excipient,
wherein
X¹ and X² are each independently selected from one or more of C₁₋₂ alkyl, C═O, NR³, or O;
X³ is independently selected from one or more of C₁₋₂ alkyl, C═O, NR³, O, or CR¹⁰R¹¹;
m and n are each independently selected from 0, 1 or 2;
R¹ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G¹ substituents;
R² is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G² substituents;
R^{2a} is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G^{2a} substituents;
R² and R^{2a} are each independently a linear structure, or, R² and R^{2a} are taken together with the carbon atom to which they are attached to form a 3-12 membered saturated or unsaturated ring, wherein said ring optionally includes one or more additional heteroatoms selected from O, N, or S(O)ₘ₁;
R³ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G³ substituents;
R⁴ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, heteroaryl-C₃₋₁₂heterocycloalkyl-, or pyridine-N-oxide, any of which is optionally substituted with one or more independent G⁴ substituents;
G¹, G², G^{2a}, G³, and G⁴ are each independently selected from one or more of H, D, halo, —CN, —CD₃, —OCD₃, -oxo-, —CF₃, —OCF₃, —OCHF₂, —NR⁵R⁶, —NO₂, —B(OH)₂, —PO(OR¹²)₂, —PO(OR¹²)R¹³, —CONR¹²OH, —C₀₋₁₂alkyl, —C₂₋₁₂alkenyl, —C₂₋₁₂alkynyl, C₃₋₁₂cycloalkyl-C₀₋₁₂alkyl-, C₃₋₁₂heterocycloalkyl-C₀₋₁₂alkyl-, aryl-C₀₋₁₂alkyl-, heteroaryl-C₀₋₁₂alkyl-, —OC₀₋₁₂alkyl, —S(O)ₙ₁R¹², —C(O)R¹², —C(O)NR¹²R¹³, —C(O)—C(O)NR¹²R¹³, —C(O)OR¹², —C(O)—C(O)OR¹², —OC(O)R¹², —NR¹²C(O)R¹³, —NR¹²C(O)OR¹³, —NR¹²S(O)₂R¹³, —(CR¹⁴R¹⁵)C(O)R¹³, —(CR¹⁴R¹⁵)C(O)OR¹², —(CR¹⁴R¹⁵)C(O)NR¹²R¹³, —(CR¹⁴R¹⁵)ₙ₁S(O)₂NR¹²R¹³, —(CR¹⁴R¹⁵)ₙ₁NR¹²R¹³, —(CR¹⁴R¹⁵)ₙ₁OR¹², —(CR¹⁴R¹⁵)ₙ₁S(O)ₙ₂R¹², —NR¹⁶C(O)NR¹²R¹³, —NR¹⁶S(O)₂NR¹²R¹³ or —NR¹⁶S(O)NR¹²R¹³, any of which is optionally substituted with one or more independent Q¹ substituents;
Q¹ is selected from H, D, halo, —CN, —CD₃, —OCD₃, -oxo-, —CF₃, —OCF₃, —OCHF₂, —NO₂, —B(OH)₂, —PO(OR¹⁷)₂, —PO(OR¹⁷)R¹⁸, NR¹⁷R¹⁸, —CONR¹⁷OH, C₀₋₁₂alkyl-, —C₂₋₁₂ alkenyl, —C₂₋₁₂alkynyl, aryl-C₀₋₁₂alkyl-, heteroaryl-C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂cycloalkyl-, heteroaryl-C₃₋₁₂cycloalkyl-, C₃₋₁₂heterocycloalkyl-C₃₋₁₂cycloalkyl-, C₃₋₁₂cycloalkyl-C₃₋₁₂cycloalkyl-, C₁₋₁₂alkyl-C₃₋₁₂heterocycloalkyl-, C₃₋₁₂heterocycloalkyl-C₃₋₁₂heterocycloalkyl-, aryl-C₃₋₁₂heterocycloalkyl-, heteroaryl-C₃₋₁₂heterocycloalkyl-, —OC₀₋₁₂alkyl, —C(O)—C(O)NR¹⁷R¹⁸, —C(O)—C(O)OR¹⁷, —OC(O)R¹⁷, —NR¹⁷C(O)R¹⁸, —NR¹⁷S(O)₂R¹⁸, —(CR¹⁹R²⁰)ₙ₃C(O)R¹⁷, —(CR¹⁹R²⁰)ₙ₃C(O)OR¹⁷, —(CR¹⁹R²⁰)ₙ₃C(O)NR¹⁷R¹⁸, —(CR¹⁹R²⁰)ₙ₃S(O)₂NR¹⁷R¹⁸, —(CR¹⁹R²⁰)ₙ₃NR¹⁷R¹⁸, —(CR¹⁹R²⁰)ₙ₃OR¹⁷, —(CR¹⁹R²⁰)ₙ₃S(O)ₙ₄R¹⁷, —NR²¹C(O)NR¹⁷R¹⁸, —NR²¹S(O)₂NR¹⁷R¹⁸ or —NR²¹S(O)NR¹⁷R¹⁸, any of which is optionally substituted with one or more independent Q² substituents;
Q² is selected from one or more of H, D, halo, —CN, -oxo-, —CD₃, —OCD₃, —CF₃, —OCF₃, —OCHF₂, —NO₂, —B(OH)₂, —PO(OR²⁷)₂, —PO(OR²⁷)R²⁸—CONR²⁷OH, —CONR²⁷R²⁸C₀₋₁₂alkyl-, —C₂₋₁₂ alkenyl, —C₂₋₁₂ alkynyl, —OC₀₋₁₂alkyl, aryl-C₀₋₁₂alkyl-, heteroaryl-C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂cycloalkyl-, heteroaryl-C₃₋₁₂cycloalkyl-, C₃₋₁₂heterocycloalkyl-C₃₋₁₂cycloalkyl-, C₃₋₁₂cycloalkyl-C₃₋₁₂cycloalkyl-, C₁₋₁₂alkyl-C₃₋₁₂heterocycloalkyl-, C₃₋₁₂heterocycloalkyl-C₃₋₁₂heterocycloalkyl-, aryl-C₃₋₁₂heterocycloalkyl-, heteroaryl-C₃₋₁₂heterocycloalkyl-, C(O)—C(O)NR²⁷R²⁸, —C₀₋₁₂alkylC(O)OR²⁷, —C(O)—C(O)OR²⁷, —OC(O)R²⁷, —NR²⁷C(O)R²⁸, —NR²⁷C(O)OR²⁸—NR²⁷S(O)₂R²⁸, (CR²⁹R³⁰)ₙ₅C(O)R²⁷, —(CR²⁹R³⁰)ₙ₅C(O)OR²⁷, —(CR²⁹R³⁰)ₙ₅C(O)NR²⁷R²⁸, (CR²⁹R³⁰)ₙ₅S(O)₂NR²⁷R²⁸, —(CR²⁹R³⁰)ₙ₅NR²⁷R²⁸, —(CR²⁹R³⁰)ₙ₅OR²⁷, —(CR²⁹R³⁰)ₙ₅S(O)ₙ₆R²⁷, —NR³⁰C(O)NR²⁷R²⁸, —NR³⁰S(O)₂NR²⁷R²⁸ or —NR³⁰S(O)NR²⁷R²⁸ substituents, any of which may be optionally substituted;
R⁵, R⁶, R¹⁰, R¹¹, R¹², R¹³, R¹⁴, R¹⁵, and R¹⁶ are each independently selected from one or more of H, C₁₋₆alkyl-, C₃₋₈cycloalkyl-C₀₋₆alkyl-, C₃₋₈heterocycloalkyl-C₀₋₆alkyl-, aryl-C₀₋₆alkyl-, aryl-C₃₋₈cycloalkyl-, aryl-C₃₋₈heterocycloalkyl-, heteroaryl-C₁₋₆alkyl-, heteroaryl-C₃₋₈cycloalkyl- or heteroaryl-C₃₋₈heterocycloalkyl-, any of which may be optionally substituted;
R¹⁷, R¹⁸, R¹⁹, R²⁰, R²¹, R²⁷, R²⁸, R²⁹, and R³⁰ are each independently selected from H, C₁₋₆alkyl-, C₃₋₈cycloalkyl-C₀₋₆alkyl-, C₃₋₈heterocycloalkyl-C₀₋₆alkyl-, aryl-C₀₋₆alkyl-, aryl-C₃₋₈cycloalkyl-, aryl-C₃₋₈heterocycloalkyl-, heteroaryl-C₁₋₆alkyl-, heteroaryl-C₃₋₈cycloalkyl- or heteroaryl-C₃₋₈heterocycloalkyl-, any of which may be optionally substituted;
—NR⁵R⁶ and —NR¹²R¹³ are each independently a linear structure, or, R⁵ and R⁶, or R¹² and R¹³, respectively, are taken together with the nitrogen atom to which they are attached to form a 3-12 membered saturated or unsaturated ring, wherein said ring optionally includes one or more additional heteroatoms selected from O, N, or S(O)ₘ₂;
—CR¹⁰R¹¹ and —CR¹⁴R¹⁵ are each independently a linear structure, or, R¹⁰ and R¹¹, or R¹⁴ and R¹⁵ respectively, are taken together with the carbon atom to which they are attached to form a 3-12 membered saturated or unsaturated ring, wherein said ring optionally includes one or more heteroatoms selected from O, N, or S(O)ₘ₃;
—CR¹⁹R²⁰ is a linear structure, or, R¹⁹ and R²⁰ are taken together with the carbon atom to which they are attached to form a 3-12 membered saturated or unsaturated ring, wherein said ring optionally includes one or more additional heteroatoms selected from O, N, or S(O)ₘ₄;
—NR¹⁷R¹⁸ is a linear structure, or, R¹⁷ and R¹⁸ are taken together with the nitrogen atom to which they are attached to form a 3-12 membered saturated or unsaturated ring, wherein said ring optionally includes one or more additional heteroatoms selected from O, N, or S(O)ₘ₅;
—CR²⁹R³⁰ is a linear structure, or, R²⁹ and R³⁰ are taken together with the carbon atom to which they are attached to form a 3-12 membered saturated or unsaturated ring, wherein said ring optionally includes one or more additional heteroatoms selected from O, N, or S(O)ₘ₆; and
—NR²⁷R²⁸ is a linear structure, or, R²⁷ and R²⁸ are taken together with the nitrogen atom to which they are attached to form a 3-12 membered saturated or unsaturated ring, wherein said ring optionally includes one or more additional heteroatoms selected from O, N, or S(O)ₘ₇;
wherein m1, m2, m3, m4, m5, m6, m7, n1, n2, n3, n4, n5 and n6 are each independently selected from 0, 1 or 2.

2. The composition of claim 1, wherein
X¹ is selected from C₁₋₂ alkyl and C═O;
X² is selected from NR³ and O;
X³ is independently selected from one or more of C₁₋₂ alkyl or C═O;
m and n are each independently selected from 0, 1 or 2;
R¹ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G¹ substituents selected from H, halo, —CN, —CF₃, —OCF₃, —OCHF₂, —NR⁵R⁶, —NO₂, —C₀₋₁₂alkyl, —C₂₋₁₂alkenyl, —C₂₋₁₂alkynyl, C₃₋₁₂cycloalkyl-C₀₋₁₂alkyl-, C₃₋₁₂heterocycloalkyl-C₀₋₁₂alkyl-, aryl-C₀₋₁₂alkyl-, heteroaryl-C₀₋₁₂alkyl-, —OC₀₋₁₂alkyl, —S(O)ₙ₁R¹², —C(O)R¹², —C(O)NR¹²R¹³, —C(O)OR¹², —OC(O)R¹², —NR¹²C(O)R¹³, —NR¹²C(O)OR¹³, —NR¹²S(O)₂R¹³, or —(CR¹⁴R¹⁵)ₙ₁S(O)₂NR¹²R¹³;
R² is selected from C₀₋₁₂alkyl-, C₃₋₁₂cycloalkyl-C₀₋₁₂alkyl-,C₃₋₁₂heterocycloalkyl-C₀₋₁₂alkyl-, aryl-C₀₋₁₂alkyl-, or heteroaryl-C₀₋₁₂alkyl-, any of which is optionally substituted with one or more independent G² substituents selected from H, halo, —CN, —CF₃, —OCF₃, —OCHF₂, —NR⁵R⁶, —NO₂, —C₀₋₁₂alkyl, —C₂₋₁₂alkenyl, —C₂₋₁₂alkynyl, C₃₋₁₂cycloalkyl-C₀₋₁₂alkyl-, C₃₋₁₂heterocycloalkyl-C₀₋₁₂alkyl-, aryl-C₀₋₁₂alkyl-, heteroaryl-C₀₋₁₂alkyl-, —OC₀₋₁₂alkyl, —S(O)ₙ₁R¹², —C(O)R¹², —C(O)NR¹²R¹³, —C(O)OR¹², —OC(O)R¹², —NR¹²C(O)R¹³, —NR¹²C(O)OR¹³, NR¹²S(O)₂R¹³, or —(CR¹⁴R¹⁵)ₙ₁S(O)₂NR¹²R¹³;
R^{2a} is selected from C₀₋₁₂alkyl-, or C₃₋₁₂heterocycloalkyl-C₀₋₁₂alkyl-;
R² and R^{2a} are each independently a linear structure, or, R² and R^{2a} are taken together with the carbon atom to which they are attached to form a 3-12 membered saturated or unsaturated ring, wherein said ring optionally includes one or more additional heteroatoms selected from O, N, or S(O)ₘ₁;
R³ is selected from C₀₋₁₂alkyl-, or C₃₋₁₂cycloalkyl-C₀₋₁₂alkyl-, optionally substituted with one or more independent G³ substituents selected from H, halo, —CN, —CF₃, —OCF₃, —OCHF₂, —NR⁵R⁶, C₃₋₁₂cycloalkyl-C₀₋₁₂alkyl-, —S(O)ₙ₁R¹², —C(O)R¹², —C(O)NR¹²R¹³, —C(O)OR¹², —OC(O)R¹², —NR¹²C(O)R¹³, —NR¹²C(O)OR¹³, NR¹²S(O)₂R¹³, or —(CR¹⁴R¹⁵)ₙ₁S(O)₂NR¹²R¹³;
R⁴ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, heteroaryl-C₃₋₁₂heterocycloalkyl-, or pyridine-N-oxide, any of which is optionally substituted with one or more independent G⁴ substituents selected from H, D, halo, —CN, —CD₃, —OCD₃, -oxo-, —CF₃, —OCF₃, —OCHF₂, —NR⁵R⁶, —NO₂, —B(OH)₂, —PO(OR¹²)₂, —PO(OR¹²)R¹³, —CONR¹²OH, —C₀₋₁₂alkyl, —C₂₋₁₂alkenyl, —C₂₋₁₂alkynyl, C₃₋₁₂cycloalkyl-C₀₋₁₂alkyl-, C₃₋₁₂heterocycloalkyl-C₀₋₁₂alkyl-, aryl-C₀₋₁₂alkyl-, heteroaryl-C₀₋₁₂alkyl-, —OC₀₋₁₂alkyl, —S(O)ₙ₁R¹², C(O)R¹², —C(O)NR¹²R¹³, —C(O)OR¹², —OC(O)R¹², —NR¹²C(O)R¹³, —NR¹²C(O)OR¹³, —NR¹²S(O)₂R¹³, or —(CR¹⁴R¹⁵)ₙ₁S(O)₂NR¹²R¹³, wherein —OC₀₋₁₂alkyl is optionally substituted with one or more independent Q¹ substituents selected from halo, —CN, —CF₃, —OCF₃, —OCHF₂, NR¹⁷R¹⁸, —C₀₋₁₂alkyl, aryl-C₀₋₁₂alkyl-, heteroaryl-C₀₋₁₂alkyl-, C₃₋₁₂cycloalkyl-C₀₋₁₂alkyl-, C₃₋₁₂heterocycloalkyl-C₀₋₁₂alkyl-, —OC₀₋₁₂alkyl, —OC(O)R¹⁷, —NR¹⁷C(O)R¹⁸, —NR¹⁷S(O)₂R¹⁸, —(CR¹⁹R²⁰)ₙ₃C(O)R¹⁷, —(CR¹⁹R²⁰)ₙ₃C(O)OR¹⁷, —(CR¹⁹R²⁰)ₙ₃C(O)NR¹⁷R¹⁸, —(CR¹⁹R²⁰)ₙ₃S(O)₂NR¹⁷R¹⁸, —(CR¹⁹R²⁰)ₙ₃NR¹⁷R¹⁸, or —(CR¹⁹R²⁰)ₙ₃OR¹⁷, —R⁵, R⁶, R¹², R¹³, R¹⁴ and R¹⁵ are each independently selected from one or more of H, C₁₋₆alkyl-, C₃₋₈cycloalkyl-C₀₋₆alkyl-,C₃₋₈heterocycloalkyl-C₀₋₆alkyl-, aryl-C₀₋₆alkyl-, or heteroaryl-C₁₋₆alkyl-;
R¹⁷, R¹⁸, R¹⁹, and R²⁰ are each independently selected from H, C₁₋₆alkyl-, C₃₋₈cycloalkyl-C₀₋₆alkyl-, C₃₋₈heterocycloalkyl-C₀₋₆alkyl-, aryl-C₀₋₆alkyl-, or heteroaryl-C₁₋₆alkyl-;
—NR⁵R⁶ and —NR¹²R¹³ are each independently a linear structure;
—CR¹⁴R¹⁵ is a linear structure;
—CR¹⁹R²⁰ is a linear structure; and
—NR¹⁷R¹⁸ is a linear structure;
wherein m1, n1 and n3 are each independently selected from 0, 1 or 2.

3. The composition of claim 1, wherein
X¹ is selected from C₁₋₂ alkyl and C═O;
X² is selected from NR³ and O;
X³ is independently selected from one or more of C₁₋₂ alkyl or C═O;
m and n are each 1;
R¹ is selected from C₀₋₁₂alkyl-, C₃₋₁₂cycloalkyl-C₀₋₁₂alkyl-, aryl-C₀₋₁₂alkyl-, or heteroaryl-C₀₋₁₂alkyl-, any of which is optionally substituted with one or more independent G¹ substituents selected from H, halo, —CN, —CF₃, —OCF₃, —OCHF₂, —C₀₋₁₂alkyl, C₃₋₁₂cycloalkyl-C₀₋₁₂alkyl-, or —OC₀₋₁₂alkyl;
R² is selected from C₀₋₁₂alkyl-, C₃₋₁₂cycloalkyl-C₀₋₁₂alkyl-, or C₃₋₁₂heterocycloalkyl-C₀₋₁₂alkyl-, any of which is optionally substituted with one or more independent G² substituents selected from H or —OC₀₋₁₂alkyl;
R^{2a} is selected from C₀₋₁₂alkyl-, or C₃₋₁₂heterocycloalkyl-C₀₋₁₂alkyl-;
R² and R^{2a} are each independently a linear structure, or, R² and R^{2a} are taken together with the carbon atom to which they are attached to form a 3-12 membered saturated or unsaturated ring, wherein said ring optionally includes one or more additional heteroatoms selected from O, N, or S(O)ₘ₁;
R³ is selected from C₀₋₁₂alkyl-, or C₃₋₁₂cycloalkyl-C₀₋₁₂alkyl-, optionally substituted with one or more independent G³ substituents selected from H, —CN, —NR⁵R⁶, C₃₋₁₂cycloalkyl-C₀₋₁₂alkyl-, —S(O)ₙ₁R¹², or —C(O)OR¹²;
R⁴ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, heteroaryl-C₀₋₁₂alkyl-, or pyridine-N-oxide, any of which is optionally substituted with one or more independent G⁴ substituents selected from H, D, halo, —CN, —OCD₃, -oxo-, —CF₃, —OCF₃, —NR⁵R⁶, —B(OH)₂, —C₀₋₁₂alkyl, aryl-C₀₋₁₂alkyl-, heteroaryl-C₀₋₁₂alkyl-, —OC₀₋₁₂alkyl, —C(O)R¹², —S(O)ₙ₁R¹², —C(O)NR¹²R¹³, —C(O)OR¹², —NR¹²C(O)R¹³, —NR¹²C(O)OR¹³, or —NR¹²S(O)₂R¹³, wherein —OC₀₋₁₂alkyl is optionally substituted with one or more independent Q¹ substituents selected from —CN, NR¹⁷R¹⁸, C₀₋₁₂alkyl-, C₃₋₁₂heterocycloalkyl-C₀₋₁₂alkyl-, —OC₀₋₁₂alkyl, —(CR¹⁹R²⁰)ₙ₃C(O)OR¹⁷, or —(CR¹⁹R²⁰)ₙ₃C(O)NR¹⁷R¹⁸;
R⁵, R⁶, R¹², and R¹³ are each independently selected from one or more of H, C₁₋₆alkyl-, C₃₋₈cycloalkyl-C₀₋₆alkyl-, or C₃₋₈heterocycloalkyl-C₀₋₆alkyl-;
R¹⁷, R¹⁸, R¹⁹, and R²⁰ are each independently selected from H, or C₁₋₆alkyl-;
—NR⁵R⁶ and —NR¹²R¹³ are each independently a linear structure;
—CR¹⁴R¹⁵ is a linear structure;
—CR¹⁹R²⁰ is a linear structure; and
—NR¹⁷R¹⁸ is a linear structure;
wherein m1, n1 and n3 are each independently selected from 0, 1 or 2.

4. The composition of claim 1, wherein the compound is of Formula Ia: 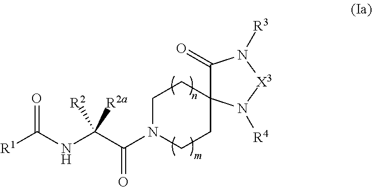
or a pharmaceutically acceptable salt thereof.

5. The composition of claim 1, wherein the compound is of Formula Id: 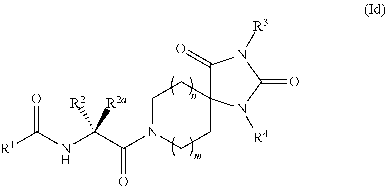
or a pharmaceutically acceptable salt thereof.

6. The composition of claim 1, wherein the compound is of Formula Ih: 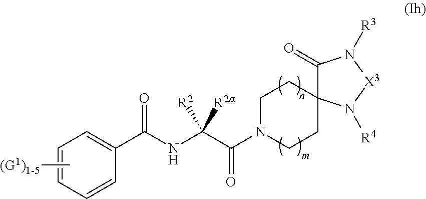
or a pharmaceutically acceptable salt thereof.

7. The composition of claim 1, wherein the compound is of Formula Ik: 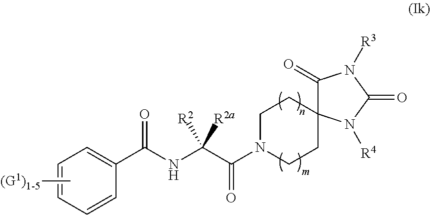
or a pharmaceutically acceptable salt thereof.

8. The composition of claim 1, wherein the compound is of Formula Io: 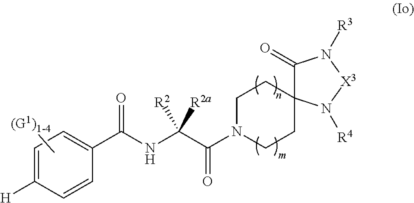
or a pharmaceutically acceptable salt thereof.

9. The composition of claim 1, wherein the compound is of the Formula Ip: 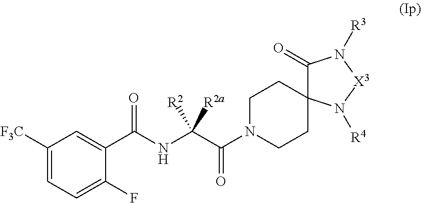
or a pharmaceutically acceptable salt thereof.

10. The composition of claim 1, wherein the compound is of the Formula Iq: 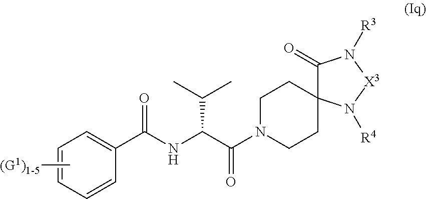
or a pharmaceutically acceptable salt thereof.

11. The composition of claim 1, wherein the compound is of the Formula Ir: 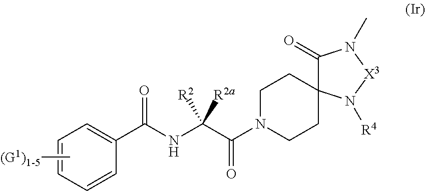
or a pharmaceutically acceptable salt thereof.

12. The composition of claim 1, wherein the compound is of the Formula Is: 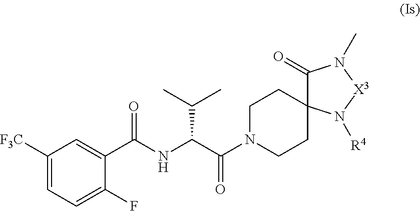
or a pharmaceutically acceptable salt thereof.

13. The composition of claim 1, wherein the compound is of the Formula It: 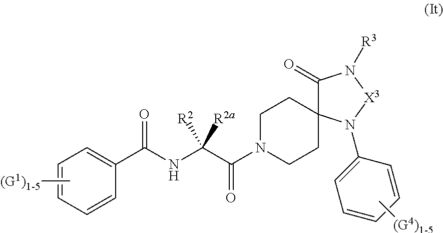
or a pharmaceutically acceptable salt thereof.

14. The composition of claim 1, wherein the compound is of the Formula Iu: 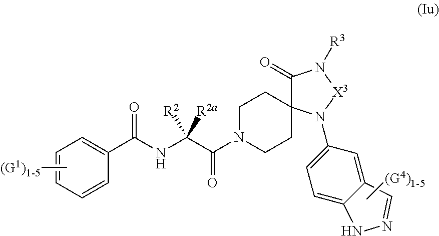
or a pharmaceutically acceptable salt thereof.

15. The composition of claim 1, wherein the compound is of the Formula Iv: 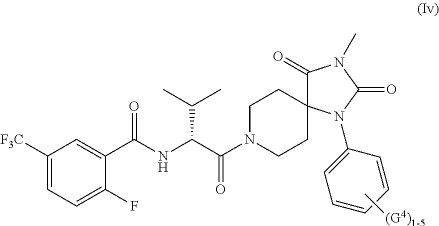
or a pharmaceutically acceptable salt thereof.

16. The composition of claim 1, wherein the compound is of the Formula Iw: 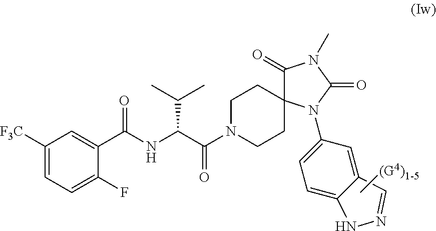
or a pharmaceutically acceptable salt thereof.

17. The composition of claim 1, wherein
R¹ is selected from t-Bu, cyclohexane, adamantyl, phenyl, pyridine or thiazole, each optionally substituted with one or more independent G¹ substituents selected from H, F, Cl, —CN, —CF₃, —OCF₃, —OCH₂F, methyl, ethyl, cyclopropyl, or methoxy;
R² is selected from H, methyl, ethyl, isopropyl, sec-butyl, cyclopropyl, cyclobutyl, cyclopentyl, cyclohexyl, or tetrahydro-2H-pyran, wherein the ethyl group is optionally substitued with one or more G¹ substituent selected from —OMe;
R^{2a} is selected from H or tetrahydro-2H-pyran;
or R² and R^{2a} are taken together with the carbon atom to which they are attached to form a cyclopropane or oxetane;
R³ is methyl, ethyl, propyl, or cyclopropylmethyl, optionally substituted with one or more independent G³ substituents selected from —CN, —NMe₂, cyclopropyl, —SO₂Me, or —COOH; and
each G⁴ substituent is selected from H, D, F, Cl, —CN, —OCD₃, oxo, —CF₃, —OCF₃, —NH(azetidine), —NH(oxetane), —B(OH)₂, Me, triazole, tetrazole, —OMe, -OEt, —SO₂Me, —C(O)NH₂, —COOH, —C(O)OMe, —NHC(O)-cyclopropane, —NHC(O)OMe, or —NHSO₂Me, wherein the —OMe and -OEt groups are optionally substituted with one or more independent Q¹ substituents selected from —CN, NMe₂, Me, azetidine, oxetane, —OH, —COOH, or —C(O)NH₂.

18. The composition of claim 1, wherein the autotaxin inhibitor of Formula I has the structure selected from the group consisting of:
(R)—N-(1-Cyclohexyl-2-(3-methyl-2,4-dioxo-1-phenyl-1,3,8-triazaspiro[4.5]decan-8-yl)-2-oxoethyl)-3-methylbenzamide;
(R)-3-Fluoro-N-(3-methyl-1-(3-methyl-2,4-dioxo-1-phenyl-1,3,8-triazaspiro[4.5]decan-8-yl)-1-oxobutan-2-yl)benzamide;
(R)-2-Fluoro-3-methyl-N-(3-methyl-1-(3-methyl-2,4-dioxo-1-phenyl-1,3,8-triazaspiro[4.5]decan-8-yl)-1-oxobutan-2-yl)benzamide;
2-Fluoro-3-methyl-N-((2R,3R)-3-methyl-1-(3-methyl-2,4-dioxo-1-phenyl-1,3,8-triazaspiro[4.5]decan-8-yl)-1-oxopentan-2-yl)benzamide;
(R)—N-(1-(3-(Cyanomethyl)-1-(4-methoxyphenyl)-2,4-dioxo-1,3,8-triazaspiro[4.5]decan-8-yl)-3-methyl-1-oxobutan-2-yl)-5-ethyl-2-fluorobenzamide;
(R)-2-Fluoro-N-(1-(1-(3-fluoro-4-methoxyphenyl)-3-methyl-2,4-dioxo-1,3,8-triazaspiro[4.5]decan-8-yl)-3-methyl-1-oxobutan-2-yl)-5-(trifluoromethyl)benzamide;
(R)—N-(1-(1-(4-Cyanophenyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1-(3-Cyanophenyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(3-methyl-1-(3-methyl-1-(4-(methylsulfonyl)phenyl)-2,4-dioxo-1,3,8-triazaspiro[4.5]decan-8-yl)-1-oxobutan-2-yl)-5-(trifluoromethyl)benzamide;
(R)-2-Fluoro-N-(1-(1-(3-methoxyphenyl)-3-methyl-2,4-dioxo-1,3,8-triazaspiro[4.5]decan-8-yl)-3-methyl-1-oxobutan-2-yl)-5-(trifluoromethyl)benzamide;
(R)-2-Fluoro-N-(3-methyl-1-(3-methyl-1-(3-methyl-2-oxo-2,3-dihydrobenzo[d]oxazol-5-yl)-2,4-dioxo-1,3,8-triazaspiro[4.5]decan-8-yl)-1-oxobutan-2-yl)-5-(trifluoromethyl)benzamide;
(R)-2-Fluoro-N-(3-methyl-1-(3-methyl-1-(1-methyl-1H-indazol-5-yl)-2,4-dioxo-1,3,8-triazaspiro[4.5]decan-8-yl)-1-oxobutan-2-yl)-5-(trifluoromethyl)benzamide;
(R)-2-Fluoro-N-(3-methyl-1-(3-methyl-1-(2-methyl-2H-indazol-5-yl)-2,4-dioxo-1,3,8-triazaspiro[4.5]decan-8-yl)-1-oxobutan-2-yl)-5-(trifluoromethyl)benzamide;
(R)-2-Fluoro-N-(1-(1-(imidazo[1,2-a]pyridin-6-yl)-3-methyl-2,4-dioxo-1,3,8-triazaspiro[4.5]decan-8-yl)-3-methyl-1-oxobutan-2-yl)-5-(trifluoromethyl)benzamide trifluoroacetic acid salt;
(R)-2-Fluoro-N-(3-methyl-1-(3-methyl-1-(3-(methylsulfonyl)phenyl)-2,4-dioxo-1,3,8-triazaspiro[4.5]decan-8-yl)-1-oxobutan-2-yl)-5-(trifluoromethyl)benzamide;
(R)-2-Fluoro-N-(3-methyl-1-(3-methyl-1-(1-methyl-1H-pyrazol-3-yl)-2,4-dioxo-1,3,8-triazaspiro[4.5]decan-8-yl)-1-oxobutan-2-yl)-5-(trifluoromethyl)benzamide;
(R)-2-Fluoro-N-(3-methyl-1-(3-methyl-1-(1-methyl-1H-pyrazol-4-yl)-2,4-dioxo-1,3,8-triazaspiro[4.5]decan-8-yl)-1-oxobutan-2-yl)-5-(trifluoromethyl)benzamide;
(R)-5-Ethyl-2-fluoro-N-(3-methyl-1-(3-methyl-1-(3-methylimidazo[1,5-a]pyridin-6-yl)-2,4-dioxo-1,3,8-triazaspiro[4.5]decan-8-yl)-1-oxobutan-2-yl)benzamide;
(R)-2-Fluoro-N-(3-methyl-1-(3-methyl-1-(2-methyl-1H-benzo[d]imidazol-6-yl)-2,4-dioxo-1,3,8-triazaspiro[4.5]decan-8-yl)-1-oxobutan-2-yl)-5-(trifluoromethyl)benzamide trifluoroacetic acid salt;
(R)-2-Fluoro-N-(3-methyl-1-(3-methyl-2,4-dioxo-1-(1H-pyrrolo[2,3-b]pyridin-5-yl)-1,3,8-triazaspiro[4.5]decan-8-yl)-1-oxobutan-2-yl)-5-(trifluoromethyl)benzamide;
(R)—N-(1-(1-(3,4-Dihydro-2H-benzo[b][1,4]oxazin-7-yl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(3-methyl-1-(3-methyl-2,4-dioxo-1-(6-oxo-1,6-dihydropyridin-3-yl)-1,3,8-triazaspiro[4.5]decan-8-yl)-1-oxobutan-2-yl)-5-(trifluoromethyl)benzamide;
(R)-2-Fluoro-N-(3-methyl-1-(3-methyl-1-(1-methyl-6-oxo-1,6-dihydropyridin-3-yl)-2,4-dioxo-1,3,8-triazaspiro[4.5]decan-8-yl)-1-oxobutan-2-yl)-5-(trifluoromethyl)benzamide;
(R)—N-(1-Cyclopentyl-2-(3-methyl-1-(1-methyl-6-oxo-1,6-dihydropyridin-3-yl)-2,4-dioxo-1,3,8-triazaspiro[4.5]decan-8-yl)-2-oxoethyl)-2-fluoro-5-(trifluoromethyl)benzamide;
(R)-2-Fluoro-N-(1-(1-(6-methoxypyridin-3-yl)-3-methyl-2,4-dioxo-1,3,8-triazaspiro[4.5]decan-8-yl)-3-methyl-1-oxobutan-2-yl)-5-(trifluoromethyl)benzamide;
(R)-2-Fluoro-N-(3-methyl-1-(3-methyl-2,4-dioxo-1-(4-(trifluoromethoxy)phenyl)-1,3,8-triazaspiro[4.5]decan-8-yl)-1-oxobutan-2-yl)-5-(trifluoromethyl)benzamide;
(R)-2-Fluoro-N-(3-methyl-1-(3-methyl-2,4-dioxo-1-(5-benzofuran)-1,3,8-triazaspiro[4.5]decan-8-yl)-1-oxobutan-2-yl)-5-(trifluoromethyl)benzamide;
(R)-2-Fluoro-N-(3-methyl-1-(3-methyl-2,4-dioxo-1-(2-oxo-2,3-dihydrobenzo[d]oxozol-6-yl)-1,3,8-triazaspiro[4.5]decan-8-yl)-1-oxobutan-2-yl)-5-(trifluoromethyl)benzamide;
(R)-2-Fluoro-N-(3-methyl-1-(3-methyl-2,4-dioxo-1-(2-oxo-2,3-dihydrobenzo[d]oxozol-5-yl)-1,3,8-triazaspiro[4.5]decan-8-yl)-1-oxobutan-2-yl)-5-(trifluoromethyl)benzamide;
(R)-2-Fluoro-N-(3-methyl-1-(3-methyl-2,4-dioxo-1-(3-oxo-3,4-dihydro-2H-benzo[b][1,4]oxazin-7-yl)-1,3,8-triazaspiro[4.5]decan-8-yl)-1-oxobutan-2-yl)-5-(trifluoromethyl)benzamide;
(R)-5-Ethyl-2-fluoro-N-(3-methyl-1-(3-methyl-2,4-dioxo-1-(2-oxo-2,3-dihydrobenzo[d]oxozol-6-yl)-1,3,8-triazaspiro[4.5]decan-8-yl)-1-oxobutan-2-yl)benzamide;
(R)—N-(1-(1-(1H-Benzo[d][1,2,3]triazol-5-yl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(3-Methyl-1-(3-methyl-2,4-dioxo-1-phenyl-1,3,8-triazaspiro[4.5]decan-8-yl)-1-oxobutan-2-yl)-3-(trifluoromethyl)benzamide;
(R)-3-Ethyl-2-fluoro-N-(3-methyl-1-(3-methyl-2,4-dioxo-1-phenyl-1,3,8-triazaspiro[4.5]decan-8-yl)-1-oxobutan-2-yl)benzamide;
(R)-3,4-Dichloro-N-(3-methyl-1-(3-methyl-2,4-dioxo-1-phenyl-1,3,8-triazaspiro[4.5]decan-8-yl)-1-oxobutan-2-yl)benzamide;
(R)-2,5-Dichloro-N-(3-methyl-1-(3-methyl-2,4-dioxo-1-phenyl-1,3,8-triazaspiro[4.5]decan-8-yl)-1-oxobutan-2-yl)benzamide;
5-Ethyl-2-fluoro-N-((2R,3S)-3-methoxy-1-(3-methyl-2,4-dioxo-1-phenyl-1,3,8-triazaspiro[4.5]decan-8-yl)-1-oxobutan-2-yl)benzamide;
3-Ethyl-5-fluoro-N-((2R,3S)-3-methoxy-1-(3-methyl-2,4-dioxo-1-phenyl-1,3,8-triazaspiro[4.5]decan-8-yl)-1-oxobutan-2-yl)benzamide;
(R)-5-Ethyl-2-fluoro-N-(3-methyl-1-(3-methyl-2,4-dioxo-1-phenyl-1,3,8-triazaspiro[4.5]decan-8-yl)-1-oxobutan-2-yl)benzamide;
5-Ethyl-2-fluoro-N-((2R,3R)-3-methoxy-1-(1-(4-methyoxyphenyl)-3-methyl-2,4-dioxo-1,3,8-triazaspiro[4.5]decan-8-yl)-1-oxobutan-2-yl)benzamide;
3-fluoro-N-((2R,3R)-1-(1-(4-methoxyphenyl)-3-methyl-2,4-dioxo-1,3,8-triazaspiro[4.5]decan-8-yl)-3-methyl-1-oxobutan-2-yl)-5-methylbenzamide;
(R)-5-Cyclopropyl-2-fluoro-N-(3-methyl-1-(3-methyl-2,4-dioxo-1-phenyl-1,3,8-triazaspiro[4.5]decan-8-yl)-1-oxobutan-2-yl)benzamide;
(R)-5-Chloro-2-fluoro-N-(3-methyl-1-(3-methyl-2,4-dioxo-1-phenyl-1,3,8-triazaspiro[4.5]decan-8-yl)-1-oxobutan-2-yl)benzamide;
(R)-2-Fluoro-N-(3-methyl-1-(3-methyl-2,4-dioxo-1-phenyl-1,3,8-triazaspiro[4.5]decan-8-yl)-1-oxobutan-2-yl)-5-(trifluoromethyl)benzamide;
(R)-5-Cyclopropyl-2-fluoro-N-(1-(1-(4-methoxyphenyl)-3-methyl-2,4-dioxo-1,3,8-triazaspiro[4.5]decan-8-yl)-3-methyl-1-oxobutan-2-yl)benzamide;
(R)-3-Ethyl-N-(3-methyl-1-(3-methyl-2,4-dioxo-1-phenyl-1,3,8-triazaspiro[4.5]decan-8-yl)-1-oxobutan-2-yl)benzamide;
(R)-3-Ethyl-5-fluoro-N-(3-methyl-1-(3-methyl-2,4-dioxo-1-phenyl-1,3,8-triazaspiro[4.5]decan-8-yl)-1-oxobutan-2-yl)benzamide;
(R)-3,5-Dichloro-N-(3-methyl-1-(3-methyl-2,4-dioxo-1-phenyl-1,3,8-triazaspiro[4.5]decan-8-yl)-1-oxobutan-2-yl)benzamide;
5-Cyclopropyl-2-fluoro-N-((2R,3R)-3-methoxy-1-(1-(4-methyoxyphenyl)-3-methyl-2,4-dioxo-1,3,8-triazaspiro[4.5]decan-8-yl)-1-oxobutan-2-yl)benzamide;
(R)-5-Ethyl-2-fluoro-N-(1-(1-(4-methoxyphenyl)-3-methyl-2,4-dioxo-1,3,8-triazaspiro[4.5]decan-8-yl)-3-methyl-1-oxobutan-2-yl)benzamide;
(R)-2-Fluoro-N-(1-(1-(4-methoxyphenyl)-3-methyl-2,4-dioxo-1,3,8-triazaspiro[4.5]decan-8-yl)-3-methyl-1-oxobutan-2-yl)-5-(trifluoromethyl)benzamide;
(R)—N-(1-(1-(1H-indazol-5-yl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1-(1H-Indazol-6-yl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-5-cyclopropyl-2-fluorobenzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-5-ethyl-2-fluorobenzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-2-fluoro-5-methylbenzamide;
(R)—N-(2-(1-(1H-Indazol-5-yl)-3-methyl-2,4-dioxo-1,3,8-triazaspiro[4.5]decan-8-yl)-1-cyclopentyl-2-oxoethyl)-2-fluoro-5-(trifluoromethyl)benzamide;
(R)—N-(2-(1-(1H-Indazol-5-yl)-3-methyl-2,4-dioxo-1,3,8-triazaspiro[4.5]decan-8-yl)-1-cyclobutyl-2-oxoethyl)-2-fluoro-5-(trifluoromethyl)benzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-3-(trifluoromethyl)benzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-3-chloro-5-(trifluoromethyl)benzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-2-methyl-5-(trifluoromethyl)benzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-2-fluoro-5-(trifluoromethoxy)benzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-2-fluoro-5-methoxybenzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-5-(difluoromethoxy)-2-fluorobenzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-2,5-dichlorobenzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-2,5-difluorobenzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-1-admantanecarboxly amide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-2-chloro-5-(trifluoromethyl)benzamide;
(S)—N-(1-(1-(1H-Indazol-5-yl)-3-methyl-2,4-dioxo-1,3,8-triazaspiro[4.5]decan-8-yl)-3-methyl-1-oxobutan-2-yl)-2-fluoro-5-(trifluoromethyl)benzamide;
N-(2-(1-(1H-Indazol-5-yl)-3-methyl-2,4-dioxo-1,3,8-triazaspiro[4.5]decan-8-yl)-2-oxoethyl)-2-fluoro-5-(trifluoromethyl)benzamide;
N-(1-(1-(1H-Indazol-5-yl)-3-methyl-2,4-dioxo-1,3,8-triazaspiro[4.5]decane-8-carbonyl)cyclopropyl)-2-fluoro-5-(trifluoromethyl)benzamide;
(R)—N-(1-(1-(1H-Indazol-5-yl)-3-methyl-2,4-dioxo-1,3,8-triazaspiro[4.5]decan-8-yl)-1-oxopropan-2-yl)-2-fluoro-5-(trifluoromethyl)benzamide;
N-(3-(1-(1H-Indazol-5-yl)-3-methyl-2,4-dioxo-1,3,8-triazaspiro[4.5]decane-8-carbonyl)oxetan-3-yl)-2-fluoro-5-(trifluoromethyl)benzamide;
N-(1-(1-(1H-Indazol-5-yl)-3-methyl-2,4-dioxo-1,3,8-triazaspiro[4.5]decan-8-yl)-2-methyl-1-oxopropan-2-yl)-2-fluoro-5-(trifluoromethyl)benzamide;
(R)—N-(1-(1-(Cyclopropylmethyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1-Cyclopropyl-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(3-methyl-1-(3-methyl-2,4-dioxo-1-(tetrahydro-2H-pyran-4-yl)-1,3,8-triazaspiro[4.5]decan-8-yl)-1-oxobutan-2-yl)-5-(trifluoromethyl)benzamide;
2-Fluoro-N—((R)-3-methyl-1-(3-methyl-2,4-dioxo-1-((S)-1-phenylethyl)-1,3,8-triazaspiro[4.5]decan-8-yl)-1-oxobutan-2-yl)-5-(trifluoromethyl)benzamide;
(R)-2-Fluoro-N-(3-methyl-1-(3-methyl-2,4-dioxo-1-((tetrahydro-2H-pyran-4-yl)methyl)-1,3,8-triazaspiro[4.5]decan-8-yl)-1-oxobutan-2-yl)-5-(trifluoromethyl)benzamide;
2-Fluoro-N—((R)-3-methyl-1-(3-methyl-2,4-dioxo-1-((R)-1-phenylethyl)-1,3,8-triazaspiro[4.5]decan-8-yl)-1-oxobutan-2-yl)-5-(trifluoromethyl)benzamide;
(R)—N-(1-(1-(4-(Azetidin-3-ylmethoxy)phenyl)-3-methyl-2,4-dioxo-1,3,8-triazaspiro[4.5]decan-8-yl)-3-methyl-1-oxobutan-2-yl)-2-fluoro-5-(trifluoromethyl)benzamide trifluoroacetic acid salt;
(R)—N-(1-(1-(4-(cyanomethoxy)phenyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(3-methyl-1-(3-methyl-1-(4-(oxetan-3-ylmethoxy)phenyl)-2,4-dioxo-1,3,8-triazaspiro[4.5]decan-8-yl)-1-oxobutan-2-yl)-5-(trifluoromethyl)benzamide;
(R)-2-Fluoro-N-(1-(1-(4-(2-hydroxyethoxy)phenyl)-3-methyl-2,4-dioxo-1,3,8-triazaspiro[4.5]decan-8-yl)-3-methyl-1-oxobutan-2-yl)-5-(trifluoromethyl)benzamide;
(R)—N-(1-(1-(4-(2-(Dimethylamino)ethoxy)phenyl)-3-methyl-2,4-dioxo-1,3,8-triazaspiro[4.5]decan-8-yl)-3-methyl-1-oxobutan-2-yl)-2-fluoro-5-(trifluoromethyl)benzamide trifluoroacetic acid salt;
(R)—N-(1-(1-(4-(Azetidin-3-ylamino)phenyl)-3-methyl-2,4-dioxo-1,3,8-triazaspiro[4.5]decan-8-yl)-3-methyl-1-oxobutan-2-yl)-2-fluoro-5-(trifluoromethyl)benzamide trifluoroacetic acid salt;
(R)-2-Fluoro-N-(3-methyl-1-(3-methyl-1-(4-(oxetan-3-ylamino)phenyl)-2,4-dioxo-1,3,8-triazaspiro[4.5]decan-8-yl)-1-oxobutan-2-yl)-5-(trifluoromethyl)benzamide;
(R)—N-(1-(1-(4-Cyanobenzyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1-Benzyl-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(1-(1-(4-fluorobenzyl)-3-methyl-2,4-dioxo-1,3,8-triazaspiro[4.5]decan-8-yl)-3-methyl-1-oxobutan-2-yl)-5-(trifluoromethyl)benzamide;
(R)-2-Fluoro-N-(1-(1-(4-methoxybenzyl)-3-methyl-2,4-dioxo-1,3,8-triazaspiro[4.5]decan-8-yl)-3-methyl-1-oxobutan-2-yl)-5-(trifluoromethyl)benzamide;
2-Fluoro-N-((2R)-3-methyl-1-(3-methyl-2,4-dioxo-1-(1-phenylethyl)-1,3,8-triazaspiro[4.5]decan-8-yl)-1-oxobutan-2-yl)-5-(trifluoromethyl)benzamide;
(R)-2-Fluoro-N-(3-methyl-1-(3-methyl-1-(4-(methylsulfonyl)benzyl)-2,4-dioxo-1,3,8-triazaspiro[4.5]decan-8-yl)-1-oxobutan-2-yl)-5-(trifluoromethyl)benzamide;
(R)-2-Fluoro-N-(1-(1-((2-methoxypyridin-4-yl)methyl)-3-methyl-2,4-dioxo-1,3,8-triazaspiro[4.5]decan-8-yl)-3-methyl-1-oxobutan-2-yl)-5-(trifluoromethyl)benzamide;
(R)-2-Fluoro-N-(3-methyl-1-(3-methyl-1-((2-methylpyridin-4-yl)methyl)-2,4-dioxo-1,3,8-triazaspiro[4.5]decan-8-yl)-1-oxobutan-2-yl)-5-(trifluoromethyl)benzamide;
(R)—N-(1-(1-((2,3-Dihydrobenzo[b][1,4]dioxin-6-yl)methyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(1-(1-((6-methoxypyridin-3-yl)methyl)-3-methyl-2,4-dioxo-1,3,8-triazaspiro[4.5]decan-8-yl)-3-methyl-1-oxobutan-2-yl)-5-(trifluoromethyl)benzamide;
(R)—N-(1-(1-((1H-Indazol-5-yl)methyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1-(4-Chlorobenzyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(3-methyl-1-(3-methyl-2,4-dioxo-1-(quinoxalin-6-ylmethyl)-1,3,8-triazaspiro[4.5]decan-8-yl)-1-oxobutan-2-yl)-5-(trifluoromethyl)benzamide;
(R)—N-(1-(1-(3-Cyanobenzyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(1-(1-(imidazo[1,2-a]pyridin-7-ylmethyl)-3-methyl-2,4-dioxo-1,3,8-triazaspiro[4.5]decan-8-yl)-3-methyl-1-oxobutan-2-yl)-5-(trifluoromethyl)benzamide;
(R)-2-Fluoro-N-(3-methyl-1-(3-methyl-1-(3-(methylsulfonyl)benzyl)-2,4-dioxo-1,3,8-triazaspiro[4.5]decan-8-yl)-1-oxobutan-2-yl)-5-(trifluoromethyl)benzamide;
(R)—N-(1-(1-(3-Chlorobenzyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(3-methyl-1-(3-methyl-1-((3-methyl-2-oxo-2,3-dihydrobenzo[d]oxazol-5-yl)methyl)-2,4-dioxo-1,3,8-triazaspiro[4.5]decan-8-yl)-1-oxobutan-2-yl)-5-(trifluoromethyl)benzamide;
(R)-2-Fluoro-N-(3-methyl-1-(3-methyl-1-((2-methyl-1H-benzo[d]imidazol-6-yl)methyl)-2,4-dioxo-1,3,8-triazaspiro[4.5]decan-8-yl)-1-oxobutan-2-yl)-5-(trifluoromethyl)benzamide;
(R)-2-Fluoro-N-(1-(1-(2-fluoro-5-methoxybenzyl)-3-methyl-2,4-dioxo-1,3,8-triazaspiro[4.5]decan-8-yl)-3-methyl-1-oxobutan-2-yl)-5-(trifluoromethyl)benzamide;
(R)-2-Fluoro-N-(1-(1-(2-fluoro-4-methoxybenzyl)-3-methyl-2,4-dioxo-1,3,8-triazaspiro[4.5]decan-8-yl)-3-methyl-1-oxobutan-2-yl)-5-(trifluoromethyl)benzamide;
(R)—N-(1-Cyclopropyl-2-(3-methyl-2,4-dioxo-1-(quinoxalin-6-ylmethyl)-1,3,8-triazaspiro[4.5]decan-8-yl)-2-oxoethyl)-2-fluoro-5-(trifluoromethyl)benzamide;
(R)—N-(1-Cyclobutyl-2-(3-methyl-2,4-dioxo-1-(quinoxalin-6-ylmethyl)-1,3,8-triazaspiro[4.5]decan-8-yl)-2-oxoethyl)-2-fluoro-5-(trifluoromethyl)benzamide;
(R)-2-Fluoro-N-(3-methyl-1-(3-methyl-2,4-dioxo-1-(pyrimidin-2-ylmethyl)-1,3,8-triazaspiro[4.5]decan-8-yl)-1-oxobutan-2-yl)-5-(trifluoromethyl)benzamide;
(R)—N-(1-Cyclopentyl-2-(3-methyl-2,4-dioxo-1-(quinoxalin-6-ylmethyl)-1,3,8-triazaspiro[4.5]decan-8-yl)-2-oxoethyl)-2-fluoro-5-(trifluoromethyl)benzamide;
(R)—N-(1-Cyclopentyl-2-(3-methyl-1-((2-methyl-[1,2,4]triazolo[1,5-a]pyridin-7-yl)methyl)-2,4-dioxo-1,3,8-triazaspiro[4.5]decan-8-yl)-2-oxoethyl)-2-fluoro-5-(trifluoromethyl)benzamide;
(R)-2-Fluoro-N-(3-methyl-1-(3-methyl-1-((2-methyl-[1,2,4]triazolo[1,5-a]pyridin-7-yl)methyl)-2,4-dioxo-1,3,8-triazaspiro[4.5]decan-8-yl)-1-oxobutan-2-yl)-5-(trifluoromethyl)benzamide;
(R)-2-Fluoro-N-(3-methyl-1-(3-methyl-1-((1-methyl-1H-pyrazol-4-yl)methyl)-2,4-dioxo-1,3,8-triazaspiro[4.5]decan-8-yl)-1-oxobutan-2-yl)-5-(trifluoromethyl)benzamide;
(R)-2-Fluoro-N-(3-methyl-1-(3-methyl-1-((2-methylbenzo[d]oxazol-5-yl)methyl)-2,4-dioxo-1,3,8-triazaspiro[4.5]decan-8-yl)-1-oxobutan-2-yl)-5-(trifluoromethyl)benzamide;
(R)-2-Fluoro-N-(3-methyl-1-(3-methyl-1-((4-methylthiazol-2-yl)methyl)-2,4-dioxo-1,3,8-triazaspiro[4.5]decan-8-yl)-1-oxobutan-2-yl)-5-(trifluoromethyl)benzamide;
(R)-2-Fluoro-N-(3-methyl-1-(3-methyl-1-((2-methylbenzo[d]oxazol-6-yl)methyl)-2,4-dioxo-1,3,8-triazaspiro[4.5]decan-8-yl)-1-oxobutan-2-yl)-5-(trifluoromethyl)benzamide;
(R)—N-(1-(1-(2-Chloro-4-methoxybenzyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(3-methyl-1-(3-methyl-1-((1-methyl-6-oxo-1,6-dihydropyridin-3-yl)methyl)-2,4-dioxo-1,3,8-triazaspiro[4.5]decan-8-yl)-1-oxobutan-2-yl)-5-(trifluoromethyl)benzamide;
(R)—N-(1-(1-((1-Acetylazetidin-3-yl)methyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1,3-Dimethyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1-Ethyl-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(1-(1-isopentyl-3-methyl-2,4-dioxo-1,3,8-triazaspiro[4.5]decan-8-yl)-3-methyl-1-oxobutan-2-yl)-5-(trifluoromethyl)benzamide;
(R)—N-(1-(1-Cyclopentyl-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(3-methyl-1-(3-methyl-2,4-dioxo-1,3,8-triazaspiro[4.5]decan-8-yl)-1-oxobutan-2-yl)-5-(trifluoromethyl)benzamide;
(R)-Methyl-4-(8-(2-(2-fluoro-5-(trifluoromethyl)benzamido)-3-methylbutanoyl)-3-methyl-2,4-dioxo-1,3,8-triazaspiro[4.5]decan-1-yl)phenylcarbamate;
(R)—N-(1-(1-(4-(Cyclopropanecarboxamido)phenyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-3-Methyl-N-(2-(3-methyl-4-oxo-1-phenyl-1,3,8-triazaspiro[4.5]decan-8-yl)-2-oxo-1-(tetrahydro-2H-pyran-4-yl)ethyl)benzamide;
(S)-3-Methyl-N-(2-(3-methyl-4-oxo-1-phenyl-1,3,8-triazaspiro[4.5]decan-8-yl)-2-oxo-1-(tetrahydro-2H-pyran-4-yl)ethyl)benzamide;
(R)—N-(1-Cyclohexyl-2-(3-methyl-4-oxo-1-phenyl-1,3,8-triazaspiro[4.5]decan-8-yl)-2-oxoethyl)-6-methylpicolinamide trifluoroacetic acid salt;
(R)—N,3-Dimethyl-N-(3-methyl-1-(3-methyl-4-oxo-1-phenyl-1,3,8-triazaspiro[4.5]decan-8-yl)-1-oxobutan-2-yl)benzamide;
5-Ethyl-2-fluoro-N-((2R,3S)-3-methoxy-1-(3-methyl-4-oxo-1-phenyl-1,3,8-triazaspiro[4.5]decane-8-yl)-1-oxobutan-2-yl)benzamide;
N-((2R,3S)-3-Methoxy-1-(3-methyl-4-oxo-1-phenyl-1,3,8-triazaspiro[4.5]decan-8-yl)-1-oxobutan-2-yl)-3-methylbenzamide;
3-Ethyl-N-((2R,3S)-3-methoxy-1-(3-methyl-4-oxo-1-phenyl-1,3,8-triazaspiro[4.5]decan-8-yl)-1-oxobutan-2-yl)benzamide;
3-Ethyl-5-fluoro-N-((2R,3S)-3-methoxy-1-(3-methyl-4-oxo-1-phenyl-1,3,8-triazaspiro[4.5]decan-8-yl)-1-oxobutan-2-yl)benzamide;
(R)-2-Methyl-N-(3-methyl-1-(3-methyl-4-oxo-1-phenyl-1,3,8-triazaspiro[4.5]decan-8-yl)-1-oxobutan-2-yl)isonicotinamide;
(R)-3-Ethyl-N-(3-methyl-1-(3-methyl-4-oxo-1-phenyl-1,3,8-triazaspiro[4.5]decan-8-yl)-1-oxobutan-2-yl)benzamide;
(R)-4-Fluoro-3-methyl-N-(3-methyl-1-(3-methyl-4-oxo-1-phenyl-1,3,8-triazaspiro[4.5]decan-8-yl)-1-oxobutan-2-yl)benzamide;
(R)-3-Fluoro-5-methyl-N-(3-methyl-1-(3-methyl-4-oxo-1-phenyl-1,3,8-triazaspiro[4.5]decan-8-yl)-1-oxobutan-2-yl)benzamide;
(R)-2-Fluoro-5-methyl-N-(3-methyl-1-(3-methyl-4-oxo-1-phenyl-1,3,8-triazaspiro[4.5]decan-8-yl)-1-oxobutan-2-yl)benzamide;
(R)-3-Cyclopropyl-N-(3-methyl-1-(3-methyl-4-oxo-1-phenyl-1,3,8-triazaspiro[4.5]decan-8-yl)-1-oxobutan-2-yl)benzamide;
(R)-3-Chloro-4-cyano-N-(3-methyl-1-(3-methyl-4-oxo-1-phenyl-1,3,8-triazaspiro[4.5]decan-8-yl)-1-oxobutan-2-yl)benzamide;
(R)-5-Ethyl-2-fluoro-N-(3-methyl-1-(3-methyl-4-oxo-1-phenyl-1,3,8-triazaspiro[4.5]decan-8-yl)-1-oxobutan-2-yl)benzamide;
(R)—N-(1-(1-(4-Carbamoylphenyl)-3-methyl-4-oxo-1,3,8-triazaspiro[4.5]decan-8-yl)-3-methyl-1-oxobutan-2-yl)-2-fluoro-3-methylbenzamide;
(R)—N-(1-(1-(3-Carbamoylphenyl)-3-methyl-4-oxo-1,3,8-triazaspiro[4.5]decan-8-yl)-3-methyl-1-oxobutan-2-yl)-2-fluoro-3-methylbenzamide;
(R)-4-(8-(2-(2-Fluoro-3-methylbenzamido)-3-methylbutanoyl)-3-methyl-4-oxo-1,3,8-triazaspiro[4.5]decan-1-yl)phenylboronic acid;
(R)-3-(8-(2-(2-Fluoro-3-methylbenzamido)-3-methylbutanoyl)-3-methyl-4-oxo-1,3,8-triazaspiro[4.5]decan-1-yl)phenylboronic acid;
(R)-4-(8-(2-(2-Fluoro-5-(trifluoromethyl)benzamido)-3-methylbutanoyl)-3-methyl-4-oxo-1,3,8-triazaspiro[4.5]decan-1-yl)benzoic acid;
(R)-4-(8-(2-(2-Fluoro-3-methylbenzamido)-3-methylbutanoyl)-3-methyl-4-oxo-1,3,8-triazaspiro[4.5]decan-1-yl)benzoic acid;
(R)-4-(8-(2-(2-Fluoro-5-(trifluoromethyl)benzamido)-3-methylbutanoyl)-3-methyl-4-oxo-1,3,8-triazaspiro[4.5]decan-1-yl)-2-methoxybenzoic acid;
(R)-4-(8-(2-(5-Ethyl-2-fluorobenzamido)-3-methylbutanoyl)-3-methyl-4-oxo-1,3,8-triazaspiro[4.5]decan-1-yl)benzoic acid;
(R)-4-(8-(2-(5-Cyclopropyl-2-fluorobenzamido)-3-methylbutanoyl)-3-methyl-4-oxo-1,3,8-triazaspiro[4.5]decan-1-yl)benzoic acid;
(R)-2-Chloro-4-(8-(2-(2-fluoro-5-(trifluoromethyl)benzamido)-3-methylbutanoyl)-3-methyl-4-oxo-1,3,8-triazaspiro[4.5]decan-1-yl)benzoic acid;
(R)-4-(3-(Cyclopropylmethyl)-8-(2-(2-fluoro-5-(trifluoromethyl)benzamido)-3-methylbutanoyl)-4-oxo-1,3,8-triazaspiro[4.5]decan-1-yl)benzoic acid;
(R)-4-(8-(2-Cyclopentyl-2-(2-fluoro-5-(trifluoromethyl)benzamido)acetyl)-3-methyl-4-oxo-1,3,8-triazaspiro[4.5]decan-1-yl)benzoic acid;
(R)—N-(1-(1-(4-Carbamoylphenyl)-3-methyl-4-oxo-1,3,8-triazaspiro[4.5]decan-8-yl)-3-methyl-1-oxobutan-2-yl)-2-fluoro-5-(trifluoromethyl)benzamide;
(R)-4-(8-(2-(2-Fluoro-5-(trifluoromethyl)benzamido)-3-methylbutanoyl)-3-methyl-4-oxo-1,3,8-triazaspiro[4.5]decan-1-yl)-2-methylbenzoic acid;
(R)-4-(8-(2-(2-Fluoro-5-(trifluoromethyl)benzamido)-3,3-dimethylbutanoyl)-3-methyl-4-oxo-1,3,8-triazaspiro[4.5]decan-1-yl)benzoic acid;
(R)-4-(8-(2-(3-Chloro-5-(trifluoromethyl)benzamido)-3-methylbutanoyl)-3-methyl-4-oxo-1,3,8-triazaspiro[4.5]decan-1-yl)benzoic acid;
(R)-4-(3-Methyl-8-(3-methyl-2-(2-methyl-5-(trifluoromethyl)benzamido)butanoyl)-4-oxo-1,3,8-triazaspiro[4.5]decan-1-yl)benzoic acid;
(R)-4-(8-(2-(2-Fluoro-5-(trifluoromethoxy)benzamido)-3-methylbutanoyl)-3-methyl-4-oxo-1,3,8-triazaspiro[4.5]decan-1-yl)benzoic acid;
(R)-4-(8-(2-(2-Fluoro-5-methoxybenzamido)-3-methylbutanoyl)-3-methyl-4-oxo-1,3,8-triazaspiro[4.5]decan-1-yl)benzoic acid;
(R)-4-(8-(2-(1-Admantanecarboxamido)-3-methylbutanoyl)-3-methyl-4-oxo-1,3,8-triazaspiro[4.5]decan-1-yl)benzoic acid;
(R)-4-(8-(2-(5-(Difluoromethoxy)-2-fluorobenzamido)-3-methylbutanoyl)-3-methyl-4-oxo-1,3,8-triazaspiro[4.5]decan-1-yl)benzoic acid;
(R)-4-(8-(2-(5-(Difluoromethoxy)-2-fluorobenzamido)-3-methylbutanoyl)-3-methyl-2,4-dioxo-1,3,8-triazaspiro[4.5]decan-1-yl)benzoic acid;
(R)-4-(8-(2-(2-Fluoro-5-(trifluoromethoxy)benzamido)-3-methylbutanoyl)-3-methyl-2,4-dioxo-1,3,8-triazaspiro[4.5]decan-1-yl)benzoic acid;
(R)-2-(4-(8-(2-(5-Ethyl-2-fluorobenzamido)-3-methylbutanoyl)-3-methyl-4-oxo-1,3,8-triazaspiro[4.5]decan-1-yl)phenoxy)acetic acid;
(R)—N-(1-(1-(4-(2-Amino-2-oxoethoxy)phenyl)-3-methyl-4-oxo-1,3,8-triazaspiro[4.5]decan-8-yl)-3-methyl-1-oxobutan-2-yl)-5-ethyl-2-fluorobenzamide;
(R)—N-(1-(1-(4-(Cyanomethoxy)phenyl)-3-methyl-4-oxo-1,3,8-triazaspiro[4.5]decan-8-yl)-3-methyl-1-oxobutan-2-yl)-5-ethyl-2-fluorobenzamide;
(R)—N-(1-(1-(4-(2-(Dimethylamino)ethoxy)phenyl)-3-methyl-4-oxo-1,3,8-triazaspiro[4.5]decan-8-yl)-3-methyl-1-oxobutan-2-yl)-2-fluoro-5-(trifluoromethyl)benzamide trifluoroacetic acid salt;
(R)—N-(1-(1-(4-Acetamidophenyl)-3-methyl-4-oxo-1,3,8-triazaspiro[4.5]decan-8-yl)-3-methyl-1-oxobutan-2-yl)-5-ethyl-2-fluorobenzamide;
(R)-5-Ethyl-2-fluoro-N-(3-methyl-1-(3-methyl-1-(4-(methylsulfonamido)phenyl)-4-oxo-1,3,8-triazaspiro[4.5]decan-8-yl)-1-oxobutan-2-yl)benzamide;
(R)-2-Fluoro-N-(3-methyl-1-(3-methyl-1-(4-(methylsulfonamido)phenyl)-4-oxo-1,3,8-triazaspiro[4.5]decan-8-yl)-1-oxobutan-2-yl)-5-(trifluoromethyl)benzamide;
(R)-Methyl-4-(8-(2-(2-fluoro-5-(trifluoromethyl)benzamido)-3-methylbutanoyl)-3-methyl-4-oxo-1,3,8-triazaspiro[4.5]decan-1-yl)phenylcarbamate;
(R)-2-(8-(2-(3-Fluoro-5-methylbenzamido)-3-methylbutanoyl)-4-oxo-1-phenyl-1,3,8-triazaspiro[4.5]decan-3-yl)acetic acid;
(R)-2-Fluoro-3-methyl-N-(3-methyl-1-(3-(3-(methylsulfonyl)propyl)-4-oxo-1-phenyl-1,3,8-triazaspiro[4.5]decan-8-yl)-1-oxobutan-2-yl)benzamide;
(R)—N-(1-(3-(Cyanomethyl)-4-oxo-1-phenyl-1,3,8-triazaspiro[4.5]decan-8-yl)-3-methyl-1-oxobutan-2-yl)-5-ethyl-2-fluorobenzamide;
(R)—N-(1-(1-(4-Cyanophenyl)-3-methyl-4-oxo-1,3,8-triazaspiro[4.5]decan-8-yl)-3-methyl-1-oxobutan-2-yl)-2-fluoro-3-methylbenzamide;
(R)—N-(1-(1-(3-Cyanophenyl)-3-methyl-4-oxo-1,3,8-triazaspiro[4.5]decan-8-yl)-3-methyl-1-oxobutan-2-yl)-2-fluoro-3-methylbenzamide;
(R)—N-(1-(1-(4-Chlorophenyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(1-(1-(4-fluorophenyl)-3-methyl-2,4-dioxo-1,3,8-triazaspiro[4.5]decan-8-yl)-3-methyl-1-oxobutan-2-yl)-5-(trifluoromethyl)benzamide;
(R)—N-(1-(1-(3,4-Dichlorophenyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(3-methyl-1-(3-methyl-2,4-dioxo-1-p-tolyl-1,3,8-triazaspiro[4.5]decan-8-yl)-1-oxobutan-2-yl)-5-(trifluoromethyl)benzamide;
(R)—N-(1-(1-(1H-Indol-5-yl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(3-methyl-1-(3-methyl-2,4-dioxo-1-(4-(trifluoromethyl)phenyl)-1,3,8-triazaspiro[4.5]decan-8-yl)-1-oxobutan-2-yl)-5-(trifluoromethyl)benzamide;
(R)—N-(1-(1-(3-Chlorophenyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)-2-Fluoro-N-(3-methyl-1-(3-methyl-2,4-dioxo-1-(pyridin-2-yl)-1,3,8-triazaspiro[4.5]decan-8-yl)-1-oxobutan-2-yl)-5-(trifluoromethyl)benzamide;
(R)-2-Fluoro-N-(3-methyl-1-(3-methyl-2,4-dioxo-1-(pyridin-3-yl)-1,3,8-triazaspiro[4.5]decan-8-yl)-1-oxobutan-2-yl)-5-(trifluoromethyl)benzamide;
(R)-2-Fluoro-N-(3-methyl-1-(3-methyl-2,4-dioxo-1-(4-deuterium-phenyl-1,3,8-triazaspiro[4.5]decan-8-yl)-1-oxobutan-2-yl)-5-(trifluoromethyl)benzamide;
(R)—N-(1-(1-(Benzo[d][1,3]dioxol-5-yl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1-(3-Chlorophenyl)-3-methyl-4-oxo-1,3,8-triazaspiro[4.5]decan-8-yl)-3-methyl-1-oxobutan-2-yl)-3-methylbenzamide;
(R)—N-(1-(1-(4-Chlorophenyl)-3-methyl-4-oxo-1,3,8-triazaspiro[4.5]decan-8-yl)-3-methyl-1-oxobutan-2-yl)-3-methylbenzamide;
(R)—N-(1-(1-(3-Bromophenyl)-3-methyl-4-oxo-1,3,8-triazaspiro[4.5]decan-8-yl)-3-methyl-1-oxobutan-2-yl)-3-methylbenzamide;
(R)-3-Methyl-N-(3-methyl-1-(3-methyl-4-oxo-1-(pyridin-3-yl)-1,3,8-triazaspiro[4.5]decan-8-yl)-1-oxobutan-2-yl)benzamide;
(R)—N-(1-(1-(2-Chlorophenyl)-3-methyl-4-oxo-1,3,8-triazaspiro[4.5]decan-8-yl)-3-methyl-1-oxobutan-2-yl)-3-methylbenzamide;
(R)—N-(1-(1-(4-Methoxyphenyl)-3-methyl-4-oxo-1,3,8-triazaspiro[4.5]decan-8-yl)-3-methyl-1-oxobutan-2-yl)-3-methylbenzamide;
(R)—N-(1-(1-(3-Methoxyphenyl)-3-methyl-4-oxo-1,3,8-triazaspiro[4.5]decan-8-yl)-3-methyl-1-oxobutan-2-yl)-3-methylbenzamide;
(R)—N-(1-(1-(4-Bromophenyl)-3-methyl-4-oxo-1,3,8-triazaspiro[4.5]decan-8-yl)-3-methyl-1-oxobutan-2-yl)-3-methylbenzamide;
(R)-5-Ethyl-2-fluoro-N-(3-methyl-1-(3-methyl-1-(4-(methylsulfonyl)phenyl)-4-oxo-1,3,8-triazaspiro[4.5]decan-8-yl)-1-oxobutan-2-yl)benzamide;
(R)-5-Ethyl-2-fluoro-N-(3-methyl-1-(3-methyl-1-(1-methyl-6-oxo-1,6-dihydropyridin-3-yl)-4-oxo-1,3,8-triazaspiro[4.5]decan-8-yl)-1-oxobutan-2-yl)benzamide;
(R)-5-Ethyl-2-fluoro-N-(1-(1-(4-methoxyphenyl)-3-methyl-4-oxo-1,3,8-triazaspiro[4.5]decan-8-yl)-3-methyl-1-oxobutan-2-yl)benzamide;
(R)-5-Ethyl-2-fluoro-N-(3-methyl-1-(3-methyl-1-(3-methyl-2-oxo-2,3-dihydrobenzo[d]oxazol-5-yl)-4-oxo-1,3,8-triazaspiro[4.5]decan-8-yl)-1-oxobutan-2-yl)benzamide;
(R)-2-Fluoro-N-(3-methyl-1-(3-methyl-1-(1-methyl-6-oxo-1,6-dihydropyridin-3-yl)-4-oxo-1,3,8-triazaspiro[4.5]decan-8-yl)-1-oxobutan-2-yl)-5-(trifluoromethyl)benzamide;
(R)-2-Fluoro-N-(3-methyl-1-(3-methyl-1-(4-(methylsulfonyl)phenyl)-4-oxo-1,3,8-triazaspiro[4.5]decan-8-yl)-1-oxobutan-2-yl)-5-(trifluoromethyl)benzamide;
(R)-2-Fluoro-N-(1-(1-(imidazo[1,2-a]pyridin-6-yl)-3-methyl-4-oxo-1,3,8-triazaspiro[4.5]decan-8-yl)-3-methyl-1-oxobutan-2-yl)-5-(trifluoromethyl)benzamide trifluoroacetic acid salt;
(R)-3-Chloro-N-(3-methyl-1-(3-methyl-1-(1-methyl-6-oxo-1,6-dihydropyridin-3-yl)-4-oxo-1,3,8-triazaspiro[4.5]decan-8-yl)-1-oxobutan-2-yl)-5-(trifluoromethyl)benzamide;
(R)—N-(1-Cyclopentyl-2-(3-methyl-1-(1-methyl-6-oxo-1,6-dihydropyridin-3-yl)-4-oxo-1,3,8-triazaspiro[4.5]decan-8-yl)-2-oxoethyl)-2-fluoro-5-(trifluoromethyl)benzamide;
2-Fluoro-N-((2R)-1-(1-(4-methoxylpenyl)-2,3-dimethyl-4-oxo-1,3,8-triazaspiro[4.5]decan-8-yl)-3-methyl-1-oxobutan-2-yl)-5-(trifluoromethyl)benzamide;
4-((R)-8-((R)-2-(5-Ethyl-2-fluorobenzamido)-3-methylbutanoyl)-2,3-dimethyl-4-oxo-1,3,8-triazaspiro[4.5]decan-1-yl)benzoic acid;
4-((S)-8-((R)-2-(5-Ethyl-2-fluorobenzamido)-3-methylbutanoyl)-2,3-dimethyl-4-oxo-1,3,8-triazaspiro[4.5]decan-1-yl)benzoic acid;
4-((R)-8-((R)-2-(2-Fluoro-5-(trifluoromethyl)benzamido)-3-methylbutanoyl)-2,3-dimethyl-4-oxo-1,3,8-triazaspiro[4.5]decan-1-yl)benzoic acid;
4-((S)-8-((R)-2-(2-Fluoro-5-(trifluoromethyl)benzamido)-3-methylbutanoyl)-2,3-dimethyl-4-oxo-1,3,8-triazaspiro[4.5]decan-1-yl)benzoic acid;
(R)—N-(1-(1-(1H-Indazol-5-yl)-3-methyl-4-oxo-1,3,8-triazaspiro[4.5]decan-8-yl)-3-methyl-1-oxobutan-2-yl)-2-fluoro-5-(trifluoromethyl)benzamide;
(R)—N-(1-(1-(1H-Indazol-5-yl)-3-methyl-4-oxo-1,3,8-triazaspiro[4.5]decan-8-yl)-3-methyl-1-oxobutan-2-yl)-5-ethyl-2-fluorobenzamide;
(R)—N-(1-(1-(1H-Benzo[d]imidazol-6-yl)-3-methyl-4-oxo-1,3,8-triazaspiro[4.5]decan-8-yl)-3-methyl-1-oxobutan-2-yl)-5-ethyl-2-fluorobenzamide;
(R)-3-Methyl-N-(3-methyl-1-oxo-1-(2-oxo-1-phenyl-3-oxa-1,8-diazaspiro[4.5]decan-8-yl)butan-2-yl)benzamide;
(R)-2-Fluoro-N-(3-methyl-1-(3-methyl-1-(4-(oxetan-3-ylamino)phenyl)-4-oxo-1,3,8-triazaspiro[4.5]decan-8-yl)-1-oxobutan-2-yl)-5-(trifluoromethyl)benzamide;
(R)-2-Fluoro-N-(1-(1-((3-methoxycyclobutyl)methyl)-3-methyl-2,4-dioxo-1,3,8-triazaspiro[4.5]decan-8-yl)-3-methyl-1-oxobutan-2-yl)-5-(trifluoromethyl)benzamide;
(R)—N-(1-(1-(4-(1H-1,2,4-Triazol-3-yl)phenyl)-3-methyl-4-oxo-1,3,8-triazaspiro[4.5]decan-8-yl)-3-methyl-1-oxobutan-2-yl)-2-fluoro-5-(trifluoromethyl)benzamide;
(R)-Methyl-4-(8-(2-(2-fluoro-5-(trifluoromethyl)benzamido)-3-methylbutanoyl)-3-methyl-2,4-dioxo-1,3,8-triazaspiro[4.5]decan-1-yl)benzoate;
(R)-4-(8-(2-(5-Cyclopropyl-2-fluorobenzamido)-3-methylbutanoyl)-3-methyl-2,4-dioxo-1,3,8-triazaspiro[4.5]decan-1-yl)benzoic acid;
(R)-4-(8-(2-(2-Fluoro-5-(trifluoromethyl)benzamido)-3-methylbutanoyl)-3-methyl-2,4-dioxo-1,3,8-triazaspiro[4.5]decan-1-yl)benzoic acid;
(R)—N-(1-(1-(4-(2H-Tetrazol-5-yl)phenyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1-(4-(2H-Tetrazol-5-yl)phenyl)-3-methyl-2,4-dioxo-1,3,8-triazaspiro[4.5]decan-8-yl)-3-methyl-1-oxobutan-2-yl)-2-fluoro-5-(trifluoromethoxy)benzamide;
(R)—N-(1-(1-(4-(2H-Tetrazol-5-yl)phenyl)-3-methyl-2,4-dioxo-1,3,8-triazaspiro[4.5]decan-8-yl)-3-methyl-1-oxobutan-2-yl)-5-(difluoromethoxy)-2-fluorobenzamide;
(R)—N-(1-(1-(4-(2H-Tetrazol-5-yl)phenyl)-3-methyl-4-oxo-1,3,8-triazaspiro[4.5]decan-8-yl)-3-methyl-1-oxobutan-2-yl)-2-fluoro-5-(trifluoromethyl)benzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)picolinamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)nicotinamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)cyclohexanecarboxamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)isonicotinamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)pivalamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)benzamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)thiazole-2-carboxamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-4-(trifluoromethyl)thiazole-2-carboxamide;
(R)—N-(1-(1-(1H-Indazol-5-yl)-3-methyl-2,4-dioxo-1,3,8-triazaspiro[4.5]decan-8-yl)-3-methyl-1-oxobutan-2-yl)-2-fluoro-N-methyl-5-(trifluoromethyl)benzamide;
(R)-4-(3-(2-(Dimethylamino)ethyl)-8-(2-(2-fluoro-5-(trifluoromethyl)benzamido)-3-methylbutanoyl)-2,4-dioxo-1,3,8-triazaspiro[4.5]decan-1-yl)benzoic acid trifluoroacetic acid salt;
(R)-4-(3-(2-(Dimethylamino)ethyl)-8-(2-(2-fluoro-5-(trifluoromethyl) benzamido)-3-methylbutanoyl)-4-oxo-1,3,8-triazaspiro[4.5]decan-1-yl)benzoic acid trifluoroacetic acid salt;
(R)-2-Fluoro-N-(3-methyl-1-(3-methyl-2,4-dioxo-1-(pyridin-4-yl)-1,3,8-triazaspiro[4.5]decan-8-yl)-1-oxobutan-2-yl)-5-(trifluoromethyl)benzamide;
(R)-4-(8-(2-(2-Fluoro-5-(trifluoromethyl)benzamido)-3-methylbutanoyl)-3-methyl-2,4-dioxo-1,3,8-triazaspiro[4.5]decan-1-yl)pyridine 1-oxide;
(R)-2-Fluoro-N-(1-(1-(4-methoxycyclohexyl)-3-methyl-2,4-dioxo-1,3,8-triazaspiro[4.5]decan-8-yl)-3-methyl-1-oxobutan-2-yl)-5-(trifluoromethyl)benzamide;
(R)—N-(1-(1-Cyclohexyl-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(3-Methyl-1-(3-methyl-2,4-dioxo-1-(4-methoxy-d3-phenyl)-1,3,8-triazaspiro[4.5]decan-8-yl)-1-oxobutan-2-yl)-3-(trifluoromethyl)-5-fluorobenzamide;
(R)—N-(3-Methyl-1-(3-methyl-2,4-dioxo-1-(4-ethoxyphenyl)-1,3,8-triazaspiro[4.5]decan-8-yl)-1-oxobutan-2-yl)-3-(trifluoromethyl)-5-fluorobenzamide;
(R)—N-(1-(1-(Benzo[d]oxazol-5-yl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1-(2-Methylbenzo[d]oxazol-5-yl)-3-methyl-2,4-dioxo-1,3,8-triazaspiro[4.5]decan-8-yl)-3-methyl-1-oxobutan-2-yl)-2-fluoro-5-(trifluoromethyl)benzamide;
or a pharmaceutically acceptable salt thereof.

19. The composition of claim 1, wherein the compound of Formula I is selected from the group consisting of:
(R)-2-Fluoro-N-(3-methyl-1-(3-methyl-2,4-dioxo-1-(2-oxo-2,3-dihydrobenzo[d]oxazol-6-yl)-1,3,8-triazaspiro[4.5]decan-8-yl)-1-oxobutan-2-yl)-5-(trifluoromethyl)benzamide;
(R)-2-Fluoro-N-(3-methyl-2,4-dioxo-1,3,8-triazaspiro[4.5]decan-8-yl)-3-methyl-1-oxobutan-2-yl)-1-(oxobutan-2-yl)-5-(trifluoromethyl)benzamide;
(R)—N-(1-(1-(4-methoxyphenyl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1-(1H-indazol-5-yl)-3-methyl-2,4-dioxo-1,3,8-triazaspiro[4.5]decan-8-yl)-3-methyl-1-oxobutan-2-yl)-2-fluoro-5-(trifluoromethyl)benzamide;
(R)—N-(1-(1-(1H-indazol-5-yl)-3-methyl-2,4-dioxo-1,3,8-triazaspiro[4.5]decan-8-yl)-3-methyl-1-oxobutan-2-yl)-3-(trifluoromethyl)benzamide;
(R)—N-(1-(1-(1H-indazol-5-yl)-3-methyl-2,4-dioxo-1,3,8-triazaspiro[4.5]decan-8-yl)-3-methyl-1-oxobutan-2-yl)-2-fluoro-5-(trifluoromethoxy)benzamide;
(R)-4-(8-(2-(2-Fluoro-5-(trifluoromethyl)benzamido)-3-methylbutanoyl)-3-methyl-4-oxo-1,3,8-triazaspiro[4.5]decan-1-yl)benzoic acid;
(R)—N-(1-(1-(1H-indol-5-yl)-3-methyl-2,4-dioxo-1,3,8-triazaspiro[4.5]decan-8-yl)-3-methyl-1-oxobutan-2-yl)-2-fluoro-5-(trifluoromethyl)benzamide;
4-((R))-8-((R)-2-(5-Ethyl-2-fluorobenzamido)-3-methylbutanoyl)-2,3-dimethyl-4-oxo-1,3,8-triazaspiro[4.5]decan-1-yl)benzoic acid; and
(R)—N-(1-(1-(1H-indazol-5-yl)-3-methyl-2,4-dioxo-1,3,8-triazaspiro[4.5]decan-8-yl)-3-methyl-1-oxobutan-2-yl)nicotinamide,
or a pharmaceutically acceptable salt thereof.

20. The composition of claim 1, wherein the at least one additional therapeutic agent is selected from selected from hedgehog protein inhibitors, smoothened receptor antagonists, endothelin ET-A antagonists, endothelin ET-B antagonists, FGF receptor antagonists, FGF1 receptor antagonists, FGF2 receptor antagonists, PDGF receptor alpha antagonists, PDGF receptor antagonists, PDGF receptor beta antagonists, VEGF receptor antagonists, VEGF-1 receptor antagonists, VEGF-2 receptor antagonists, VEGF-3 receptor antagonists, IL-13 antagonists, interferon beta ligands, mTOR complex 1 inhibitors, TGF beta antagonists, p38 MAP kinase inhibitors, NADPH oxidase 1 inhibitors, NADPH oxidase 4 inhibitors, connective tissue growth factor ligand inhibitors, IL-6 agonists, insulin-like growth factor 1 antagonists, somatostatin receptor agonists, 5-lipoxygenase inhibitors, PDE 3 inhibitors, phospholipase C inhibitors, serum amyloid P stimulator, guanylate cyclase stimulator, PDE 4 inhibitors, Abl tyrosine kinase inhibitors, Kit tyrosine kinase inhibitors, signal transduction inhibitors, angiotensin II ligand modulator, endothelin 1 ligand inhibitors, relaxin agonist, IL-4 antagonist, TNF antagonist, yype II TNF receptor modulator, monocyte chemotactic protein 1 ligand inhibitors, galectin-3 inhibitors, SH2 domain inositol phosphatase 1 stimulator, MAPKAPK2 inhibitors, caspase inhibitors, lysophosphatidate-1 receptor antagonist, beta 2 adrenoceptor agonist, interferon gamma ligands, superoxide dismutase modulator, hyaluronidase stimulator, transaminase stimulator, integrin alpha-V/beta-6 antagonist, a lysyl oxidase-like protein 2 (LOXL2) inhibitor, adrenoceptor antagonist, VIP agonist, interferon alpha ligands, phosphoinositide 3-kinase inhibitors, Jun N terminal kinase inhibitors, collagen V modulators, metalloprotease-9 stimulators, PPAR agonists, adenosine A2b receptor antagonists, GPCR modulators, CCR7 chemokine modulators, interleukin 17E ligand inhibitors, interleukin receptor 17B antagonists, AKT protein kinase inhibitors, hyaluronan mediated motility receptor modulators, angiotensin II AT-2 receptor agonists, CXC11 chemokine ligand modulators, immunoglobulin Fc receptor modulators, lysophosphatidate-1 receptor antagonists, ubiquitin thioesterase inhibitors, 5-HT 2b receptor antagonists, LDL receptor related protein-6 inhibitors, telomerase stimulators, endostatin modulators, Wnt-1 induced signal pathway protein 1 inhibitors, NK1 receptor antagonists, CD95 antagonists, protein tyrosine phosphatase 1E inhibitors, plasminogen activator inhibitors 1 inhibitors, spleen tyrosine kinase inhibitors, MMP9 inhibitors, TPL2 COT Kinase inhibitors, JAK1/2 inhibitors, Bruton's tyrosine kinase (BTK) inhibitors, integrin alpha 4 beta 7 inhibitors, PAD4 inhibitors, PAD2 inhibitors, IRAK4 inhibitors, ASK1 inhibitors, PIM1 inhibitors, PIM3 inhibitors, AMPK inhibitors, IL-17 inhibitors, PD-1 agonist, IL-33 inhibtior, IL-25 inhibitors, and IL-22 agonists.

21. The composition of claim 1, wherein the at least one additional therapeutic agent is selected from the group consisting of a chemotherapeutic agent, an anti-cancer agent, an anti-angiogenic agent, an anti-fibrotic agent, an immunotherapeutic agent, a therapeutic antibody, a radiotherapeutic agent, an anti-neoplastic agent, an anti-proliferation agent, or any combination thereof.

22. The composition of claim 21, wherein the therapeutic agent is selected from an adenosine A2B receptor (A2B) inhibitor, apoptosis signal-regulating kinase (ASK) inhibitor, a BET-bromodomain 4 (BRD4) inhibitor, a Bruton's tyrosine kinase (BTK) inhibitor, a discoidin domain receptor 1 (DDR1) inhibitor, a histone deacetylase (HDAC) inhibitor, an isocitrate dehydrogenase 1 (IDH1) inhibitor, an IKK inhibitor, a Janus kinase (JAK) inhibitor, a lysyl oxidase-like protein 2 (LOXL2) inhibitor, a matrix metalloprotease 9 (MMP9) inhibitor, a phosphatidylinositol 3-kinase (PI3K) inhibitor, a protein kinase C (PKC) activator or inhibitor, a TPL2 inhibitor, a serine/threonine-protein kinase 1 (TBK1) inhibitor, a spleen tyrosine kinase (SYK) inhibitor, agents that activate or reactivate latent human immunodeficiency virus (HIV) such as panobinostat or romidepsin, an anti-CD20 antibody such as obinutuzumab, an anti-programmed cell death protein 1 (anti-PD-1) antibody such as nivolimumab (BMS-936558, MDX1106, or MK-34775), and anti-programmed death-ligand 1 (anti-PD-L1) antibodies such as BMS-936559, MPDL3280A, MEDI4736, MSB0010718C, MDX1105-01.

23. The composition of claim 1, wherein the at least one additional therapeutic agent is selected from the group consisting of anti-anginals, heart failure agents, antithrombotic agents, antiarrhythmic agents, antihypertensive agents, and lipid lowering agents.

24. The composition of claim 23, wherein the anti-anginal is selected from beta-blockers, calcium channel blockers, and nitrates.

25. The composition of claim 23, wherein the heart failure agent is selected from diuretics, ACE inhibitors, vasodilators, and cardiac glycosides.

26. The composition of claim 23, wherein the antithrombotic agent is selected from platelet inhibitors, anticoagulants, and thrombolytic agents.
